(19) United States
(12) Patent Application Publication
(10) Pub. No.: US 2025/0011260 A1
(43) Pub. Date: Jan. 9, 2025

(54) Anthracene Compound For Host Material, Light-Emitting Device, Light-Emitting Apparatus, Electronic Apparatus, and Lighting Apparatus

(71) Applicant: Semiconductor Energy Laboratory Co., Ltd., Kanagawa-ken (JP)

(72) Inventors: Hiroki SUZUKI, Nagoya (JP); Satoshi SEO, Sagamihara (JP); Hiroshi KADOMA, Sagamihara (JP); Tsunenori SUZUKI, Yokohama (JP); Naoaki HASHIMOTO, Sagamihara (JP)

(73) Assignee: Semiconductor Energy Laboratory Co., Ltd., Kanagawa-ken (JP)

(21) Appl. No.: 18/762,410

(22) Filed: Jul. 2, 2024

Related U.S. Application Data
(63) Continuation of application No. 17/256,798, filed on Dec. 29, 2020, now abandoned.

(30) Foreign Application Priority Data
Feb. 14, 2019 (JP) 2019-024416

(51) Int. Cl. C07C 15/28; H10K 50/11; H10K 85/60

(52) U.S. Cl. CPC C07C 15/28; H10K 85/615; C07C 2603/24; H10K 50/11

(57) ABSTRACT

A novel compound for a host material is provided. A compound for a host material that is capable of increasing the lifetime of a light-emitting device is provided. A light-emitting device with a long lifetime is provided. A material whose thermophysical properties such as a glass transition temperature are high is provided. An anthracene compound for a host material represented by General Formula (G1) below is provided. (Note that in General Formula (G1), R1 to R7 each independently represent hydrogen or an aryl group having 1 to 25 carbon atoms.)

This application is a continuation of copending U.S. application Ser. No. 17/256,798, filed on Dec. 29, 2020 which a 371 of international application PCT/IB2020/050890 filed on Feb. 5, 2020 which are all incorporated herein by reference.

### TECHNICAL FIELD

One embodiment of the present invention relates to an anthracene compound for a host material, a light-emitting element, a light-emitting device, a display module, a lighting module, a display device, a light-emitting apparatus, an electronic apparatus, and a lighting apparatus. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting apparatus, a power storage device, a memory device, an imaging device, a driving method thereof, and a manufacturing method thereof.

### BACKGROUND ART

Light-emitting devices (organic EL elements) including organic compounds and utilizing electroluminescence (EL) have been put into practical use. In the basic structure of such light-emitting devices, an organic compound layer containing a light-emitting material (an EL layer) is interposed between a pair of electrodes. Carriers are injected by application of voltage to the element, and recombination energy of the carriers is used, whereby light emission can be obtained from the light-emitting material.

Such light-emitting devices are of self-light-emitting type; thus, they have higher visibility than liquid crystal displays and are suitable as pixels of a display. Displays using such light-emitting devices are also highly advantageous in that they require no backlight and can be fabricated to be thin and lightweight. Moreover, an extremely fast response speed is also a feature.

Since light-emitting layers of such light-emitting devices can be successively formed two-dimensionally, planar light emission can be achieved. This feature is difficult to realize with point light sources typified by incandescent lamps and LEDs or linear light sources typified by fluorescent lamps; thus, the light-emitting devices also have great potential as planar light sources, which can be applied to lighting apparatuses and the like.

Displays or lighting apparatuses using light-emitting devices can be suitably used for a variety of electronic apparatuses as described above, and research and development of light-emitting devices have progressed for higher efficiency and longer lifetime.

The characteristics of light-emitting devices have been improved considerably, but are still insufficient to satisfy advanced requirements for various characteristics such as efficiency and durability. In particular, a decrease in efficiency due to degradation is desirably as small as possible in order to solve the problem such as burn-in, which has been still discussed as a specific problem for EL.

Degradation largely depends on emission center substances or their peripheral materials, which promotes the development of host materials having favorable characteristics.

### REFERENCE

### Patent Document

[Patent Document 1] Japanese Published Patent Application No. 2004-59535

### SUMMARY OF THE INVENTION

### Problems to be Solved by the Invention

An object of one embodiment of the present invention is to provide a novel compound for a host material. Another object of one embodiment of the present invention is to provide a compound for a host material that is capable of increasing the lifetime of a light-emitting device. Another object of one embodiment of the present invention is to provide a light-emitting device with a long lifetime. Another object of one embodiment of the present invention is to provide a material whose thermophysical properties such as a glass transition temperature are high.

An object of another embodiment of the present invention is to provide a light-emitting apparatus, an electronic apparatus, and a display device each having high reliability.

It is only necessary that at least one of the above-described objects be achieved in the present invention.

### Means for Solving the Problems

One embodiment of the present invention is an anthracene compound for a host represented by General Formula (G1) below.

### [Chemical Formula 1]

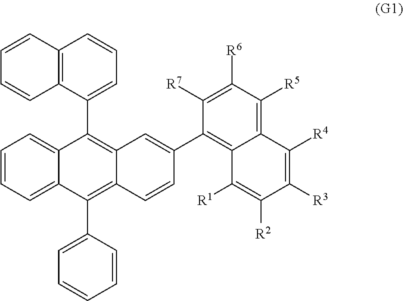

Note that in General Formula (G1), R¹ to R⁷ each independently represent hydrogen or an aryl group having 1 to 25 carbon atoms.

Another embodiment of the present invention is the anthracene compound for a host with the above structure, in which one of R¹ to R⁷ represents an aryl group having 1 to 25 carbon atoms and the others represent hydrogen.

Another embodiment of the present invention is an anthracene compound for a host material represented by General Formula (G2) below.

### [Chemical Formula 2]

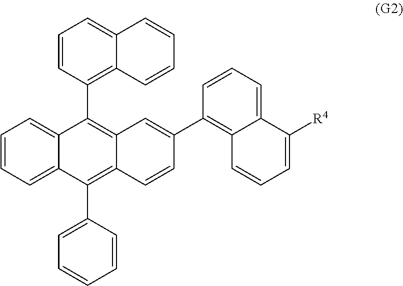

Note that in General Formula (G2), R⁴ represents hydrogen or an aryl group having 1 to 25 carbon atoms.

Another embodiment of the present invention is the anthracene compound for a host material with the above structure, in which the aryl group having 1 to 25 carbon atoms is a phenyl group.

Another embodiment of the present invention is an anthracene compound for a host represented by Structural Formula (100) below.

### [Chemical Formula 3]

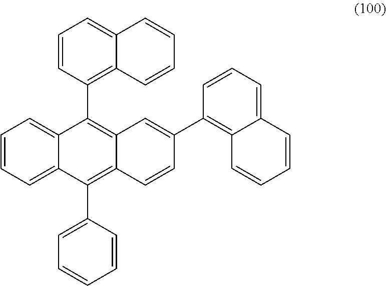 [SMILES: c1ccc(-c2c3ccccc3c(-c3cccc4ccccc34)c3cc(-c4cccc5ccccc45)ccc23)cc1]

Another embodiment of the present invention is a light-emitting device including an anode, a cathode, and an EL layer positioned between the anode and the cathode; the EL layer contains an emission center substance and a host material; and the host material is the anthracene compound for a host material having any of the above structures.

Another embodiment of the present invention is the light-emitting device with the above structure, in which the emission center substance emits blue fluorescence.

Another embodiment of the present invention is a light-emitting apparatus including the light-emitting device having any one of the above structures and a transistor or a substrate.

Another embodiment of the present invention is an electronic apparatus including the light-emitting apparatus having the above structure, and a sensor, an operation button, a speaker, or a microphone.

Another embodiment of the present invention is a lighting apparatus including the light-emitting apparatus having the above structure and a housing.

Note that the light-emitting apparatus in this specification includes, in its category, an image display device that uses a light-emitting device. The light-emitting apparatus may also include a module in which a light-emitting device is provided with a connector such as an anisotropic conductive film or a TCP (Tape Carrier Package), a module in which a printed wiring board is provided at the end of a TCP, and a module in which an IC (integrated circuit) is directly mounted on a light-emitting device by a COG (Chip On Glass) method. Furthermore, in some cases, a lighting device or the like includes the light-emitting apparatus.

### Effect of the Invention

According to one embodiment of the present invention, a novel organic compound can be provided. A novel organic compound with a hole-transport property can be provided. A novel hole-transport material can be provided. A novel light-emitting device can be provided. A light-emitting device with a long lifetime can be provided. A light-emitting device with high emission efficiency can be provided. A light-emitting device with low driving voltage can be provided. A device with a small change in voltage with accumulated driving time can be provided.

According to another embodiment of the present invention, a light-emitting apparatus, an electronic apparatus, and a display device each having high reliability can be provided. According to another embodiment of the present invention, a light-emitting apparatus, an electronic apparatus, and a display device each having low power consumption can be provided.

Note that the description of the effects does not preclude the existence of other effects. Note that one embodiment of the present invention does not need to have all these effects. Note that effects other than these will be apparent from the description of the specification, the drawings, the claims, and the like and effects other than these can be derived from the description of the specification, the drawings, the claims, and the like.

### BRIEF DESCRIPTION OF THE DRAWINGS

FIG. 1A1, FIG. 1A2, FIG. 1B, and FIG. 1C are schematic views of light-emitting devices.

FIG. 2A and FIG. 2B are conceptual views of an active matrix light-emitting apparatus.

FIG. 3A and FIG. 3B are conceptual views of active matrix light-emitting apparatuses.

FIG. 4 is a conceptual view of an active matrix light-emitting apparatus.

FIG. 5A and FIG. 5B are conceptual views of a passive matrix light-emitting apparatus.

FIG. 6A and FIG. 6B are views showing a lighting apparatus.

FIG. 7A, FIG. 7B1, FIG. 7B2, and FIG. 7C are views showing electronic apparatuses.

FIG. 8A, FIG. 8B, and FIG. 8C are views showing electronic apparatuses.

FIG. 9 is a view showing a lighting apparatus.

FIG. 10 is a view showing a lighting apparatus.

FIG. 11 is a view showing in-vehicle display devices and lighting apparatuses.

FIG. 12A and FIG. 12B are views showing an electronic apparatus.

FIG. 13A, FIG. 13B, and FIG. 13C are views showing an electronic apparatus.

FIG. 14A and FIG. 14B are ¹H-NMR charts of 2αN-αNPhA.

FIG. 15 shows the absorption spectrum and the emission spectrum of 2αN-αNPhA in a toluene solution.

FIG. 16 shows the absorption spectrum and the emission spectrum of a thin film of 2αN-αNPhA.

FIG. 17A and FIG. 17B are ¹H-NMR charts of 2PαN-αNPhA.

FIG. 18 shows the absorption spectrum and the emission spectrum of 2PαN-αNPhA in a toluene solution.

FIG. 19 shows the absorption spectrum and the emission spectrum of a thin film of 2PαN-αNPhA.

FIG. 20 shows the luminance-current density characteristics of a light-emitting device 1, a comparative light-emitting device 1, and a comparative light-emitting device 2.

FIG. 21 shows the current efficiency-luminance characteristics of the light-emitting device 1, the comparative light-emitting device 1, and the comparative light-emitting device 2.

FIG. 22 shows the luminance-voltage characteristics of the light-emitting device 1, the comparative light-emitting device 1, and the comparative light-emitting device 2.

FIG. 23 shows the current-voltage characteristics of the light-emitting device 1, the comparative light-emitting device 1, and the comparative light-emitting device 2.

FIG. 24 shows the external quantum efficiency-luminance characteristics of the light-emitting device 1, the comparative light-emitting device 1, and the comparative light-emitting device 2.

FIG. 25 shows the emission spectra of the light-emitting device 1, the comparative light-emitting device 1, and the comparative light-emitting device 2.

FIG. 26 shows the normalized luminance-time change characteristics of the light-emitting device 1, the comparative light-emitting device 1, and the comparative light-emitting device 2.

FIG. 27 shows the luminance-current density characteristics of a light-emitting device 2, a comparative light-emitting device 3, and a comparative light-emitting device 4.

FIG. 28 shows the current efficiency-luminance characteristics of the light-emitting device 2, the comparative light-emitting device 3, and the comparative light-emitting device 4.

FIG. 29 shows the luminance-voltage characteristics of the light-emitting device 2, the comparative light-emitting device 3, and the comparative light-emitting device 4.

FIG. 30 shows the current-voltage characteristics of the light-emitting device 2, the comparative light-emitting device 3, and the comparative light-emitting device 4.

FIG. 31 shows the external quantum efficiency-luminance characteristics of the light-emitting device 2, the comparative light-emitting device 3, and the comparative light-emitting device 4.

FIG. 32 shows the emission spectra of the light-emitting device 2, the comparative light-emitting device 3, and the comparative light-emitting device 4.

FIG. 33 shows the normalized luminance-time change characteristics of the light-emitting device 2, the comparative light-emitting device 3, and the comparative light-emitting device 4.

FIG. 34 shows the luminance-current density characteristics of a light-emitting device 3, a light-emitting device 4, and a comparative light-emitting device 5 to a comparative light-emitting device 10.

FIG. 35 shows the current efficiency-luminance characteristics of the light-emitting device 3, the light-emitting device 4, and the comparative light-emitting device 5 to the comparative light-emitting device 10.

FIG. 36 shows the luminance-voltage characteristics of the light-emitting device 3, the light-emitting device 4, and the comparative light-emitting device 5 to the comparative light-emitting device 10.

FIG. 37 shows the current-voltage characteristics of the light-emitting device 3, the light-emitting device 4, and the comparative light-emitting device 5 to the comparative light-emitting device 10.

FIG. 38 shows the external quantum efficiency-luminance characteristics of the light-emitting device 3, the light-emitting device 4, and the comparative light-emitting device 5 to the comparative light-emitting device 10.

FIG. 39 shows the emission spectra of the light-emitting device 3, the light-emitting device 4, and the comparative light-emitting device 5 to the comparative light-emitting device 10.

FIG. 40 shows the luminance-current density characteristics of a light-emitting device 5, a comparative light-emitting device 11, and a comparative light-emitting device 12.

FIG. 41 shows the current efficiency-luminance characteristics of the light-emitting device 5, the comparative light-emitting device 11, and the comparative light-emitting device 12.

FIG. 42 shows the luminance-voltage characteristics of the light-emitting device 5, the comparative light-emitting device 11, and the comparative light-emitting device 12.

FIG. 43 shows the current-voltage characteristics of the light-emitting device 5, the comparative light-emitting device 11, and the comparative light-emitting device 12.

FIG. 44 shows the external quantum efficiency-luminance characteristics of the light-emitting device 5, the comparative light-emitting device 11, and the comparative light-emitting device 12.

FIG. 45 shows the emission spectra of the light-emitting device 5, the comparative light-emitting device 11, and the comparative light-emitting device 12.

FIG. 46 shows the normalized luminance-time change characteristics of the light-emitting device 5, the comparative light-emitting device 11, and the comparative light-emitting device 12.

### MODE FOR CARRYING OUT THE INVENTION

Embodiments of the present invention are described in detail below with reference to drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

### Embodiment 1

An anthracene compound for a host material of one embodiment of the present invention is an organic compound represented by General Formula (G1) below.

### [Chemical Formula 4]

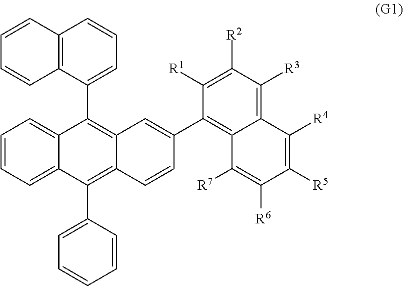

Note that in General Formula (G1), R¹ to R⁷ each independently represent hydrogen or an aryl group having 6 to 25 carbon atoms.

Examples of the aryl group having 6 to 25 carbon atoms include an anthryl group, a phenanthryl group, a pyrenyl group, a triphenylenyl group, a fluoranthenyl group, a biphenyl group, a terphenyl group, and a quaterphenyl group.

It is preferable that R¹ to R⁷ be all hydrogen, or one of R¹ to R⁷ be an aryl group having 6 to 25 carbon atoms and the others be hydrogen. In the case where one of R¹ to R⁷ is an aryl group having 6 to 25 carbon atoms and the others are hydrogen, it is more preferable that R⁴ be an aryl group as represented by General Formula (G2) below.

### [Chemical Formula 5]

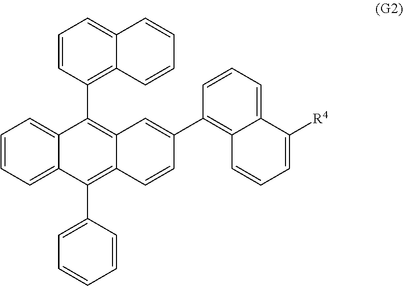

When the anthracene compound for a host material of one embodiment of the present invention, which has the above structure, is used as a host material in a light-emitting layer of a light-emitting device using an organic compound, a light-emitting device with a long lifetime can be provided.

Note that the light-emitting device using as a host material the above compound represented by General Formula (G1) can have a longer lifetime than a light-emitting device using as a host material a compound in which a substituent is bonded to any of a naphthyl group and a phenyl group bonded to the 9-position and the 10-position of an anthracene skeleton in the above compound represented by General Formula (G1).

Similarly, the light-emitting device using as a host material the above compound represented by General Formula (G1) can have a longer lifetime than a light-emitting device using as a host material a compound in which an alkyl group or an alkylsilyl group is bonded to any of naphthyl groups bonded to the 9-position and the 2-position of the anthracene skeleton in the above compound represented by General Formula (G1). Note that a light-emitting device using as a host material a compound in which an aryl group having 6 to 25 carbon atoms is bonded to a naphthyl group bonded to the 2-position of the anthracene skeleton in the above compound represented by General Formula (G1) can have a long lifetime.

Specific examples of the organic compound having the above structure are shown below.

### [Chemical Formula 6]

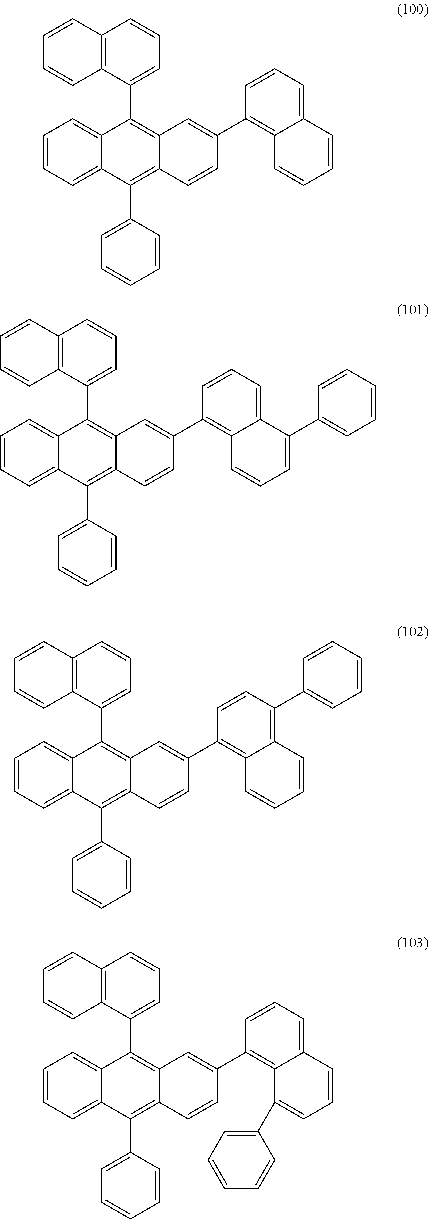

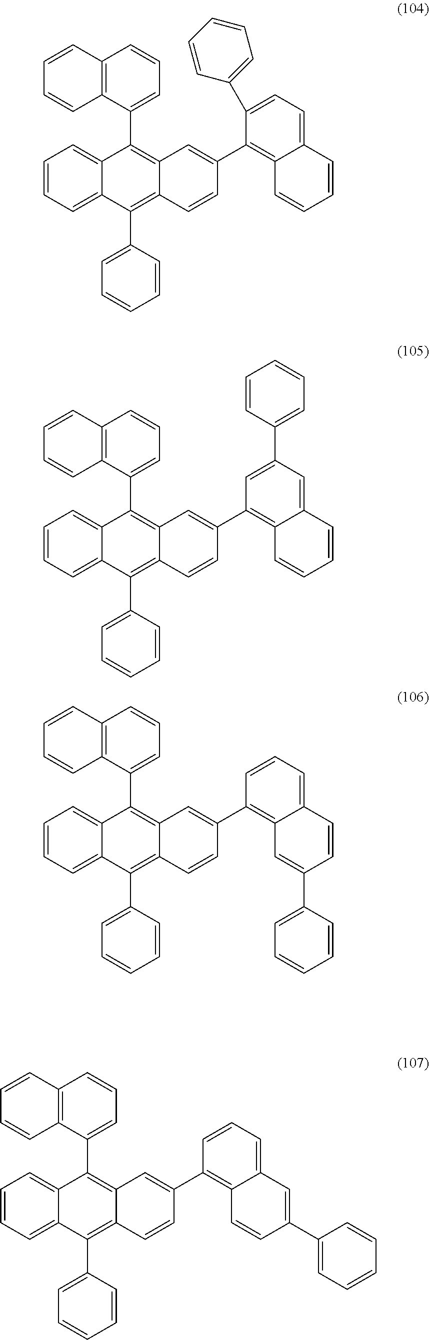

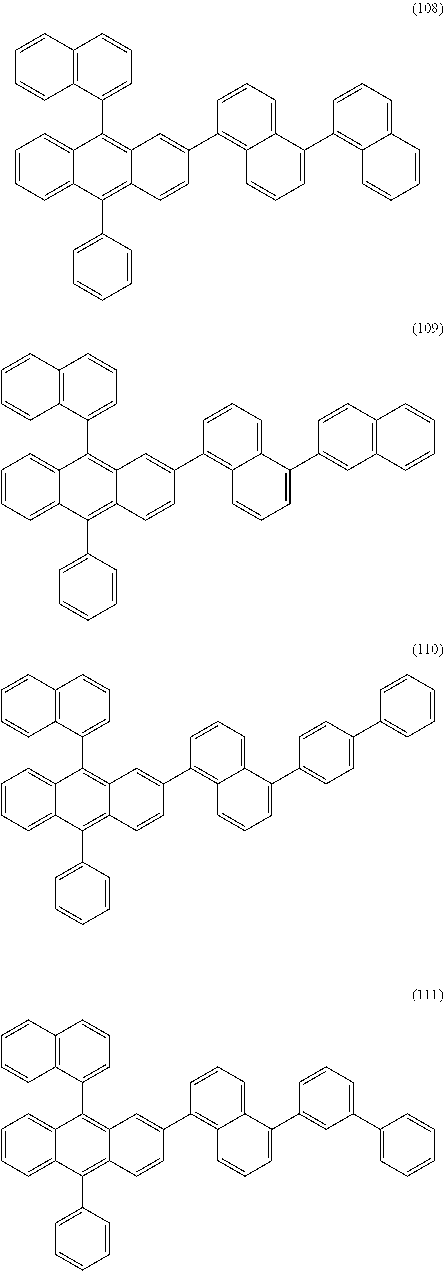

### [Chemical Formula 7]

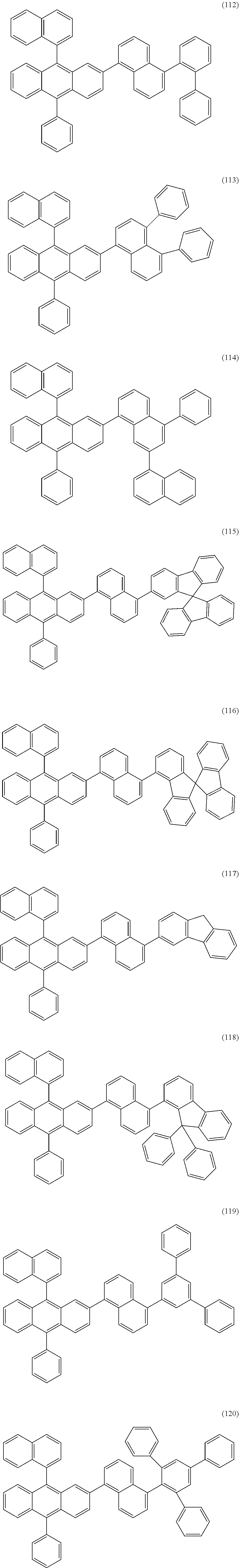

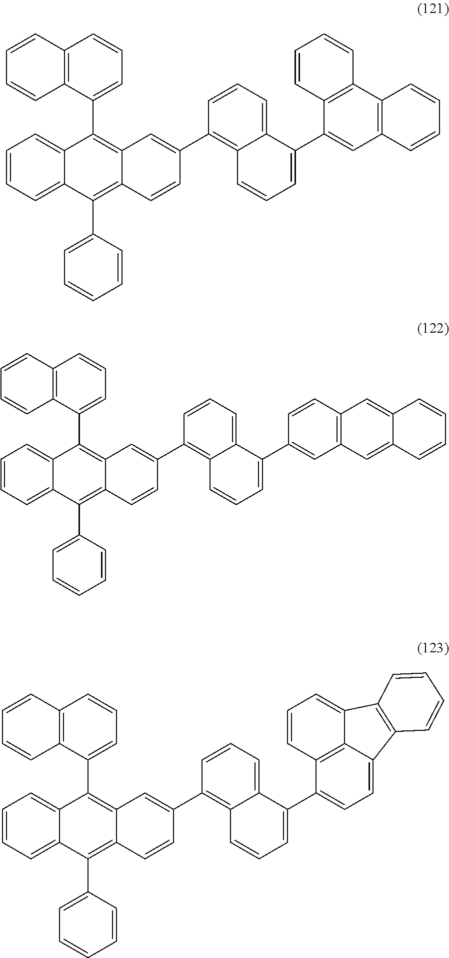

The organic compounds shown above can be synthesized by the following synthesis scheme, for example.

The anthracene compound for a host material (G1) of one embodiment of the present invention can be synthesized by the following synthesis scheme. That is, a halogen compound of an anthracene derivative or a compound of an anthracene derivative that has a triflate group (a1) is coupled with boronic acid or an organoboron compound of a naphthalene compound (a2) by the Suzuki-Miyaura reaction; thus, the anthracene compound (G1) of one embodiment of the present invention can be obtained.

### [Chemical Formula 8]

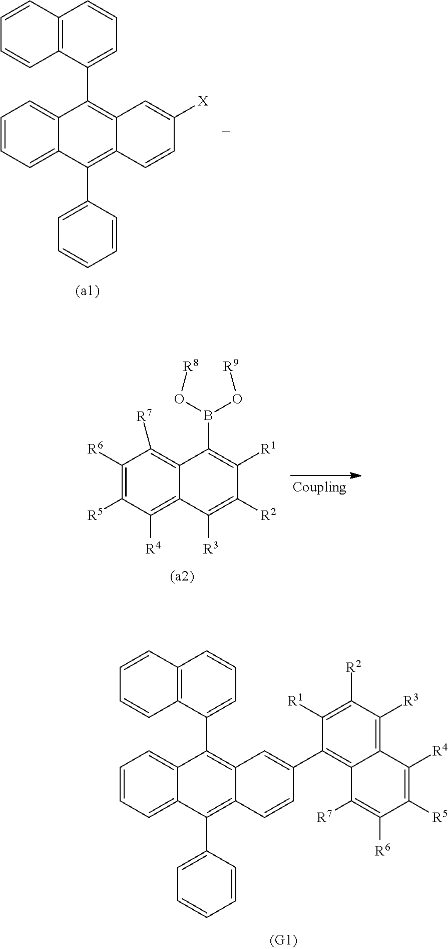

In the above synthesis scheme, R¹ to R⁷ each independently represent any one of hydrogen and an aryl group having 6 to 25 carbon atoms. R⁸ and R⁹ each independently represent any of hydrogen and an alkyl group having 1 to 6 carbon atoms, and R⁸ and R⁹ may be bonded with each other to form a ring.

In addition, X represents a halogen or a triflate group and is particularly preferably chlorine, bromine, or iodine when X is a halogen.

Examples of a palladium catalyst that can be used in the reaction represented by the above synthesis scheme include palladium(II) acetate, tetrakis(triphenylphosphine)palladium(0), and bis(triphenylphosphine)palladium(II) dichloride.

Examples of a ligand in the above palladium catalyst include di(1-adamantyl)-n-butylphosphine, tri(ortho-tolyl)phosphine, triphenylphosphine, and tricyclohexylphosphine.

Examples of the base that can be used in the reaction represented by the above synthesis scheme include an organic base such as sodium tert-butoxide and an inorganic base such as potassium carbonate or sodium carbonate.

Examples of a solvent that can be used in the reaction represented by the above synthesis scheme include a mixed solvent of toluene and water; a mixed solvent of toluene, alcohol such as ethanol, and water; a mixed solvent of xylene and water; a mixed solvent of xylene, alcohol such as ethanol, and water; a mixed solvent of benzene and water; a mixed solvent of benzene, alcohol such as ethanol, and water; a mixed solvent of water and an ether such as ethylene glycol dimethyl ether; and a mixed solvent of an ether such as ethylene glycol dimethyl ether and alcohol such as ethanol. Note that the solvent that can be used is not limited to these. It is more preferable to use a mixed solvent of toluene and water or toluene, ethanol, and water; a mixed solvent of water and an ether such as ethylene glycol dimethyl ether; or a mixed solvent of an ether such as ethylene glycol dimethyl ether and alcohol such as ethanol.

As the coupling reaction that can used in the above synthesis scheme, the Suzuki-Miyaura coupling reaction using the organoboron compound or the boronic acid represented by the compound (a2) may be replaced with a cross coupling reaction using an organoaluminum compound, an organozirconium compound, an organozinc compound, an organotin compound, or the like. In the reaction shown in the above synthesis scheme, an organoboron compound or a boronic acid of an anthracene compound may be coupled with a halide of a naphthalene compound or a naphthalene compound having triflate as a substituent by the Suzuki-Miyaura reaction.

The anthracene compound for a host material of one embodiment of the present invention can be synthesized in the aforementioned manner.

### Embodiment 2

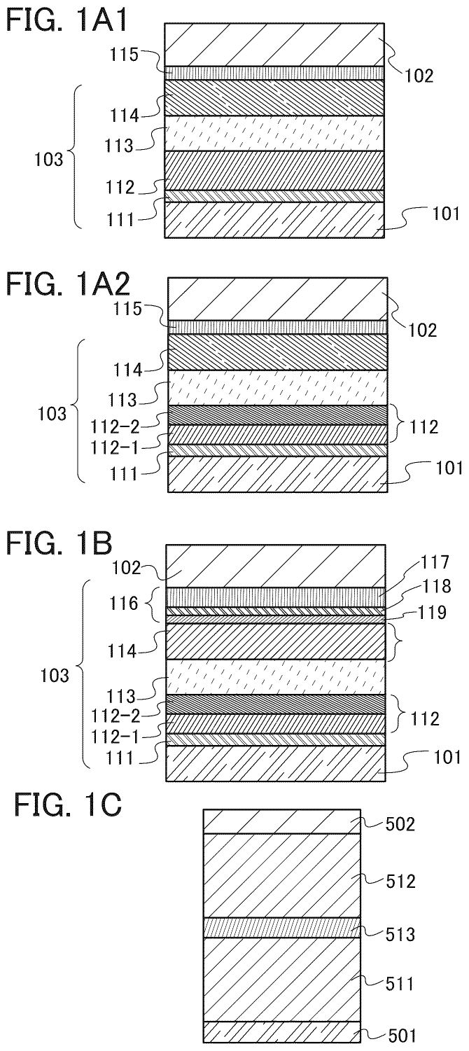

FIG. 1A1 shows a diagram illustrating a light-emitting device of one embodiment of the present invention. The light-emitting device of one embodiment of the present invention includes a first electrode 101, a second electrode 102, and an EL layer 103; the EL layer 103 includes a light-emitting layer 113; and the light-emitting layer contains the anthracene derivative for a host material of one embodiment of the present invention described in Embodiment 1.

The EL layer 103 may include a hole-injection layer 111, a hole-transport layer 112, an electron-transport layer 114, and an electron-injection layer 115 in addition to the light-emitting layer 113, and may also include other various layers such as a carrier-blocking layer, an exciton-blocking layer, and a charge-generation layer. Note that as shown in FIG. 1A2, the hole-transport layer 112 may include two layers of a first hole-transport layer 112-1 and a second hole-transport layer 112-2, which are formed of different materials. Note that the second hole-transport layer 112-2 also functions as an electron-blocking layer.

The anthracene compound for a host material is used as a host material contained in the light-emitting layer 113. The light-emitting device of one embodiment of the present invention, which uses the anthracene compound for a host material as a host material, can have a long lifetime.

The first electrode 101 is preferably formed using any of metals, alloys, and conductive compounds with a high work function (specifically, higher than or equal to 4.0 eV), mixtures thereof, and the like. Specific examples include indium oxide-tin oxide (ITO: Indium Tin Oxide), indium oxide-tin oxide containing silicon or silicon oxide, indium oxide-zinc oxide, and indium oxide containing tungsten oxide and zinc oxide (IWZO). Such conductive metal oxide films are usually formed by a sputtering method, but may be formed by application of a sol-gel method or the like. In an example of the formation method, indium oxide-zinc oxide is deposited by a sputtering method using a target obtained by adding 1 to 20 wt % of zinc oxide to indium oxide. Furthermore, indium oxide containing tungsten oxide and zinc oxide (IWZO) can be deposited by a sputtering method using a target in which tungsten oxide and zinc oxide are added to indium oxide at 0.5 to 5 wt % and 0.1 to 1 wt %, respectively. Alternatively, gold (Au), platinum (Pt), nickel (Ni), tungsten (W), chromium (Cr), molybdenum (Mo), iron (Fe), cobalt (Co), copper (Cu), palladium (Pd), nitride of a metal material (e.g., titanium nitride), or the like can be used. Graphene can also be used. Note that when a composite material described later is used for a layer that is in contact with the first electrode 101 in the EL layer 103, an electrode material can be selected regardless of its work function.

Two types of stacked-layer structure of the EL layer 103 are described in this embodiment: the structure shown in FIG. 1A1, which includes the electron-transport layer 114 and the electron-injection layer 115 in addition to the hole-injection layer 111, the hole-transport layer 112, and the light-emitting layer 113; and the structure shown in FIG. 1B, which includes the electron-transport layer 114 and a charge-generation layer 116 in addition to the hole-injection layer 111, the hole-transport layer 112, and the light-emitting layer 113. Materials for forming each layer will be specifically described below.

The hole-injection layer 111 contains a substance having an acceptor property. As the substance having an acceptor property, a compound having an electron-withdrawing group (a halogen group or a cyano group) can be used; for example, compounds having an electron-withdrawing group such as 7,7,8,8-tetracyano-2,3,5,6-tetrafluoroquinodimethane (abbreviation: F4-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), and 1,3,4,5,7,8-hexafluorotetracyano-naphthoquinodimethane (abbreviation: F6-TCNNQ) can be used. As an organic compound having an acceptor property, a compound in which electron-withdrawing groups are bonded to a condensed aromatic ring having a plurality of heteroatoms, such as HAT-CN, is preferable because it is thermally stable. A [3]radialene derivative having an electron-withdrawing group (in particular, a cyano group or a halogen group such as a fluoro group) is preferable because of having a very high electron-accepting property. Specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α,”-1,2,3-cyclopropanetriylidenetris[2,3,4,5,6-pentafluorobenzeneacetonitrile]. As the substance having an acceptor property, molybdenum oxide, vanadium oxide, ruthenium oxide, tungsten oxide, manganese oxide, or the like can be used, other than the above-described organic compounds. Alternatively, the hole-injection layer 111 can be formed using a phthalocyanine-based complex compound such as phthalocyanine (abbreviation: H₂Pc) and copper phthalocyanine (CuPc), an aromatic amine compound such as 4,4′-bis[N-(4-diphenylaminophenyl)-N-phenylamino]biphenyl (abbreviation: DPAB) and N,N-bis{4-[bis(3-methylphenyl)amino]phenyl}-N,N′-diphenyl-(1,1′-biphenyl)-4,4′-diamine (abbreviation: DNTPD), or a high molecular compound such as poly(3,4-ethylenedioxythiophene)/poly(styrenesulfonic acid) (PEDOT/PSS). The substance having an acceptor property can extract electrons from an adjacent hole-transport layer (or hole-transport material) by the application of an electric field.

Alternatively, a composite material in which a substance having a hole-transport property contains an acceptor substance can be used for the hole-injection layer 111. By using a composite material in which a substance having a hole-transport property contains an acceptor substance, a material used to form an electrode can be selected regardless of its work function. In other words, besides a material having a high work function, a material having a low work function can also be used for the first electrode 101. As the acceptor substance, any of the aforementioned substances having an acceptor property can be used; among them, molybdenum oxide is preferable because it is stable in the air, has a low hygroscopic property, and is easy to handle.

As the substance with a hole-transport property used for the composite material, any of a variety of organic compounds such as aromatic amine compounds, carbazole derivatives, aromatic hydrocarbons, and high molecular compounds (e.g., oligomers, dendrimers, or polymers) can be used. Note that the substance with a hole-transport property used for the composite material preferably has a hole mobility of 10⁻⁶ cm²/Vs or higher. Note that the organic compound of one embodiment of the present invention can also be suitably used. Organic compounds which can be used as the substance with a hole-transport property in the composite material are specifically given below.

Examples of the aromatic amine compounds that can be used for the composite material include N,N-di(p-tolyl)-N,-diphenyl-p-phenylenediamine (abbreviation: DTDPPA), 4,4′-bis[N-(4-diphenylaminophenyl)-N-phenylamino]biphenyl (abbreviation: DPAB), N,N-bis{4-[bis(3-methylphenyl)amino]phenyl}-N,N′-diphenyl-(1,1′-biphenyl)-4,4′-diamine (abbreviation: DNTPD), and 1,3,5-tris[N-(4-diphenylaminophenyl)-N-phenylamino]benzene (abbreviation: DPA3B). Specific examples of the carbazole derivative include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon may have a vinyl skeleton. Examples of the aromatic hydrocarbon having a vinyl group include 4,4′-bis(2,2-diphenylvinyl)biphenyl (abbreviation: DPVBi) and 9,10-bis[4-(2,2-diphenylvinyl)phenyl]anthracene (abbreviation: DPVPA).

Other examples include high molecular compounds such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide] (abbreviation: PTPDMA), and poly[N,N-bis(4-butylphenyl)-N,N-bis(phenyl)benzidine] (abbreviation: Poly-TPD).

The formation of the hole-injection layer 111 can improve the hole-injection property, which allows the light-emitting device to be driven at a low voltage. In addition, the organic compound having an acceptor property is easy to use because it is easily deposited by vapor deposition.

The hole-transport layer 112 contains a hole-transport material. The hole-transport material preferably has a hole mobility of 1×10⁻⁶ cm²/Vs or higher. As the hole-transport material, any of the aforementioned organic compounds used as the hole-transport materials in the composite material can be used.

The light-emitting layer 113 is a layer containing a light-emitting material and a host material. The light-emitting material may be fluorescent substances, phosphorescent substances, substances exhibiting thermally activated delayed fluorescence (TADF), or other light-emitting materials. Furthermore, it may be a single layer or be formed of a plurality of layers including different light-emitting materials. Note that one embodiment of the present invention is more suitably used in the case where the light-emitting layer 113 is a layer that exhibits fluorescence, specifically, a layer that exhibits blue fluorescence.

Examples of a material that can be used as a fluorescent substance in the light-emitting layer 113 are as follows. Fluorescent substances other than those given below can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-diphenyl-N,N-bis[4-(9-phenyl-9H-fluoren-9-yl)phenyl]pyrene-1,6-diamine (abbreviation: 1,6FLPAPrn), N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-(tert-butyl)perylene (abbreviation: TBP), 4-(10-phenyl-9-anthryl)-4′-(9-phenyl-9H-carbazol-3-yl)triphenylamine (abbreviation: PCBAPA), N,N′-(2-tert-butylanthracene-9,10-diyldi-4,1-phenylene)bis[N,N,N-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-(pyrene-1,6-diyl)bis[(6,N-diphenylbenzo[b]naphtho[1,2-d]furan)-8-amine](abbreviation:1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Condensed aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, and high reliability.

In the case where a phosphorescent substance is used as an emission center material in the light-emitting layer 113, the following materials can be used, for example.

The examples are as follows: an organometallic iridium complex having a 4H-triazole skeleton, such as tris{2-[5-(2-methylphenyl)-4-(2,6-dimethylphenyl)-4H-1,2,4-triazol-3-yl-KN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), and tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[1-(2,6-diisopropylphenyl)-2-phenyl-1H-imidazole]iridium(III) (abbreviation: [Ir(iPrpmi)₃]) and tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C²′}iridium(III) picolinate (abbreviation: [Ir(CF₃ppy)₂(pic)]), and bis[2-(4′,6′-difluorophenyl)pyridinato-N,C²′]iridium(III) acetylacetonate (abbreviation: FIr(acac)). These compounds emit blue phosphorescence and have an emission peak at 440 nm to 520 nm.

Other examples include organometallic iridium complexes having a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes having a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), and bis(2-phenylquinolinato-N,C^{2′})iridium(III) acetylacetonate (abbreviation: [Ir(pq)₂(acac)]); and a rare earth metal complex such as tris(acetylacetonato) (monophenanthroline)terbium(III) (abbreviation: [Tb(acac)₃(Phen)]). These are mainly compounds that emit green phosphorescence and have an emission peak at 500 nm to 600 nm. Note that organometallic iridium complexes having a pyrimidine skeleton have distinctively high reliability and emission efficiency and thus are particularly preferable.

Other examples include organometallic iridium complexes having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato(dipivaloylmethanato)iridium(III) (abbreviation: [Ir(d1npm)₂(dpm)]); organometallic iridium complexes having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm])]), and (acetylacetonato)bis[2,3-bis(4-fluorophenyl)quinoxalinato]iridium(III) (abbreviation: [Ir(Fdpq)₂(acac)]); organometallic iridium complexes having a pyridine skeleton, such as tris(1-phenylisoquinolinato-N,C^{2′})iridium(III) (abbreviation: [Ir(piq)₃]) and bis(1-phenylisoquinolinato-N,C²′)iridium(III) acetylacetonate (abbreviation: [Ir(piq)₂(acac)]); platinum complexes such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato) (monophenanthroline)europium(III) (abbreviation: [Eu(DBM)₃(Phen)]) and tris[l-(2-thenoyl)-3,3,3-trifluoroacetonato(monophenanthroline)europium(III) (abbreviation: [Eu(TTA)₃(Phen)]). These compounds emit red phosphorescence and have an emission peak at 600 nm to 700 nm. Furthermore, the organometallic iridium complexes having a pyrazine skeleton can provide red light emission with favorable chromaticity.

Besides the above-described phosphorescent compounds, other known phosphorescent materials may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

### [Chemical Formula 9]

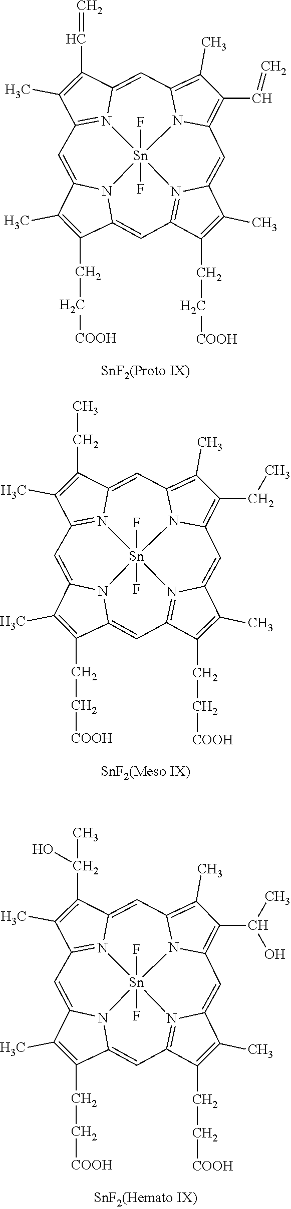

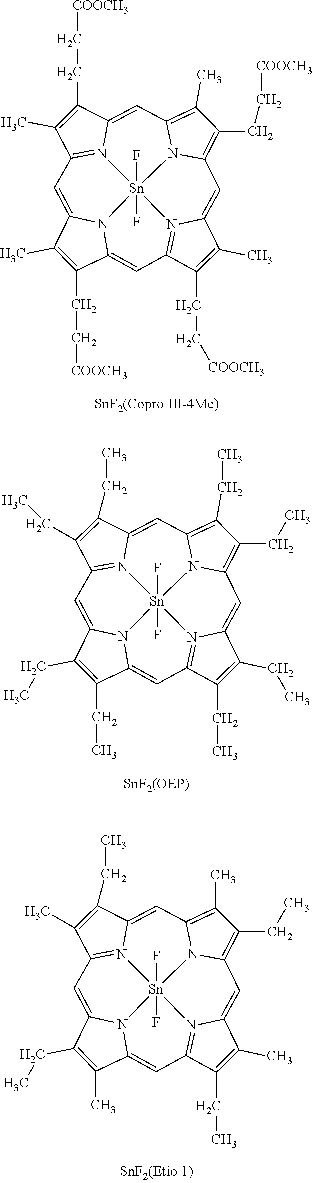

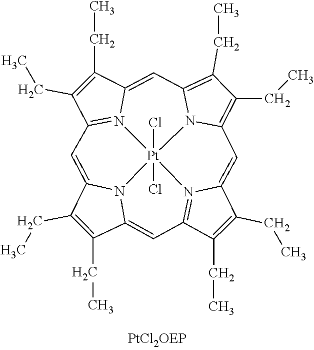

Alternatively, a heterocyclic compound having one of both of a π-electron rich heteroaromatic ring and a π-electron deficient heteroaromatic ring that is represented by the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 2-{4-[3-(N-phenyl-9H-carbazol-3-yl)-9H-carbazol-9-yl]phenyl}-4,6-diphenyl-1,3,5-triazine (abbreviation: PCCzPTzn), 2-[4-(10H-phenoxazine-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA) can be used. The heterocyclic compound is preferable because of having both a high electron-transport property and a high hole-transport property owing to a π-electron rich heteroaromatic ring and a π-electron deficient heteroaromatic ring. Among skeletons having a π-electron deficient heteroaromatic ring, a pyridine skeleton, a diazine skeleton (a pyrimidine skeleton, a pyrazine skeleton, or a pyridazine skeleton) and a triazine skeleton are particularly preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor property and reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; therefore, at least one of these skeletons is preferably included. Note that a dibenzofuran skeleton and a dibenzothiophene skeleton are preferable as the furan skeleton and the thiophene skeleton, respectively. As the pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring and a π-electron deficient heteroaromatic ring are directly bonded to each other is particularly preferable because the electron-donating property of the π-electron rich heteroaromatic ring and the electron-accepting property of the π-electron deficient heteroaromatic ring are both increased and the energy difference between the Si level and the Ti level becomes small, so that thermally activated delayed fluorescence can be obtained efficiently. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a boron-containing skeleton such as phenylborane or boranthrene, an aromatic ring or a heteroaromatic ring having a nitrile group or a cyano group, such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

### [Chemical Formula 10]

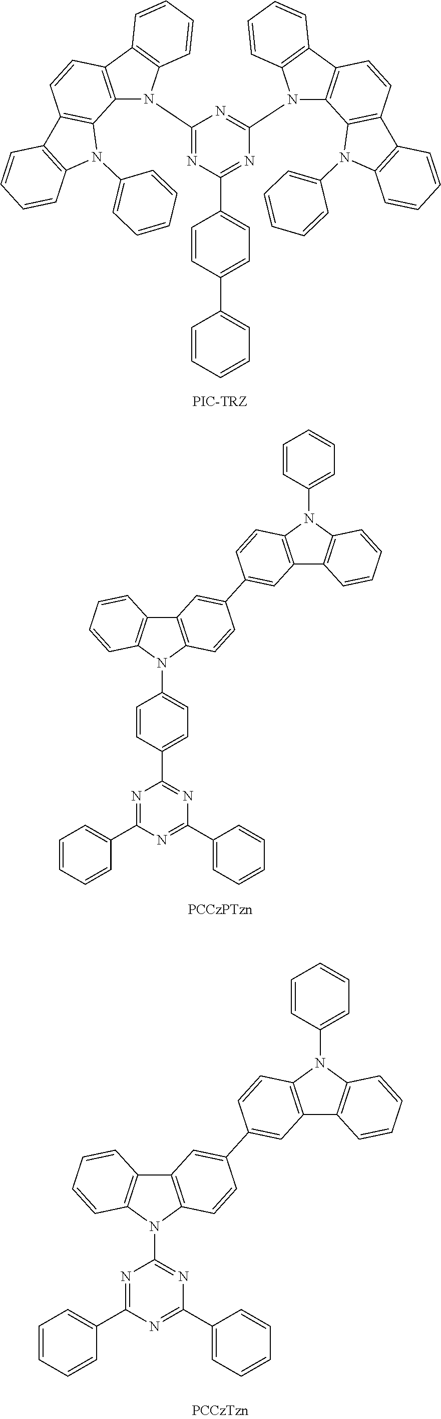 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1]

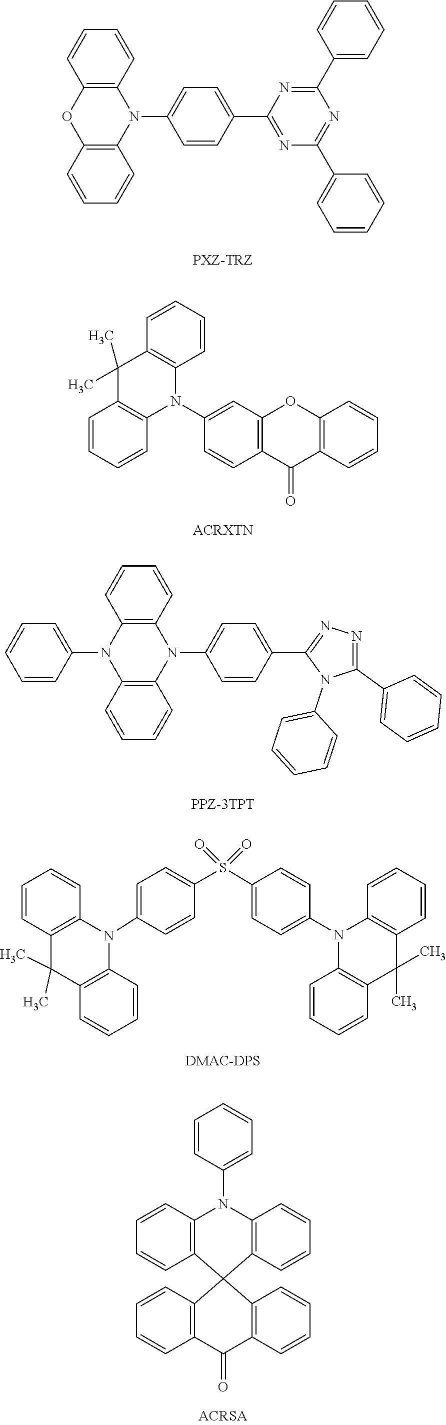

Note that the TADF material is a material that has a small difference between the S1 level and the T1 level and has a function of converting triplet excitation energy into singlet excitation energy by reverse intersystem crossing. Thus, it is possible to upconvert triplet excitation energy into singlet excitation energy (reverse intersystem crossing) using a little thermal energy and to efficiently generate a singlet excited state. In addition, the triplet excitation energy can be converted into luminescence.

An exciplex (also referred to as Exciplex) whose excited state is formed by two kinds of substances has an extremely small difference between the S1 level and the T1 level and functions as a TADF material that can convert triplet excitation energy into singlet excitation energy.

Note that a phosphorescent spectrum observed at low temperatures (e.g., 77 K to 10 K) is used for an index of the T1 level. When the level of energy with a wavelength of the line obtained by extrapolating a tangent to the fluorescent spectrum at a tail on the short wavelength side is the Si level and the level of energy with a wavelength of the line obtained by extrapolating a tangent to the phosphorescent spectrum at a tail on the short wavelength side is the T 1 level, the difference between S1 and T1 of the TADF material is preferably smaller than or equal to 0.3 eV, further preferably smaller than or equal to 0.2 eV.

When the TADF material is used as an emission center material, the S1 level of the host material is preferably higher than that of the TADF material. In addition, the Ti level of the host material is preferably higher than that of the TADF material.

The anthracene compound for a host material of one embodiment of the present invention described in Embodiment 1 is preferably used as the host material in the light-emitting layer. The use of the anthracene compound for a host material can provide a light-emitting device with a long lifetime.

In the case where the anthracene compound for a host material described in Embodiment 1 is not used as the host material, a variety of carrier-transport materials such as a material having an electron-transport property or a material having a hole-transport property can be used.

Examples of the material having a hole-transport property include compounds having an aromatic amine skeleton such as 4,4′-bis[N-(1-naphthyl)-N-phenylamino]biphenyl (abbreviation: NPB), N,N-bis(3-methylphenyl)-N,N-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), and N-phenyl-N-[4-(9-phenyl-9H-carbazol-3-yl)phenyl]spiro-9,9′-bifluoren-2-amine (abbreviation: PCBASF); compounds having a carbazole skeleton such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), and 3,3′-bis(9-phenyl-9H-carbazole) (abbreviation: PCCP); compounds having a thiophene skeleton such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV); and compounds having a furan skeleton such as 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage. The organic compounds described in Embodiment 1 can also be suitably used.

As the material having an electron-transport property, for example, a metal complex such as bis(10-hydroxybenzo[h]quinolinato)beryllium(II) (abbreviation: BeBq₂), bis(2-methyl-8-quinolinolato) (4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ), a heterocyclic compound having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 2-[3′-(9,9-dimethyl-9H-fluoren-2-yl)-1,1′-biphenyl-3-yl]-4,6-diphenyl-1,3,5-triazine (abbreviation: mFBPTzn), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 2-{4-[9,10-di(naphthalen-2-yl)-2-anthryl]phenyl}-1-phenyl-1H-benzimidazole (abbreviation: ZADN), a heterocyclic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), or 4,6-bis[3-(4-dibenzothienyl)phenyl]pyrimidine (abbreviation: 4,6mDBTP2Pm-II), and a heterocyclic compound having a pyridine skeleton, such as 3,5-bis[3-(9H-carbazol-9-yl)phenyl]pyridine (abbreviation: 35DCzPPy) or 1,3,5-tri[3-(3-pyridyl)-phenyl]benzene (abbreviation: TmPyPB) can be given. Among the above, the heterocyclic compound having a diazine skeleton and the heterocyclic compound having a pyridine skeleton are preferable because of having high reliability. In particular, the heterocyclic compound having a diazine (pyrimidine or pyrazine) skeleton has a high electron-transport property and contributes to a reduction in driving voltage.

In the case where a fluorescent substance is used as the light-emitting material, a material having an anthracene skeleton is favorably used as the host material. The use of a substance having an anthracene skeleton as the host material for the fluorescent substance makes it possible to obtain a light-emitting layer with high emission efficiency and high durability. Most of materials having an anthracene skeleton have a deep HOMO level; therefore, one embodiment of the present invention can be suitably used. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton because the hole-injection and hole-transport properties are improved; further preferably, the host material has a benzocarbazole skeleton in which a benzene ring is further condensed to carbazole because the HOMO level thereof is shallower than that of carbazole by approximately 0.1 eV and thus holes enter the host material easily. In particular, the host material preferably has a dibenzocarbazole skeleton because the HOMO level thereof is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzo fluorene skeleton may be used. Examples of such a substance include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), and 9-phenyl-10-{4-(9-phenyl-9H-fluoren-9-yl)biphenyl-4′-yl}anthracene (abbreviation: FLPPA). In particular, CzPA, cgDBCzPA, 2mBnfPPA, and PCzPA are preferably selected because they exhibit excellent characteristics.

Note that the light-emitting device of one embodiment of the present invention is particularly preferably applied to a light-emitting device that emits blue fluorescence.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The ratio of the content of the material having a hole-transport property to the content of the material having an electron-transport property may be 1:9 to 9:1.

An exciplex may be formed of these mixed materials. These mixed materials are preferably selected so as to form an exciplex that exhibits light emission whose wavelength overlaps with the wavelength on a lowest-energy-side absorption band of the light-emitting material, in which case energy can be transferred smoothly and light emission can be obtained efficiently. The use of such a structure is preferable because the driving voltage can also be reduced.

The electron-transport layer 114 contains a substance having an electron-transport property. As the substance having an electron-transport property, it is possible to use any of the above-listed substances having electron-transport properties that can be used as the host material.

A layer containing an alkali metal, an alkaline earth metal, or a compound thereof such as lithium fluoride (LiF), 8-hydroxyquinolinato-lithium (abbreviation: Liq), cesium fluoride (CsF), or calcium fluoride (CaF₂) may be provided as the electron-injection layer 115 between the electron-transport layer 114 and the second electrode 102. An electride or a layer that is formed using a substance having an electron-transport property and that includes an alkali metal, an alkaline earth metal, or a compound thereof can be used as the electron-injection layer 115. Examples of the electride include a substance in which electrons are added at high concentration to calcium oxide-aluminum oxide.

Instead of the electron-injection layer 115, the charge-generation layer 116 may be provided (FIG. 1). The charge-generation layer 116 refers to a layer capable of injecting holes into a layer in contact with the cathode side of the charge-generation layer 116 and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film containing the above-described acceptor material as a material included in the composite material and a film containing a hole-transport material. When a potential is applied to the p-type layer 117, electrons are injected into the electron-transport layer 114 and holes are injected into the second electrode 102 serving as a cathode; thus, the light-emitting device operates.

Note that the charge-generation layer 116 preferably includes one or both of an electron-relay layer 118 and an electron-injection buffer layer 119 in addition to the p-type layer 117.

The electron-relay layer 118 contains at least a substance having an electron-transport property and has a function of preventing an interaction between the electron-injection buffer layer 119 and the p-type layer 117 and smoothly transferring electrons. The LUMO level of the substance having an electron-transport property contained in the electron-relay layer 118 is preferably between the LUMO level of the acceptor substance in the p-type layer 117 and the LUMO level of a substance contained in a layer of the electron-transport layer 114 that is in contact with the charge-generation layer 116. As a specific value of the energy level, the LUMO level of the substance having an electron-transport property in the electron-relay layer 118 is preferably higher than or equal to −5.0 eV, more preferably higher than or equal to −5.0 eV and lower than or equal to −3.0 eV. Note that as the substance having an electron-transport property in the electron-relay layer 118, a phthalocyanine-based material or a metal complex having a metal-oxygen bond and an aromatic ligand is preferably used.

A substance having a high electron-injection property, such as an alkali metal, an alkaline earth metal, a rare earth metal, or a compound thereof (an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)), can be used for the electron-injection buffer layer 119.

In the case where the electron-injection buffer layer 119 contains the substance having an electron-transport property and a donor substance, an organic compound such as tetrathianaphthacene (abbreviation: TTN), nickelocene, or decamethylnickelocene can be used as the donor substance, as well as an alkali metal, an alkaline earth metal, a rare earth metal, a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)). As the substance having an electron-transport property, a material similar to the above-described material for the electron-transport layer 114 can be used.

As a substance for forming the second electrode 102, a metal, an alloy, an electrically conductive compound, or a mixture thereof each having a low work function (specifically, lower than or equal to 3.8 eV) or the like can be used. Specific examples of such a cathode material are elements belonging to Group 1 or Group 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the second electrode 102 and the electron-transport layer, for the second electrode 102, a variety of conductive materials such as Al, Ag, ITO, or indium oxide-tin oxide containing silicon or silicon oxide can be used regardless of the work function. Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Furthermore, any of a variety of methods can be used for forming the EL layer 103, regardless of a dry method or a wet method. For example, a vacuum evaporation method, a gravure printing method, an offset printing method, a screen printing method, an ink-jet method, a spin coating method, or the like may be used.

Different methods may be used to form each of the electrodes or layers described above.

The structure of the layers provided between the first electrode 101 and the second electrode 102 is not limited to the above-described structure. Preferably, a light-emitting region where holes and electrons recombine is positioned away from the first electrode 101 and the second electrode 102 so as to prevent quenching due to the proximity of the light-emitting region and a metal used for electrodes and carrier-injection layers.

Furthermore, in order that transfer of energy from an exciton generated in the light-emitting layer can be suppressed, preferably, the hole-transport layer and the electron-transport layer which are in contact with the light-emitting layer 113, particularly a carrier-transport layer closer to the recombination region in the light-emitting layer 113, are formed using a substance having a wider band gap than the light-emitting material of the light-emitting layer or the light-emitting material included in the light-emitting layer.

Next, an embodiment of a light-emitting device with a structure where a plurality of light-emitting units are stacked (also referred to as a stacked-type device or a tandem device) will be described with reference to FIG. 1C. This light-emitting device is a light-emitting device including a plurality of light-emitting units between an anode and a cathode. One light-emitting unit has substantially the same structure as that of the EL layer 103, which is shown in FIG. 1A1, FIG. 1A2, FIG. 1B, and the like. In other words, the light-emitting device shown in FIG. 1C is a light-emitting device including a plurality of light-emitting units, and the light-emitting device shown in FIG. 1A1, FIG. 1A2, and FIG. 1B is a light-emitting device including one light-emitting unit.

In FIG. 1C, a first light-emitting unit 511 and a second light-emitting unit 512 are stacked between an anode 501 and a cathode 502, and a charge-generation layer 513 is provided between the first light-emitting unit 511 and the second light-emitting unit 512. The anode 501 and the cathode 502 correspond, respectively, to the first electrode 101 and the second electrode 102 in FIG. 1A1 and the like, and the materials given in the description for FIG. 1A1 can be used. Furthermore, the first light-emitting unit 511 and the second light-emitting unit 512 may have the same structure or different structures.

The charge-generation layer 513 has a function of injecting electrons into one of the light-emitting units and injecting holes into the other of the light-emitting units when a voltage is applied between the anode 501 and the cathode 502. That is, in FIG. 1C, the charge-generation layer 513 injects electrons into the first light-emitting unit 511 and holes into the second light-emitting unit 512 when a voltage is applied so that the potential of the anode becomes higher than the potential of the cathode.

The charge-generation layer 513 preferably has a structure similar to that of the charge-generation layer 116 described with reference to FIG. 1B. A composite material of an organic compound and a metal oxide has an excellent carrier-injection property and an excellent carrier-transport property; thus, low-voltage driving and low-current driving can be achieved. In the case where the anode-side surface of a light-emitting unit is in contact with the charge-generation layer 513, the charge-generation layer 513 can also function as a hole-injection layer of the light-emitting unit; therefore, a hole-injection layer is not necessarily provided in the light-emitting unit.

In the case where the charge-generation layer 513 includes the electron-injection buffer layer 119, the electron-injection buffer layer 119 functions as the electron-injection layer in the light-emitting unit on the anode side and thus, an electron-injection layer is not necessarily formed in the light-emitting unit on the anode side.

The light-emitting device having two light-emitting units is described with reference to FIG. 1C; however, one embodiment of the present invention can also be applied to a light-emitting device in which three or more light-emitting units are stacked. With a plurality of light-emitting units partitioned by the charge-generation layer 513 between a pair of electrodes as in the light-emitting device of this embodiment, it is possible to provide a long-life device which can emit light with high luminance at a low current density. A light-emitting apparatus which can be driven at a low voltage and has low power consumption can be achieved.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as the whole. In the case of a three-layer structure, the emission color of the first light-emitting unit may be blue, the emission colors of the second light-emitting unit may be red and green, and the emission color of the third light-emitting unit may be blue so that the light-emitting device can emit white light.

The above-described layers and electrodes such as the EL layer 103, the first light-emitting unit 511, the second light-emitting unit 512, and the charge-generation layer can be formed by a method such as an evaporation method (including a vacuum evaporation method), a droplet discharge method (also referred to as an ink-jet method), a coating method, or a gravure printing method. A low molecular material, a middle molecular material (including an oligomer and a dendrimer), or a high molecular material may be included in the layers and electrodes.

### Embodiment 3

In this embodiment, a light-emitting apparatus using the light-emitting device described in Embodiment 1 and Embodiment 2 will be described.

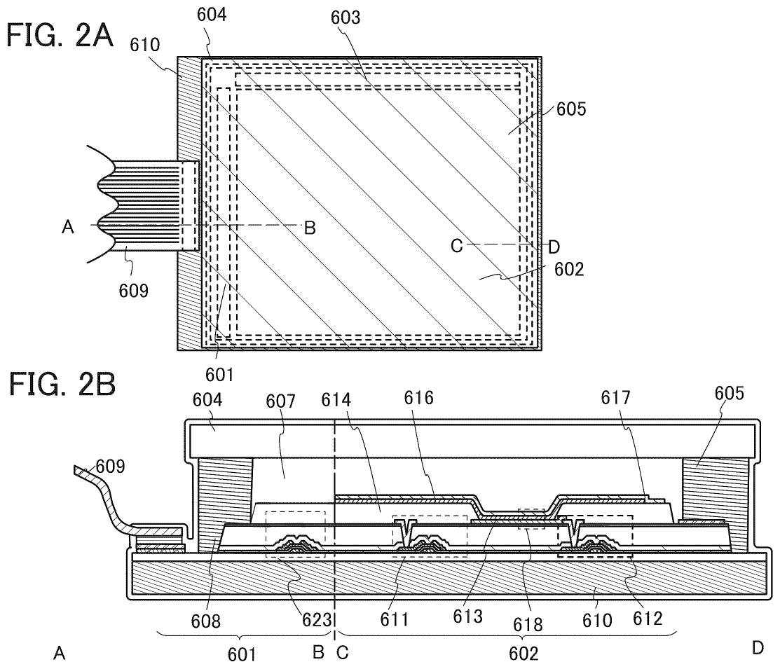

In this embodiment, a light-emitting apparatus fabricated using the light-emitting device described in Embodiment 1 and Embodiment 2 will be described with reference to FIG. 2. Note that FIG. 2A is a top view showing the light-emitting apparatus, and FIG. 2B is a cross-sectional view taken along the lines A-B and C-D in FIG. 2A. This light-emitting apparatus includes a driver circuit portion (source line driver circuit) 601, a pixel portion 602, and a driver circuit portion (gate line driver circuit) 603, which are for controlling light emission of a light-emitting device and are illustrated with dotted lines. Furthermore, 604 denotes a sealing substrate, 605 denotes a sealant, and the inside surrounded by the sealant 605 is a space 607.

Note that a lead wiring 608 is a wiring for transmitting signals to be input to the source line driver circuit 601 and the gate line driver circuit 603 and receiving a video signal, a clock signal, a start signal, a reset signal, and the like from an FPC (flexible printed circuit) 609 serving as an external input terminal. Although only the FPC is illustrated here, a printed wiring board (PWB) may be attached to this FPC. The light-emitting apparatus in this specification includes not only the light-emitting apparatus itself but also the apparatus provided with the FPC or the PWB.

Next, a cross-sectional structure will be described with reference to FIG. 2B. The driver circuit portion and the pixel portion are formed over an element substrate 610; here, the source line driver circuit 601, which is the driver circuit portion, and one pixel in the pixel portion 602 are shown.

The element substrate 610 may be fabricated using a substrate containing glass, quartz, an organic resin, a metal, an alloy, a semiconductor, or the like, or a plastic substrate formed of FRP (Fiber Reinforced Plastics), PVF (polyvinyl fluoride), polyester, acrylic, or the like.

There is no particular limitation on the structure of transistors used in pixels and driver circuits. For example, an inverted staggered transistor or a staggered transistor may be used. Furthermore, top-gate transistors or bottom-gate transistors may be used. There is no particular limitation on a semiconductor material used for the transistors, and for example, silicon, germanium, silicon carbide, gallium nitride, or the like can be used. Alternatively, an oxide semiconductor containing at least one of indium, gallium, and zinc, such as In—Ga—Zn-based metal oxide, may be used.

There is no particular limitation on the crystallinity of a semiconductor material used for the transistors, and any of an amorphous semiconductor and a semiconductor having crystallinity (a microcrystalline semiconductor, a polycrystalline semiconductor, a single crystal semiconductor, or a semiconductor partly including crystal regions) may be used. A semiconductor having crystallinity is preferably used, in which case deterioration of the transistor characteristics can be suppressed.

Here, an oxide semiconductor is preferably used for semiconductor devices such as the transistors provided in the pixels and driver circuits and transistors used for touch sensors described later, and the like. In particular, an oxide semiconductor having a wider band gap than silicon is preferably used. The use of an oxide semiconductor having a wider band gap than silicon can reduce the off-state current of the transistors.

The oxide semiconductor preferably contains at least indium (In) or zinc (Zn). Further preferably, the oxide semiconductor contains an oxide represented by an In-M-Zn-based oxide (M is a metal such as Al, Ti, Ga, Ge, Y, Zr, Sn, La, Ce, or Hf).

An oxide semiconductor that can be used in one embodiment of the present invention is described below.

Oxide semiconductors can be classified into a single crystal oxide semiconductor and a non-single-crystal oxide semiconductor. Examples of the non-single-crystal oxide semiconductor include a CAAC-OS (c-axis aligned crystalline oxide semiconductor), a polycrystalline oxide semiconductor, an nc-OS (nano crystalline oxide semiconductor), an amorphous-like oxide semiconductor (a-like OS), and an amorphous oxide semiconductor.

The CAAC-OS has c-axis alignment, a plurality of nanocrystals are connected in the a-b plane direction, and its crystal structure has distortion. Note that the distortion refers to a portion where the direction of a lattice arrangement changes between a region with a regular lattice arrangement and another region with a regular lattice arrangement in a region where the plurality of nanocrystals are connected.

The nanocrystal is basically a hexagon but is not always a regular hexagon and is a non-regular hexagon in some cases. Furthermore, a pentagonal or heptagonal lattice arrangement, for example, is included in the distortion in some cases. Note that it is difficult to observe a clear crystal grain boundary (also referred to as grain boundary) even in the vicinity of distortion in the CAAC-OS. That is, formation of a crystal grain boundary is found to be inhibited by the distortion of a lattice arrangement. This is because the CAAC-OS can tolerate distortion owing to a low density of arrangement of oxygen atoms in the a-b plane direction, an interatomic bond length changed by substitution of a metal element, and the like.

The CAAC-OS tends to have a layered crystal structure (also referred to as a layered structure) in which a layer containing indium and oxygen (hereinafter, an In layer) and a layer containing the element M, zinc, and oxygen (hereinafter, an (M,Zn) layer) are stacked. Note that indium and the element M can be replaced with each other, and when the element Min the (M,Zn) layer is replaced with indium, the layer can also be referred to as an (In,M,Zn) layer. Furthermore, when indium in the In layer is replaced with the element M, the layer can be referred to as an (In,M) layer.

The CAAC-OS is an oxide semiconductor with high crystallinity. On the other hand, a clear crystal grain boundary cannot be observed in the CAAC-OS; thus, it can be said that a reduction in electron mobility due to the crystal grain boundary is less likely to occur. Entry of impurities, formation of defects, or the like might decrease the crystallinity of an oxide semiconductor; thus, it can be said that the CAAC-OS is an oxide semiconductor that has small amounts of impurities and defects (e.g., oxygen vacancies (also referred to as VO)). Thus, an oxide semiconductor including the CAAC-OS is physically stable. Therefore, the oxide semiconductor including the CAAC-OS is resistant to heat and has high reliability.

In the nc-OS, a microscopic region (e.g., a region with a size greater than or equal to 1 nm and less than or equal to 10 nm, in particular, a region with a size greater than or equal to 1 nm and less than or equal to 3 nm) has a periodic atomic arrangement. Furthermore, there is no regularity of crystal orientation between different nanocrystals in the nc-OS. Thus, the orientation in the whole film is not observed. Accordingly, the nc-OS cannot be distinguished from an a-like OS or an amorphous oxide semiconductor by some analysis methods.

Note that indium-gallium-zinc oxide (hereinafter referred to as IGZO) that is a kind of oxide semiconductor containing indium, gallium, and zinc has a stable structure in some cases by being formed of the above-described nanocrystals. In particular, crystals of IGZO tend not to grow in the air and thus, a stable structure is obtained when IGZO is formed of smaller crystals (e.g., the above-described nanocrystals) rather than larger crystals (here, crystals with a size of several millimeters or several centimeters).

The a-like OS is an oxide semiconductor having a structure between those of the nc-OS and the amorphous oxide semiconductor. The a-like OS includes a void or a low-density region. That is, the a-like OS has low crystallinity compared with the nc-OS and the CAAC-OS.

An oxide semiconductor has various structures with different properties. Two or more of the amorphous oxide semiconductor, the polycrystalline oxide semiconductor, the a-like OS, the nc-OS, and the CAAC-OS may be included in an oxide semiconductor of one embodiment of the present invention.

As an oxide semiconductor other than the above, a CAC (Cloud-Aligned Composite)-OS may be used.

A CAC-OS has a conducting function in part of the material and has an insulating function in another part of the material; as a whole, the CAC-OS has a function of a semiconductor. Note that in the case where the CAC-OS is used in a semiconductor layer of a transistor, the conducting function is a function that allows electrons (or holes) serving as carriers to flow, and the insulating function is a function that does not allow electrons serving as carriers to flow. By the complementary action of the conducting function and the insulating function, a switching function (On/Off function) can be given to the CAC-OS. In the CAC-OS, separation of the functions can maximize each function.

In addition, the CAC-OS includes conductive regions and insulating regions. The conductive regions have the above-described conducting function, and the insulating regions have the above-described insulating function. Furthermore, in some cases, the conductive regions and the insulating regions in the material are separated at the nanoparticle level. Furthermore, in some cases, the conductive regions and the insulating regions are unevenly distributed in the material. Furthermore, in some cases, the conductive regions are observed to be coupled in a cloud-like manner with their boundaries blurred.

In the CAC-OS, the conductive regions and the insulating regions each have a size greater than or equal to 0.5 nm and less than or equal to 10 nm, preferably greater than or equal to 0.5 nm and less than or equal to 3 nm, and are dispersed in the material, in some cases.

The CAC-OS is composed of components having different band gaps. For example, the CAC-OS is composed of a component having a wide gap due to the insulating region and a component having a narrow gap due to the conductive region. In the case of the structure, when carriers flow, carriers mainly flow in the component having a narrow gap. Furthermore, the component having a narrow gap complements the component having a wide gap, and carriers also flow in the component having a wide gap in conjunction with the component having a narrow gap. Therefore, in the case where the above-described CAC-OS is used in a channel formation region of a transistor, the transistor in the on state can achieve high current driving capability, that is, high on-state current and high field-effect mobility.

In other words, the CAC-OS can also be referred to as a matrix composite or a metal matrix composite.

The use of the aforementioned oxide semiconductor material for the semiconductor layer makes it possible to achieve a highly reliable transistor in which a change in the electrical characteristics is reduced.

Charge accumulated in a capacitor through a transistor including the above-described semiconductor layer can be retained for a long time because of the low off-state current of the transistor. The use of such a transistor in pixels allows a driver circuit to stop while the gray level of an image displayed on each display region is maintained. As a result, an electronic apparatus with significantly reduced power consumption can be achieved.

For stable characteristics of the transistor, a base film is preferably provided. The base film can be formed to be a single layer or a stacked layer using an inorganic insulating film such as a silicon oxide film, a silicon nitride film, a silicon oxynitride film, or a silicon nitride oxide film. The base film can be formed by a sputtering method, a CVD (Chemical Vapor Deposition) method (e.g., a plasma CVD method, a thermal CVD method, or an MOCVD (Metal Organic CVD) method), an ALD (Atomic Layer Deposition) method, a coating method, a printing method, or the like. Note that the base film is not necessarily provided when not needed.

Note that an FET 623 is illustrated as a transistor formed in the driver circuit portion 601. The driver circuit can be formed using various circuits such as a CMOS circuit, a PMOS circuit, and an NMOS circuit. Although a driver-integrated type in which the driver circuit is formed over the substrate is described in this embodiment, the driver circuit is not necessarily formed over the substrate and can be formed outside.

The pixel portion 602 is formed with a plurality of pixels including a switching FET 611, a current control FET 612, and a first electrode 613 electrically connected to a drain of the current control FET 612; however, without being limited thereto, a pixel portion in which three or more FETs and a capacitor are combined may be employed.

Note that an insulator 614 is formed to cover an end portion of the first electrode 613. The insulator 614 can be formed using a positive photosensitive acrylic here.

In order to improve the coverage with an EL layer or the like to be formed later, the insulator 614 is formed so as to have a curved surface with curvature at its upper end portion or lower end portion. For example, in the case where positive photosensitive acrylic is used as a material for the insulator 614, only the upper end portion of the insulator 614 preferably has a curved surface with a curvature radius (0.2 μm to 3 μm). For the insulator 614, either a negative photosensitive resin or a positive photosensitive resin can be used.

An EL layer 616 and a second electrode 617 are formed over the first electrode 613. Here, as a material used for the first electrode 613, a material with a high work function is desirably used. For example, a single-layer film of an ITO film, an indium tin oxide film containing silicon, an indium oxide film containing zinc oxide at 2 to 20 wt %, a titanium nitride film, a chromium film, a tungsten film, a Zn film, a Pt film, or the like, a stacked layer of a titanium nitride film and a film containing aluminum as its main component, a three-layer structure of a titanium nitride film, a film containing aluminum as its main component, and a titanium nitride film, or the like can be used. Note that the stacked-layer structure achieves low wiring resistance, a favorable ohmic contact, and a function as an anode.

The EL layer 616 is formed by any of a variety of methods such as an evaporation method using an evaporation mask, an inkjet method, and a spin coating method. The EL layer 616 has the structure described in Embodiment 1 and Embodiment 2. Alternatively, a material included in the EL layer 616 may be a low molecular compound or a high molecular compound (including an oligomer or a dendrimer).

As a material used for the second electrode 617, which is formed over the EL layer 616, a material with a low work function (e.g., Al, Mg, Li, Ca, or an alloy or a compound thereof (MgAg, MgIn, AlLi, or the like)) is preferably used. Note that in the case where light generated in the EL layer 616 passes through the second electrode 617, it is preferable to use, for the second electrode 617, a stacked layer of a thin metal film and a transparent conductive film (e.g., ITO, indium oxide containing zinc oxide at 2 to 20 wt %, indium tin oxide containing silicon, or zinc oxide (ZnO)).

Note that a light-emitting device is formed with the first electrode 613, the EL layer 616, and the second electrode 617. The light-emitting device is the light-emitting device described in Embodiment 1 and Embodiment 2. A plurality of light-emitting devices are formed in the pixel portion, and the light-emitting apparatus of this embodiment may include both the light-emitting device described in Embodiment 1 and Embodiment 2 and a light-emitting device having a different structure.

The sealing substrate 604 and the element substrate 610 are attached to each other using the sealant 605, achieving a structure in which a light-emitting device 618 is provided in the space 607 surrounded by the element substrate 610, the sealing substrate 604, and the sealant 605. The space 607 is filled with a filler; it is filled with an inert gas (e.g., nitrogen or argon) in some cases, and filled with the sealant in some cases. The sealing substrate in which a recessed portion is formed and a desiccant is provided therein is preferable because deterioration due to the influence of moisture can be inhibited.

Note that an epoxy-based resin or glass frit is preferably used for the sealant 605. Furthermore, these materials are preferably materials that transmit moisture or oxygen as little as possible. For the sealing substrate 604, in addition to a glass substrate and a quartz substrate, a plastic substrate formed of FRP (Fiber Reinforced Plastics), PVF (polyvinyl fluoride), polyester, acrylic, or the like can be used.

Although not shown in FIG. 2, a protective film may be provided over the cathode. As the protective film, an organic resin film or an inorganic insulating film can be formed. The protective film may be formed so as to cover an exposed portion of the sealant 605. The protective film may be provided so as to cover surfaces and side surfaces of the pair of substrates and exposed side surfaces of a sealing layer, an insulating layer, and the like.

For the protective film, a material that is less likely to transmit an impurity such as water can be used. Thus, diffusion of an impurity such as water from the outside into the inside can be effectively inhibited.

As a material included in the protective film, an oxide, a nitride, a fluoride, a sulfide, a ternary compound, a metal, a polymer, or the like can be used; for example, it is possible to use a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, or indium oxide; or a material containing aluminum nitride, hafnium nitride, silicon nitride, tantalum nitride, titanium nitride, niobium nitride, molybdenum nitride, zirconium nitride, or gallium nitride; a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like.

The protective film is preferably formed using a deposition method that enables favorable step coverage. One such method is an atomic layer deposition (ALD) method. A material that can be formed by an ALD method is preferably used for the protective film. With the use of an ALD method, a dense protective film with reduced defects such as cracks and pinholes or with a uniform thickness can be formed. Furthermore, damage caused to a process member in forming the protective film can be reduced.

For example, by an ALD method, a uniform protective film with few defects can be formed even on a surface with a complex uneven shape or upper, side, and lower surfaces of a touch panel.

The light-emitting apparatus fabricated using the light-emitting device described in Embodiment 1 and Embodiment 2 can be obtained in the above manner.

The light-emitting apparatus in this embodiment uses the light-emitting device described in Embodiment 1 and Embodiment 2 and thus has favorable characteristics. Specifically, since the light-emitting device described in Embodiment 1 and Embodiment 2 is a light-emitting device with a long lifetime, the light-emitting apparatus can have favorable reliability. Furthermore, since the light-emitting apparatus using the light-emitting device described in Embodiment 1 and Embodiment 2 has favorable emission efficiency, the light-emitting apparatus can achieve low power consumption.

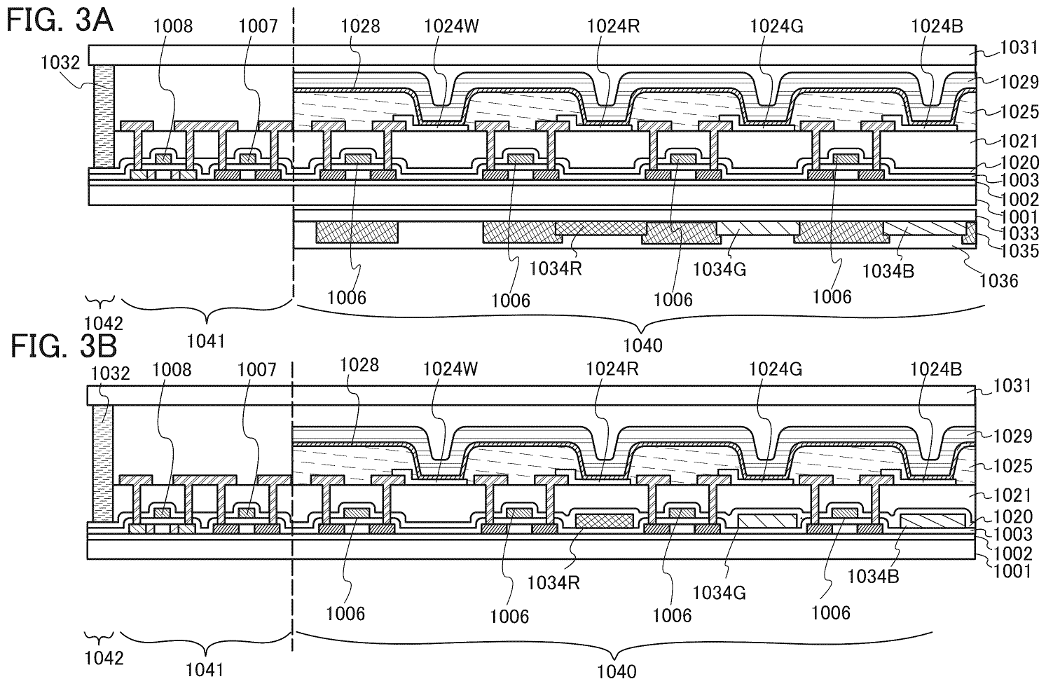

FIG. 3 shows examples of a light-emitting apparatus which achieves full color display by formation of a light-emitting device exhibiting white light emission and provision of coloring layers (color filters) and the like. FIG. 3A illustrates a substrate 1001, abase insulating film 1002, a gate insulating film 1003, gate electrodes 1006, 1007, and 1008, a first interlayer insulating film 1020, a second interlayer insulating film 1021, a peripheral portion 1042, a pixel portion 1040, a driver circuit portion 1041, anodes 1024W, 1024R, 1024G, and 1024B of the light-emitting devices, a partition 1025, an EL layer 1028, a second electrode 1029 of the light-emitting devices, a sealing substrate 1031, a sealant 1032, and the like.

In FIG. 3A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix is aligned and fixed to the substrate 1001. Note that the coloring layers and the black matrix 1035 are covered with an overcoat layer 1036. In FIG. 3A, there is a light-emitting layer from which light is extracted to the outside without passing through the coloring layers and a light-emitting layer from which light is extracted to the outside after passing through the coloring layers of the respective colors. The light that does not pass through the coloring layers is white, and the light that passes through the coloring layers is red, green, and blue, so that an image can be expressed with the pixels of four colors.

FIG. 3B shows an example in which the coloring layers (the red coloring layer 1034R, the green coloring layer 1034G, and the blue coloring layer 1034B) are formed between the gate insulating film 1003 and the first interlayer insulating film 1020. The coloring layers may be provided between the substrate 1001 and the sealing substrate 1031 in this manner.

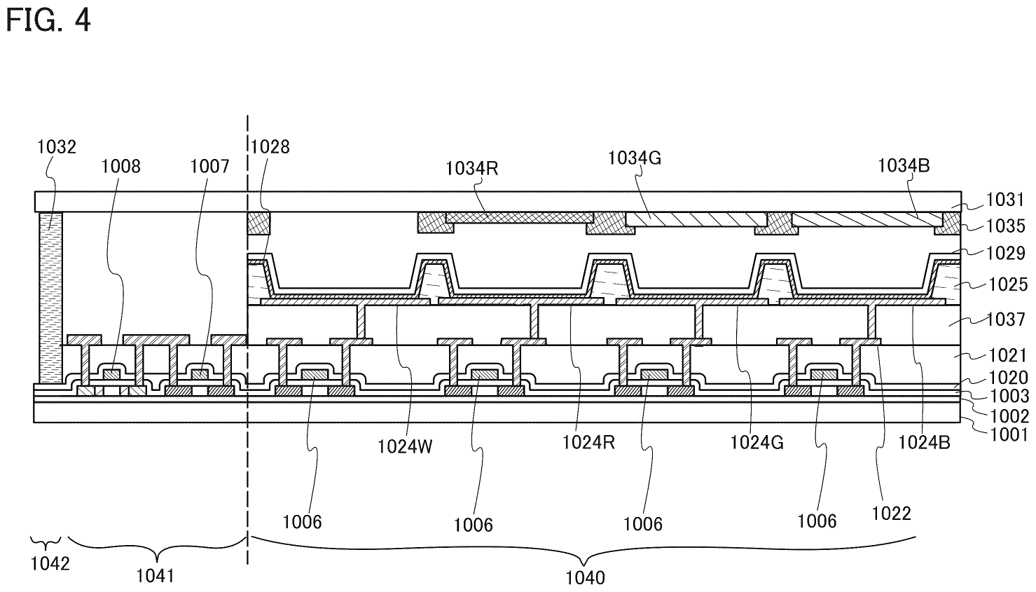

The above-described light-emitting apparatus is a light-emitting apparatus having a structure in which light is extracted to the substrate 1001 side where the FETs are formed (a bottom-emission type), but may be a light-emitting apparatus having a structure in which light emission is extracted to the sealing substrate 1031 side (a top-emission type). FIG. 4 shows a cross-sectional view of atop-emission light-emitting apparatus. In this case, a substrate that does not transmit light can be used as the substrate 1001. The top-emission light-emitting apparatus is formed in a manner similar to that of the bottom-emission light-emitting apparatus until a connection electrode which connects the FET and the anode of the light-emitting device is formed. Then, a third interlayer insulating film 1037 is formed to cover an electrode 1022. This insulating film may have a planarization function. The third interlayer insulating film 1037 can be formed using a material similar to that for the second interlayer insulating film or using any other known materials.

The anodes 1024W, 1024R, 1024G, and 1024B of the light-emitting devices may each be a cathode though they are anodes here. Furthermore, in the case of the top-emission light-emitting apparatus shown in FIG. 4, the anodes are preferably reflective electrodes. The EL layer 1028 has a structure similar to the structure of the EL layer 103 described in Embodiment 1 and Embodiment 2, with which white light emission can be obtained.

In the top-emission structure as shown in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 which is positioned between pixels. The coloring layers (the red coloring layer 1034R, the green coloring layer 1034G, and the blue coloring layer 1034B) and the black matrix may be covered with the overcoat layer. Note that a light-transmitting substrate is used as the sealing substrate 1031. Although an example in which full color display is performed using four colors of red, green, blue, and white is shown here, there is no particular limitation and full color display may be performed using four colors of red, yellow, green, and blue or three colors of red, green, and blue.

In the top-emission light-emitting apparatus, a microcavity structure can be favorably employed. A light-emitting device with a microcavity structure can be obtained with the use of a reflective electrode as the anode and a semi-transmissive and semi-reflective electrode as the cathode. At least an EL layer is provided between the reflective electrode and the semi-transmissive and semi-reflective electrode, and the EL layer includes at least a light-emitting layer functioning as a light-emitting region.

Note that the reflective electrode is a film having a visible light reflectance of 40% to 100%, preferably 70% to 100%, and a resistivity of 1×10⁻² Ωcm or lower. The semi-transmissive and semi-reflective electrode is a film having a visible light reflectance of 20% to 80%, preferably 40% to 70%, and a resistivity of 1×10⁻² Ωcm or lower.

Light emitted from the light-emitting layer included in the EL layer is reflected and resonated by the reflective electrode and the semi-transmissive and semi-reflective electrode.

In the light-emitting device, by changing the thicknesses of the transparent conductive film, the above-described composite material, the carrier-transport material, and the like, the optical path length between the reflective electrode and the semi-transmissive and semi-reflective electrode can be changed. Thus, light with a wavelength that is resonated between the reflective electrode and the semi-transmissive and semi-reflective electrode can be intensified while light with a wavelength that is not resonated therebetween can be attenuated.

Note that light that is reflected back by the reflective electrode (first reflected light) considerably interferes with light that directly enters the semi-transmissive and semi-reflective electrode from the light-emitting layer (first incident light); therefore, the optical path length between the reflective electrode and the light-emitting layer is preferably adjusted to (2n-1)k/4 (n is a natural number of 1 or larger and k is a wavelength of light emission to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer interposed between the EL layers.

With the microcavity structure, emission intensity with a particular wavelength in the front direction can be increased, whereby power consumption can be reduced. Note that in the case of a light-emitting apparatus which displays images with subpixels of four colors of red, yellow, green, and blue, the light-emitting apparatus can have favorable characteristics because the luminance can be increased owing to yellow light emission and each subpixel can employ a microcavity structure suitable for the wavelength of the corresponding color.

The light-emitting apparatus in this embodiment uses the light-emitting device described in Embodiment 1 and Embodiment 2 and thus has favorable characteristics. Specifically, a highly reliable light-emitting apparatus is achieved because the light-emitting device described in Embodiment 1 and Embodiment 2 is a light-emitting device with a long lifetime. Furthermore, since the light-emitting apparatus using the light-emitting device described in Embodiment 1 and Embodiment 2 has favorable emission efficiency, the light-emitting apparatus can achieve low power consumption.

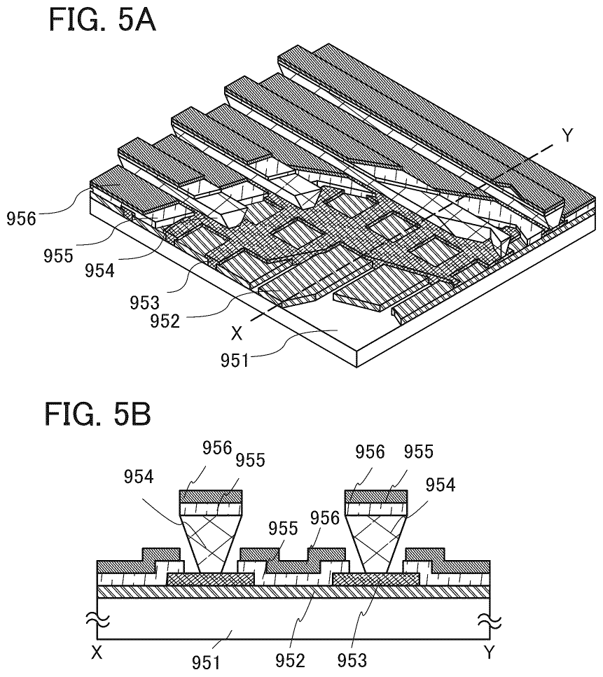

The active matrix light-emitting apparatus is described above, whereas a passive matrix light-emitting apparatus is described below. FIG. 5 illustrates a passive matrix light-emitting apparatus fabricated using the present invention. Note that FIG. 5A is a perspective view showing the light-emitting apparatus, and FIG. 5B is a cross-sectional view taken along the line X-Y in FIG. 5A. In FIG. 5, an EL layer 955 is provided between an electrode 952 and an electrode 956 over a substrate 951. An end portion of the electrode 952 is covered with an insulating layer 953. A partition layer 954 is provided over the insulating layer 953. Sidewalls of the partition layer 954 are aslope such that the distance between one sidewall and the other sidewall is gradually narrowed toward the surface of the substrate. That is, a cross section in the short side direction of the partition layer 954 is a trapezoidal shape, and the lower side (the side facing the same direction as the plane direction of the insulating layer 953 and touching the insulating layer 953) is shorter than the upper side (the side facing the same direction as the plane direction of the insulating layer 953, and not touching the insulating layer 953). Providing the partition layer 954 in this manner can prevent defects of the light-emitting device due to static charge or the like. The passive-matrix light-emitting apparatus also uses the light-emitting device described in Embodiment 1 and Embodiment 2; thus, the light-emitting apparatus can have favorable reliability or low power consumption.

Since many minute light-emitting devices arranged in a matrix can be controlled in the above-described light-emitting apparatus, the light-emitting apparatus can be suitably used as a display device for expressing images.

This embodiment can be freely combined with any of the other embodiments.

### Embodiment 4

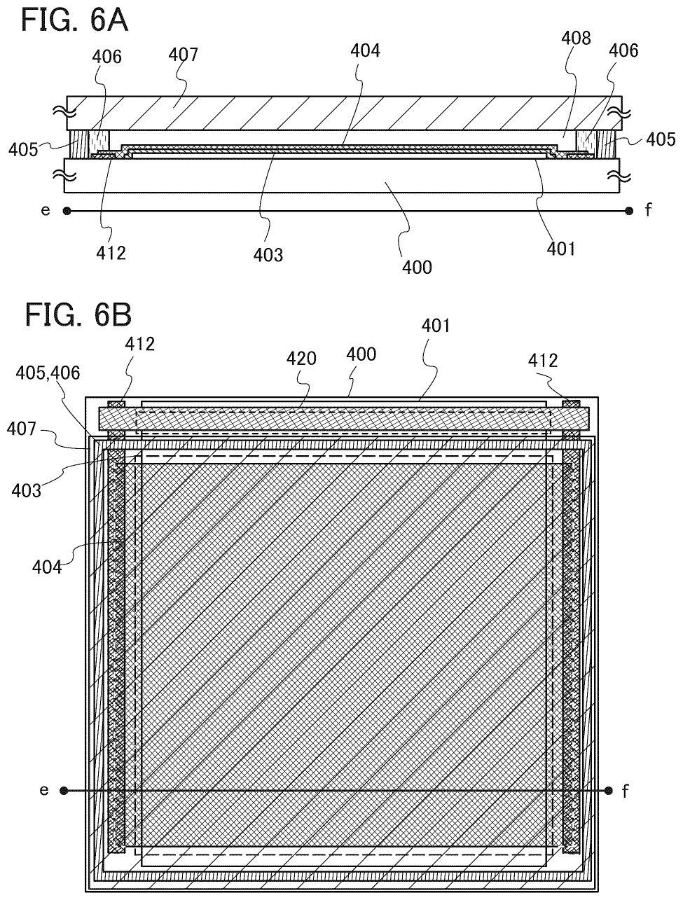

In this embodiment, an example in which the light-emitting device described in Embodiment 1 and Embodiment 2 is used for a lighting apparatus will be described with reference to FIG. 6. FIG. 6B is a top view of the lighting apparatus, and FIG. 6A is a cross-sectional view taken along the line e-f in FIG. 6B.

In the lighting apparatus in this embodiment, a first electrode 401 is formed over a substrate 400 which is a support and has a light-transmitting property. The first electrode 401 corresponds to the first electrode 101 in Embodiment 2. In the case where light emission is extracted from the first electrode 401 side, the first electrode 401 is formed using a material having a light-transmitting property.

A pad 412 for supplying a voltage to a second electrode 404 is formed over the substrate 400.

An EL layer 403 is formed over the first electrode 401. The EL layer 403 has a structure corresponding to that of the EL layer 103 in Embodiment 1 and Embodiment 2, or the structure in which the light-emitting units 511 and 512 are combined with the charge-generation layer 513. Note that for these structures, the corresponding description can be referred to.

The second electrode 404 is formed to cover the EL layer 403. The second electrode 404 corresponds to the second electrode 102 in Embodiment 2. In the case where light emission is extracted from the first electrode 401 side, the second electrode 404 is formed using a material having high reflectance. The second electrode 404 is supplied with a voltage when connected to the pad 412.

As described above, the lighting apparatus described in this embodiment includes a light-emitting device including the first electrode 401, the EL layer 403, and the second electrode 404. Since the light-emitting device is a light-emitting device with high emission efficiency, the lighting apparatus in this embodiment can be a lighting apparatus with low power consumption.

The substrate 400 over which the light-emitting device having the above structure is formed is fixed to a sealing substrate 407 with sealants 405 and 406 and sealing is performed, whereby the lighting apparatus is completed. It is possible to use only either the sealant 405 or 406. In addition, the inner sealant 406 (not illustrated in FIG. 6B) can be mixed with a desiccant, which enables moisture to be adsorbed, resulting in improved reliability.

When parts of the pad 412 and the first electrode 401 are provided to extend to the outside of the sealants 405 and 406, those can serve as external input terminals. An IC chip 420 or the like mounted with a converter or the like may be provided over the external input terminals.

The lighting apparatus described in this embodiment uses the light-emitting device described in Embodiment 1 and Embodiment 2 as an EL element; thus, the light-emitting apparatus can have favorable reliability at high temperatures. Furthermore, the light-emitting apparatus can have low power consumption.

### Embodiment 5

In this embodiment, examples of electronic apparatuses each partly including the light-emitting device described in Embodiment 1 and Embodiment 2 will be described. The light-emitting device described in Embodiment 1 and Embodiment 2 is a light-emitting device having a long lifetime and favorable reliability at high temperatures. As a result, the electronic apparatuses described in this embodiment can be electronic apparatuses each including a light-emitting portion with favorable reliability at high temperatures.

Examples of electronic apparatuses to which the light-emitting device is applied include television devices (also referred to as TV or television receivers), monitors for computers and the like, digital cameras, digital video cameras, digital photo frames, cellular phones (also referred to as portable telephones or portable telephone devices), portable game machines, portable information terminals, audio playback devices, and large game machines such as pin-ball machines. Specific examples of these electronic apparatuses are shown below.

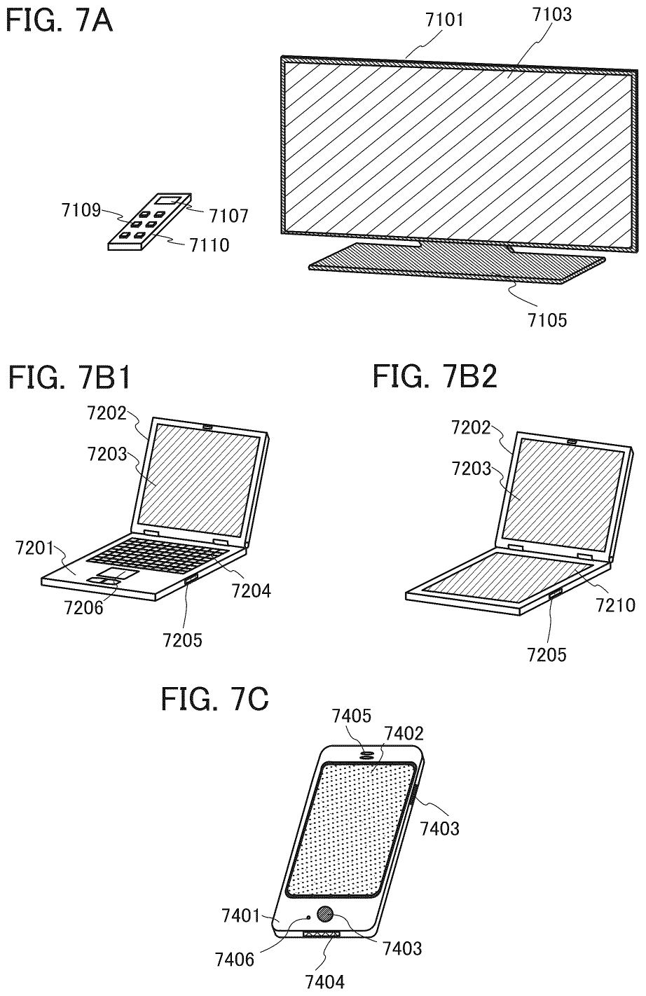

FIG. 7A shows an example of a television device. In the television device, a display portion 7103 is incorporated in a housing 7101. Here, a structure in which the housing 7101 is supported by a stand 7105 is shown. Images can be displayed on the display portion 7103, and the light-emitting devices described in Embodiment 1 and Embodiment 2 are arranged in a matrix in the display portion 7103.

The television device can be operated with an operation switch of the housing 7101 or a separate remote controller 7110. With operation keys 7109 of the remote controller 7110, channels and volume can be adjusted and images displayed on the display portion 7103 can be adjusted. Furthermore, a structure may be employed in which the remote controller 7110 is provided with a display portion 7107 for displaying data output from the remote controller 7110.

Note that the television device has a structure including a receiver, a modem, and the like. With the use of the receiver, a general television broadcast can be received, and when the television device is further connected to a communication network with or without a wire via the modem, one-way (from a sender to a receiver) or two-way (between a sender and a receiver or between receivers) data communication can be performed.

FIG. 7B1 is a computer which includes a main body 7201, a housing 7202, a display portion 7203, a keyboard 7204, an external connection port 7205, a pointing device 7206, and the like. Note that this computer is fabricated using the light-emitting devices described in Embodiment 1 and Embodiment 2 arranged in a matrix in the display portion 7203. The computer in FIG. 7B1 may be such a mode as in FIG. 7B2. The computer in FIG. 7B2 is provided with a second display portion 7210 instead of the keyboard 7204 and the pointing device 7206. The second display portion 7210 is of a touch-panel type, and input can be performed by adjusting display for input displayed on the second display portion 7210 with a finger or a dedicated pen. The second display portion 7210 can also display images other than the display for input. The display portion 7203 may also be a touch panel. Connecting the two screens with a hinge can prevent troubles such as a crack in or damage to the screens caused when the computer is stored or carried.

FIG. 7C shows an example of a portable terminal. A cellular phone includes operation buttons 7403, an external connection port 7404, a speaker 7405, a microphone 7406, and the like in addition to a display portion 7402 incorporated in a housing 7401. Note that the cellular phone includes the display portion 7402 which is fabricated by arranging the light-emitting devices described in Embodiment 1 and Embodiment 2 in a matrix.

The portable terminal shown in FIG. 7C may have a structure in which data can be input by touching the display portion 7402 with a finger or the like. In this case, operations such as making a call and creating an e-mail can be performed by touching the display portion 7402 with a finger or the like.

The display portion 7402 has mainly three screen modes. The first one is a display mode mainly for displaying images, and the second one is an input mode mainly for inputting data such as text. The third one is a display+input mode in which the two modes of the display mode and the input mode are combined.

For example, in the case of making a call or creating an e-mail, a text input mode mainly for inputting text is selected for the display portion 7402 so that an operation of inputting text displayed on the screen may be performed. In this case, it is preferable to display a keyboard or number buttons on almost the entire screen of the display portion 7402.

When a sensing device including a sensor for sensing inclination, such as a gyroscope sensor or an acceleration sensor, is provided inside the portable terminal, screen display of the display portion 7402 can be automatically changed by determining the orientation (vertical or horizontal) of the portable terminal.

The screen modes are changed by touching the display portion 7402 or operating the operation buttons 7403 of the housing 7401. Alternatively, the screen modes can be changed depending on the kind of image displayed on the display portion 7402. For example, when a signal of an image displayed on the display portion is moving image data, the screen mode is changed to the display mode, and when the signal is text data, the screen mode is changed to the input mode.

Moreover, in the input mode, when input by the touch operation of the display portion 7402 is not performed for a certain period while a signal sensed by an optical sensor in the display portion 7402 is sensed, the screen mode may be controlled so as to be changed from the input mode to the display mode.

The display portion 7402 can also function as an image sensor. For example, an image of a palm print, a fingerprint, or the like is taken when the display portion 7402 is touched with the palm or the finger, whereby personal authentication can be performed. Furthermore, when a backlight which emits near-infrared light or a sensing light source which emits near-infrared light is used in the display portion, an image of a finger vein, a palm vein, or the like can be taken.

Note that the structures described in this embodiment can be combined with any of the structures described Embodiment 1 to Embodiment 4 as appropriate.

As described above, the application range of the light-emitting apparatus including the light-emitting device described in Embodiment 1 and Embodiment 2 is so wide that this light-emitting apparatus can be applied to electronic apparatuses in a variety of fields. With the use of the light-emitting device described in Embodiment 1 and Embodiment 2, an electronic apparatus with high reliability at high temperatures can be obtained.

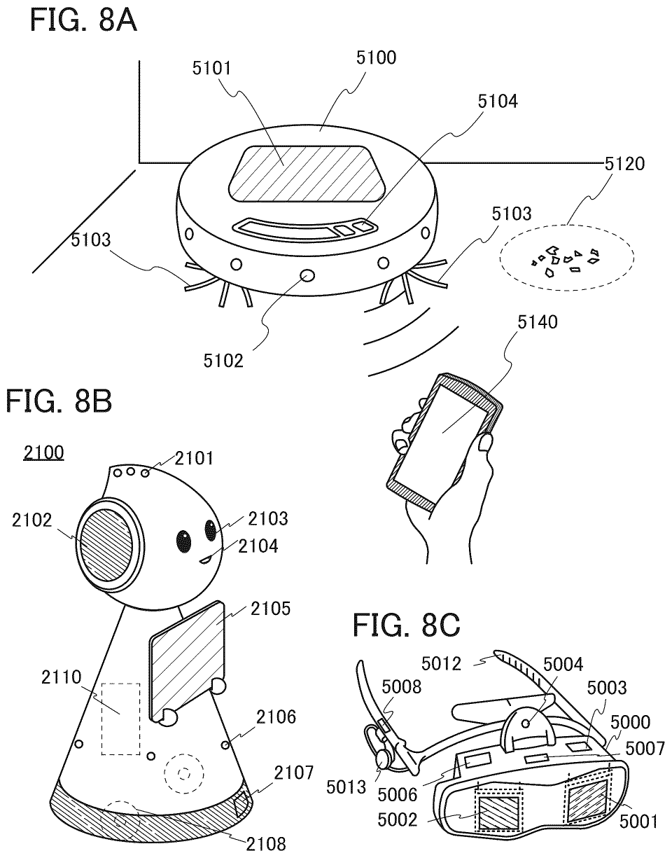

FIG. 8A is a schematic view showing an example of a cleaning robot.

A cleaning robot 5100 includes a display 5101 placed on its top surface, a plurality of cameras 5102 placed on its side surface, a brush 5103, and operation buttons 5104. Although not illustrated, the bottom surface of the cleaning robot 5100 is provided with a tire, an inlet, and the like. The cleaning robot 5100 also includes various sensors such as an infrared sensor, an ultrasonic sensor, an acceleration sensor, a piezoelectric sensor, an optical sensor, and a gyroscope sensor. In addition, the cleaning robot 5100 has a wireless communication means.

The cleaning robot 5100 is self-propelled, detects dust 5120, and sucks up the dust through the inlet provided on the bottom surface.

The cleaning robot 5100 can analyze images taken by the cameras 5102 to judge whether there is an obstacle such as a wall, furniture, or a step. When an object such as a wire that is likely to be caught in the brush 5103 is detected by image analysis, the rotation of the brush 5103 can be stopped.

The display 5101 can display the remaining capacity of a battery, the amount of vacuumed dust, and the like. The display 5101 may display a path on which the cleaning robot 5100 has run. The display 5101 may be a touch panel, and the operation buttons 5104 may be provided on the display 5101.

The cleaning robot 5100 can communicate with a portable electronic apparatus 5140 such as a smartphone. The portable electronic apparatus 5140 can display images taken by the cameras 5102. Accordingly, an owner of the cleaning robot 5100 can monitor the room even from the outside. The display on the display 5101 can be checked by the portable electronic apparatus such as a smartphone.

The light-emitting apparatus of one embodiment of the present invention can be used for the display 5101.

A robot 2100 shown in FIG. 8B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of detecting a speaking voice of a user, an environmental sound, and the like. The speaker 2104 also has a function of outputting sound. The robot 2100 can communicate with a user using the microphone 2102 and the speaker 2104.

The display 2105 has a function of displaying various kinds of data. The robot 2100 can display data desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect the presence of an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 8C shows an example of a goggle-type display. The goggle-type display includes, for example, a housing 5000, a display portion 5001, a speaker 5003, an LED lamp 5004, a connection terminal 5006, a sensor 5007 (a sensor having a function of measuring force, displacement, position, speed, acceleration, angular velocity, rotational frequency, distance, light, liquid, magnetism, temperature, chemical substance, sound, time, hardness, electric field, current, voltage, power, radiation, flow rate, humidity, gradient, oscillation, odor, or infrared rays), a microphone 5008, a display portion 5002, a support 5012, and an earphone 5013.

The light-emitting apparatus of one embodiment of the present invention can be used for the display portion 5001 and the display portion 5002.

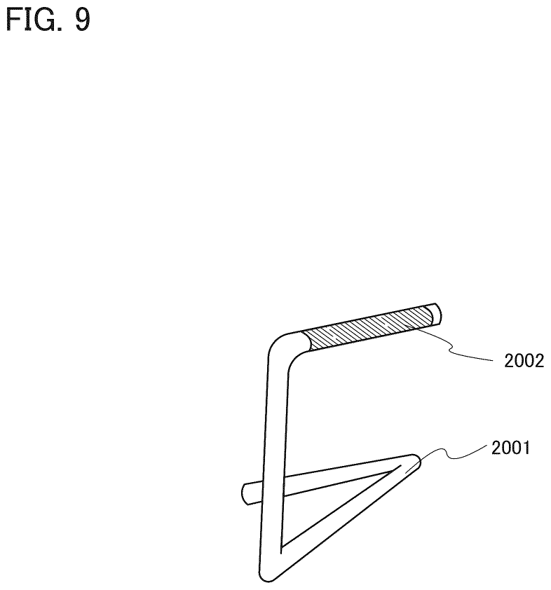

FIG. 9 shows an example in which the light-emitting device described in Embodiment 1 and Embodiment 2 is used for a table lamp which is a lighting apparatus. The table lamp shown in FIG. 9 includes a housing 2001 and a light source 2002, and the lighting apparatus described in Embodiment 3 may be used for the light source 2002.

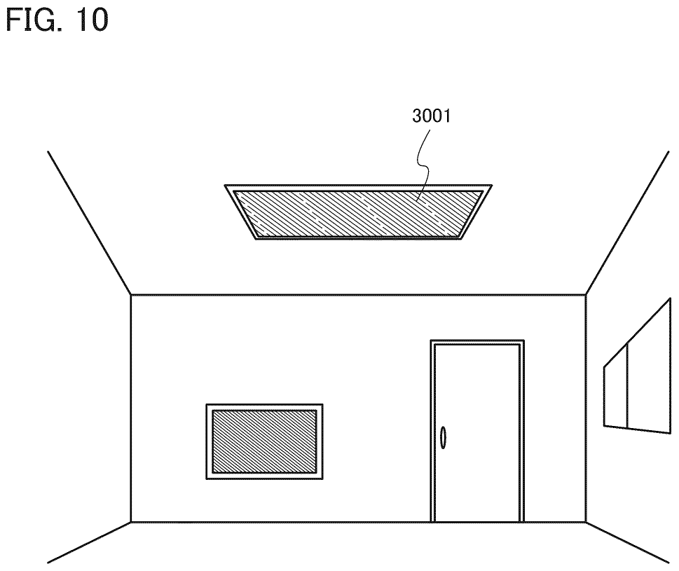

FIG. 10 shows an example in which the light-emitting device described in Embodiment 1 and Embodiment 2 is used for an indoor lighting apparatus 3001. Since the light-emitting device described in Embodiment 1 and Embodiment 2 is a light-emitting device having high reliability at high temperatures, the lighting apparatus can have high reliability at high temperatures. Furthermore, the light-emitting device described in Embodiment 1 and Embodiment 2 can have a larger area, and thus can be used for a large-area lighting apparatus. Furthermore, the light-emitting device described in Embodiment 1 and Embodiment 2 is thin, and thus can be used for a lighting apparatus having a reduced thickness.

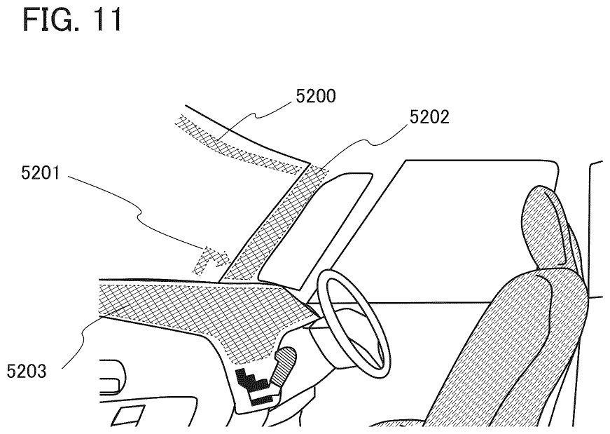

The light-emitting device described in Embodiment 1 and Embodiment 2 can also be incorporated in a windshield or a dashboard of an automobile. FIG. 11 shows one mode in which the light-emitting device described in Embodiment 1 and Embodiment 2 is used for a windshield and a dashboard of an automobile. The light-emitting device described in Embodiment 1 and Embodiment 2 is used for each of a display region 5200 to a display region 5203.

The display region 5200 and the display region 5201 are display devices which are provided in the automobile windshield and in which the light-emitting devices described in Embodiment 1 and Embodiment 2 are incorporated. When the light-emitting devices described in Embodiment 1 and Embodiment 2 are fabricated using electrodes having light-transmitting properties as an anode and a cathode, what is called see-through display devices, through which the opposite side can be seen, can be obtained. See-through display can be provided even in the automobile windshield without hindering the vision. Note that in the case where a driving transistor or the like is provided, it is preferable to use a transistor having a light-transmitting property, such as an organic transistor using an organic semiconductor material or a transistor using an oxide semiconductor.

The display region 5202 is a display device which is provided in a pillar portion and in which the light-emitting device described in Embodiment 1 and Embodiment 2 is incorporated. The display region 5202 can display an image taken by an imaging means provided on the automobile body to compensate for the view hindered by the pillar. Similarly, the display region 5203 provided in the dashboard portion can display an image taken by an imaging means provided on the outside of the automobile, so that the view hindered by the car body can be compensated for to avoid blind areas and enhance the safety. Showing an image so as to compensate for the area that cannot be seen makes it possible to confirm safety more naturally and comfortably.

The display region 5203 can provide a variety of kinds of data by displaying navigation data, a speedometer, a tachometer, a mileage, a fuel meter, a gearshift state, air-condition setting, and the like. The content or layout of the display can be changed freely in accordance with the preference of a user. Note that such data can also be displayed on the display region 5200 to the display region 5202. The display region 5200 to the display region 5203 can also be used as lighting apparatuses.

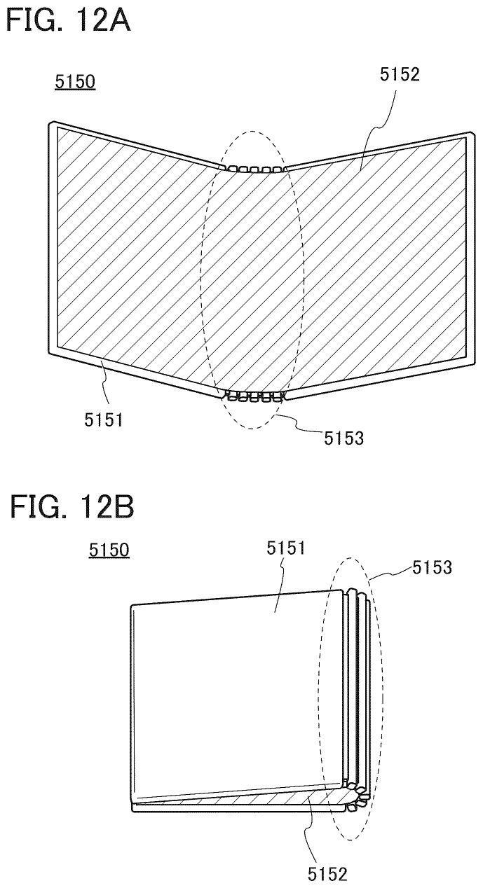

FIG. 12A and FIG. 12B show a foldable portable information terminal 5150. The foldable portable information terminal 5150 includes a housing 5151, a display region 5152, and a bend portion 5153. FIG. 12A shows the portable information terminal 5150 that is opened. FIG. 12B shows the portable information terminal that is folded. The portable information terminal 5150 is compact in size and has excellent portability when folded, despite its large display region 5152.

The display region 5152 can be folded in half with the bend portion 5153. The bend portion 5153 is formed of a stretchable member and a plurality of supporting members. When the display region is folded, the stretchable member stretches and the bend portion 5153 is folded with a curvature radius of 2 mm or more, preferably 3 mm or more.

Note that the display region 5152 may be a touch panel (an input/output device) including a touch sensor (an input device). The light-emitting apparatus of one embodiment of the present invention can be used for the display region 5152.

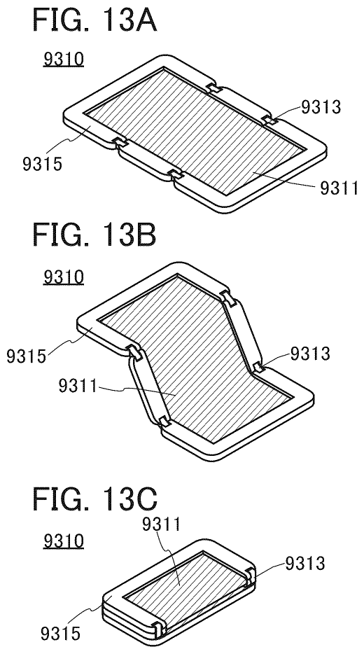

FIG. 13A to FIG. 13C show a foldable portable information terminal 9310. FIG. 13A shows the portable information terminal 9310 that is opened. FIG. 13B shows the portable information terminal 9310 which is in the state of being changed from one of an opened state and a folded state to the other. FIG. 13C shows the portable information terminal 9310 that is folded. The portable information terminal 9310 is excellent in portability when folded, and is excellent in display browsability when opened because of a seamless large display region.

A display panel 9311 is supported by three housings 9315 joined together by hinges 9313. Note that the display panel 9311 may be a touch panel (an input/output device) including a touch sensor (an input device). By folding the display panel 9311 at the hinges 9313 between two housings 9315, the portable information terminal 9310 can be reversibly changed in shape from the opened state to the folded state. The light-emitting apparatus of one embodiment of the present invention can be used for the display panel 9311.

### Example 1

In this example, a synthesis method of 2,9-di(1-naphthyl)-10-phenylanthracene (abbreviation: 2αN-αNPhA), which is an anthracene compound for a host material of one embodiment of the present invention, will be described in detail. The structural formula of 2αN-αNPhA is shown below.

### [Chemical Formula 11]

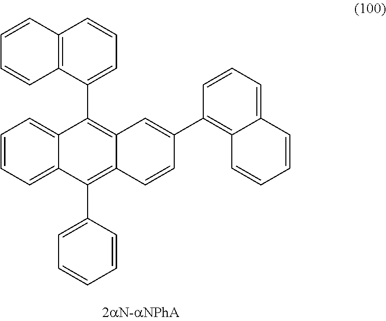 [SMILES: c1ccc(-c2c3ccccc3c(-c3cccc4ccccc34)c3cc(-c4cccc5ccccc45)ccc23)cc1]

Into a 200 mL three-necked flask were put 1.1 g (2.7 mmol) of 2-chloro-9-(1-naphthyl)-10-phenylanthracene, 0.93 g (5.4 mmol) of 1-naphthylboronic acid, 0.11 g (0.30 mmol) of di(1-adamanthyl)-n-butylphosphine, 1.9 g (9.0 mmol) of tripotassium phosphate, and 0.67 g (9.0 mmol) of tert-butyl alcohol, and the air in the flask was replaced with nitrogen. To the mixture was added 14 mL of diethylene glycol dimethyl ether, and the mixture was degassed by being stirred under reduced pressure. To this mixture was added 34 mg (0.15 mmol) of palladium(II) acetate, and the mixture was stirred at 130° C. under a nitrogen stream for 12 hours.

After the stirring, water was added to this mixture, the solid obtained by performing suction filtration was dissolved in toluene, and the solution was subjected to suction filtration through Celite (Wako Pure Chemical Industries, Ltd., Catalog No. 531-16855 (the same applies hereinafter)), alumina, and Florisil (Wako Pure Chemical Industries, Ltd., Catalog No. 540-00135 (the same applies hereinafter)). The solid obtained by concentrating the obtained filtrate was purified by high-performance liquid chromatography (HPLC) and then recrystallized with toluene to give 1.0 g of a target pale yellow solid at a yield of 73%. The synthesis scheme of this synthesis method is shown below.

### [Chemical Formula 12]

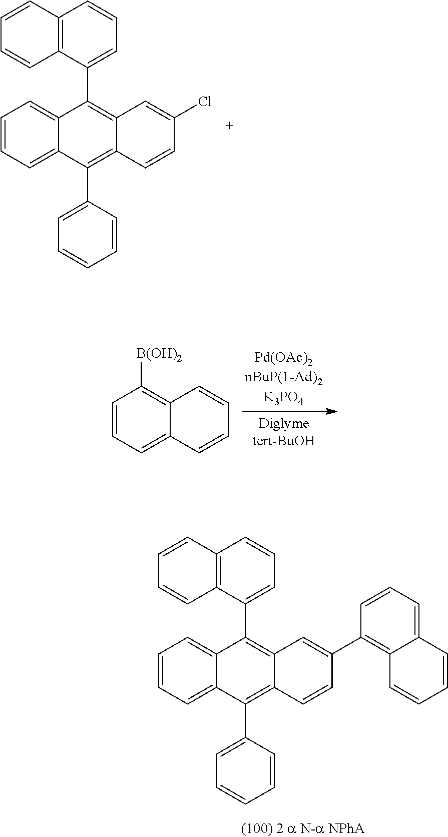

By the train sublimation method, 1.0 g of the obtained pale yellow solid was sublimated and purified. The sublimation purification was performed by heating the pale yellow solid at 220° C. under the conditions where the pressure was 3.8 Pa and the argon flow rate was 5.0 mL/min. After the sublimation purification, 0.92 g of a pale yellow solid was obtained at a collection rate of 92%.

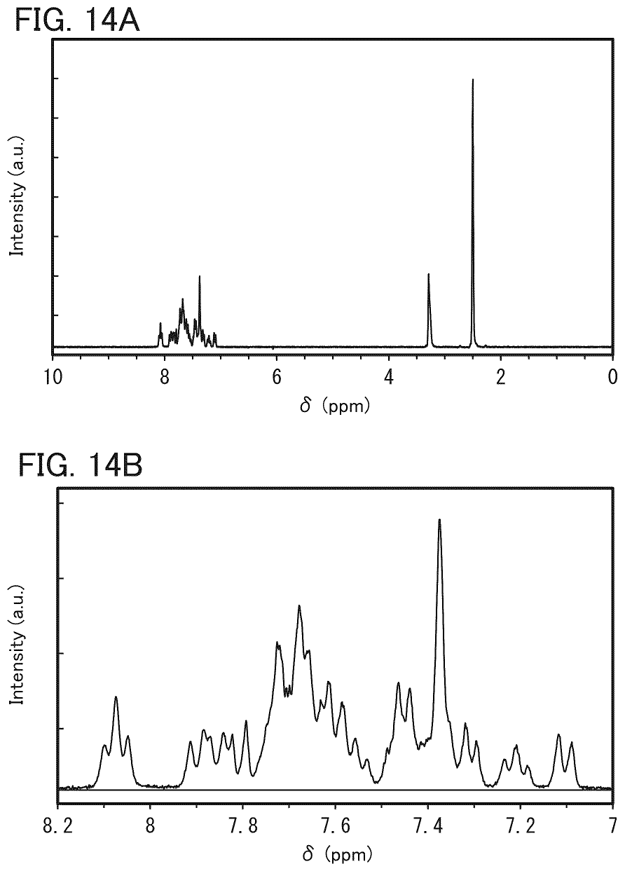

Analysis results of the obtained pale yellow solid by nuclear magnetic resonance spectroscopy (¹H-NMR) are shown below. FIG. 14A and FIG. 14B show ¹H-NMR charts. Note that FIG. 14B is a chart showing an enlarged view of a range of 7.0 ppm to 8.2 ppm in FIG. 14A. These results revealed that 2αN-αNPhA, which is the organic compound of one embodiment of the present invention represented by Structural Formula (100) above, was obtained in this example.

¹H NMR (DMSO-d₆, 300 MHz): δ=7.10 (d, J=8.7 Hz, 1H), 7.21 (t, J=7.5 Hz, 1H), 7.30-7.91 (m, 22H), 8.05-8.10 (m, 2H).

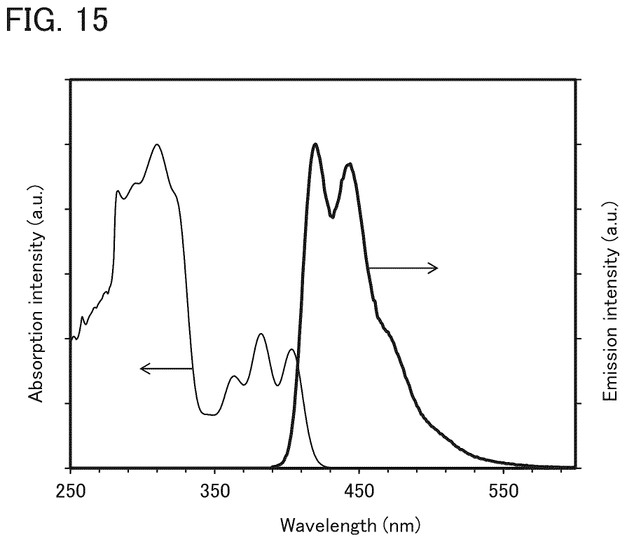

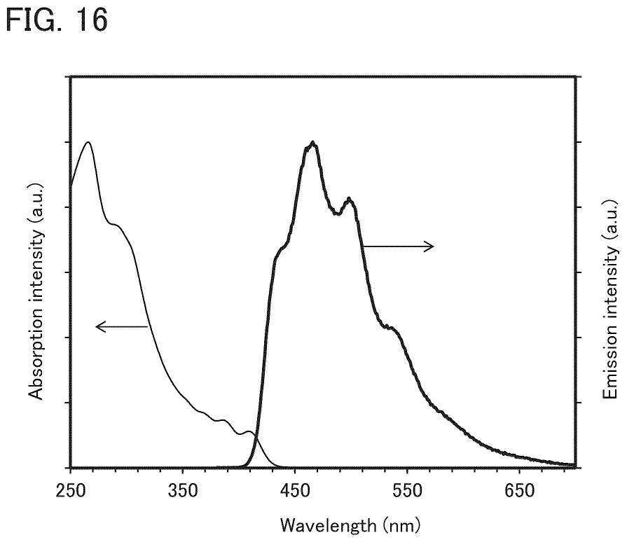

Next, FIG. 15 shows the measurement results of the absorption spectrum and the emission spectrum of 2αN-αNPhA in a toluene solution. FIG. 16 shows the absorption spectrum and the emission spectrum of a thin film. The solid thin film of 2αN-αNPhA was fabricated over a quartz substrate by a vacuum evaporation method. The absorption spectrum of 2αN-αNPhA in the toluene solution was calculated by measurement with an ultraviolet-visible light spectrophotometer (V550, manufactured by JASCO Corporation) and subtraction of the spectrum of toluene alone in a quartz cell. The absorption spectrum of the thin film of 2αN-αNPhA was calculated from the absorbance (−log₁₀[% T/(100−% R)], which was obtained from the transmittance and reflectance including the substrate by measurement with a spectrophotometer (U-4100 Spectrophotometer, manufactured by Hitachi High-Technologies Corporation). The emission spectrum was measured with a fluorescence spectrophotometer (FS920, manufactured by Hamamatsu Photonics K.K.).

The toluene solution of 2αN-αNPhA exhibited absorption peaks at around 403 nm, 382 nm, 363 nm, 310 nm, and 283 nm, and emission wavelength peaks at around 443 nm and 420 nm (excitation wavelength: 382 nm). The solid thin film of 2αN-αNPhA exhibited absorption peaks at around 409 nm, 387 nm, 367 nm, 291 nm, and 266 nm, and emission wavelength peaks at around 536 nm, 498 nm, 466 nm, and 440 nm (excitation wavelength: 370 nm).

Note that 2αN-αNPhA was confirmed to emit blue light. It was found that 2αN-αNPhA can be used as a host of an emission substance or a substance that emits fluorescence in a visible region. Furthermore, the thin film of 2αN-αNPhA was found to have a good film quality with difficulty in aggregation and little change in shape even under the air.

Next, the HOMO level and the LUMO level of 2αN-αNPhA were calculated on the basis of a cyclic voltammetry (CV) measurement. The calculation method is shown below. An electrochemical analyzer (model number: ALS model 600A or 600C, manufactured by BAS Inc.) was used as a measurement apparatus. To prepare a solution for the CV measurement, dehydrated dimethylformamide (DMF) (manufactured by Sigma-Aldrich Inc., 99.8%, catalog No. 22705-6) was used as a solvent, tetra-n-butylammonium perchlorate (n-Bu₄NClO₄) (manufactured by Tokyo Chemical Industry Co., Ltd., catalog No. T0836) as a supporting electrolyte was dissolved at a concentration of 100 mmol/L, and the object to be measured was dissolved at a concentration of 2 mmol/L.

A platinum electrode (PTE platinum electrode, manufactured by BAS Inc.) was used as a working electrode, another platinum electrode (Pt counter electrode for VC-3 (5 cm), manufactured by BAS Inc.) was used as an auxiliary electrode, and an Ag/Ag⁺ electrode (RE7 reference electrode for non-aqueous solvent, manufactured by BAS Inc.) was used as a reference electrode. Note that the measurement was performed at room temperature (20 to 25° C.).

The scan speed in the CV measurement was fixed to 0.1 V/sec, and an oxidation potential Ea [V] and a reduction potential Ec [V] with respect to the reference electrode were measured. Ea is an intermediate potential of an oxidation-reduction wave, and Ec is an intermediate potential of a reduction-oxidation wave. Here, since the potential energy of the reference electrode used in this example with respect to the vacuum level is known to be −4.94 [eV], the HOMO level and the LUMO level can be calculated by the following formulae: HOMO level [eV]−4.94−Ea and LUMO level [eV]=−4.94−Ec.

As a result, the HOMO level was found to be −5.81 eV in the measurement of the oxidation potential Ea [V] of 2αN-αNPhA. In contrast, the LUMO level was found to be −2.79 eV in the measurement of the reduction potential Ec [V].

### Example 2

In this example, a synthesis method of 9-(1-naphthyl)-10-phenyl-2-(5-phenyl-1-naphthyl)anthracene (abbreviation: 2PαN-αNPhA), which is an anthracene compound for a host material of one embodiment of the present invention, will be described in detail. The structural formula of 2PαN-αNPhA is shown below.

### [Chemical Formula 13]

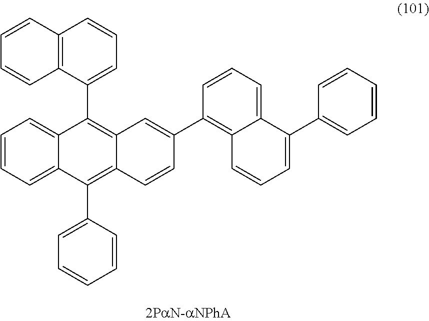 [SMILES: c1ccc(-c2cccc3c(-c4ccc5c(-c6ccccc6)c6ccccc6c(-c6cccc7ccccc67)c5c4)cccc23)cc1]

Into a 200 mL three-necked flask were put 1.3 g (3.0 mmol) of 2-chloro-9-(1-naphthyl)-10-phenylanthracene, 1.2 g (3.7 mmol) of 2-(5-phenyl-1-naphthyl)-4,4,5,5-tetramethyl-1,3,2-dioxaborolan, 0.13 g (0.36 mmol) of di(1-adamanthyl)-n-butylphosphine, 2.0 g (9.2 mmol) of tripotassium phosphate, 0.68 g (9.1 mmol) of tert-butyl alcohol, and 15 mL of diethylene glycol dimethyl ether, and the mixture was degassed by being stirred under reduced pressure. To this mixture was added 37 mg (0.17 mmol) of palladium(II) acetate, and the mixture was stirred at 130° C. under a nitrogen stream for 8 hours. After the stirring, water was added to this mixture, and a precipitated solid was collected by suction filtration. The obtained solid was purified by silica gel column chromatography (toluene:hexane=1:4) and then purified by high-performance liquid chromatography (HPLC) to give a solid. The obtained solid was recrystallized with toluene to give 0.95 g of a target white powder at a yield of 54%. The synthesis scheme of this synthesis method is shown below.

### [Chemical Formula 14]

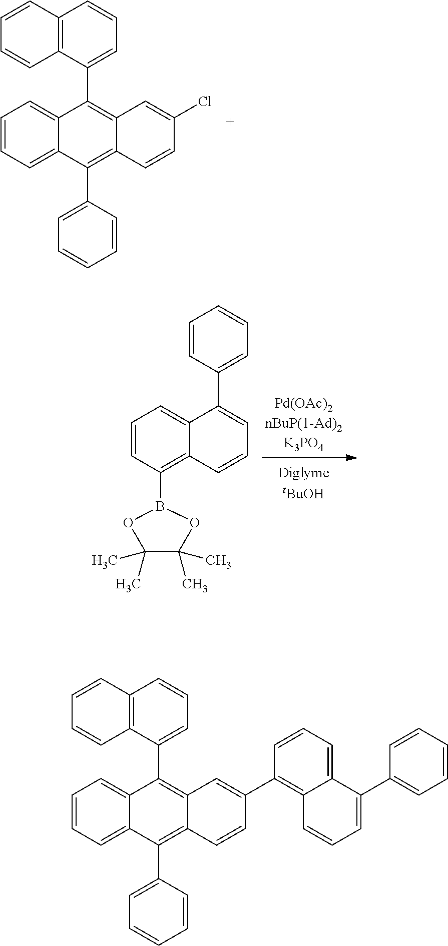

By the train sublimation method, 0.95 g of the obtained white powder was sublimated and purified. The sublimation purification was performed by heating the white powder at 275° C. for 18 hours under the conditions where the pressure was 3.4 Pa and the argon flow rate was 10 mL/min. After the sublimation purification, 0.72 g of a pale yellow powder was obtained at a collection rate of 76%.

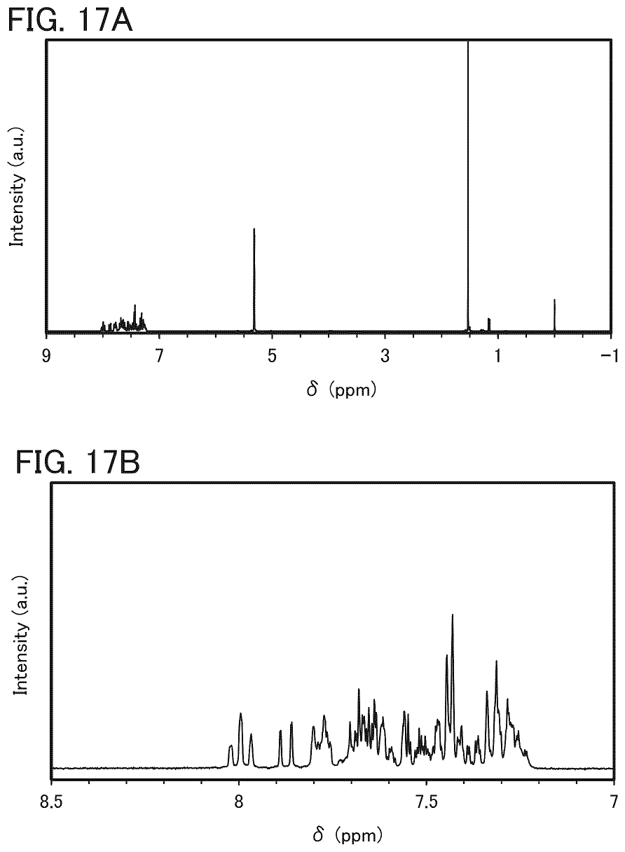

Analysis results of the obtained pale yellow powder by nuclear magnetic resonance spectroscopy (¹H-NMR) are shown below. FIG. 17A and FIG. 17B show ¹H-NMR charts. Note that FIG. 17B is a chart showing an enlarged view of a range of 7.0 ppm to 8.5 ppm in FIG. 17A. These results revealed that 2PαN-αNPhA, which is represented by Structural Formula (101) above, was obtained in this example.

¹H NMR (CD₂Cl₂,300 MHz): δ=7.23-7.80 (m, 27H), 7.88 (dd, J=9.0 Hz, 0.9 Hz, 1H), 7.97-8.02 (m, 2H).

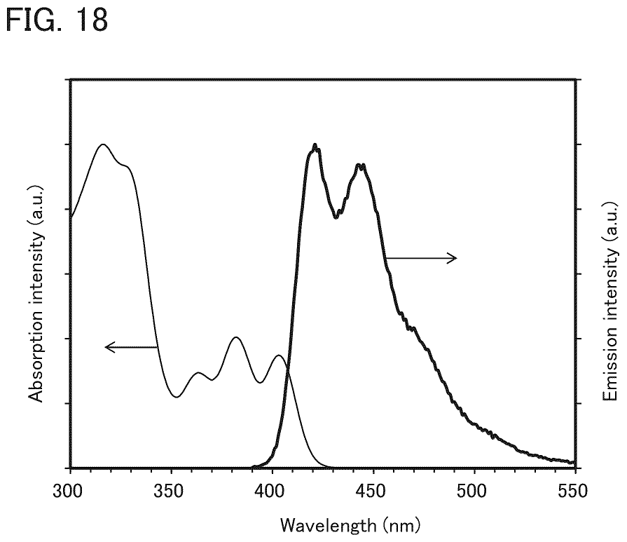

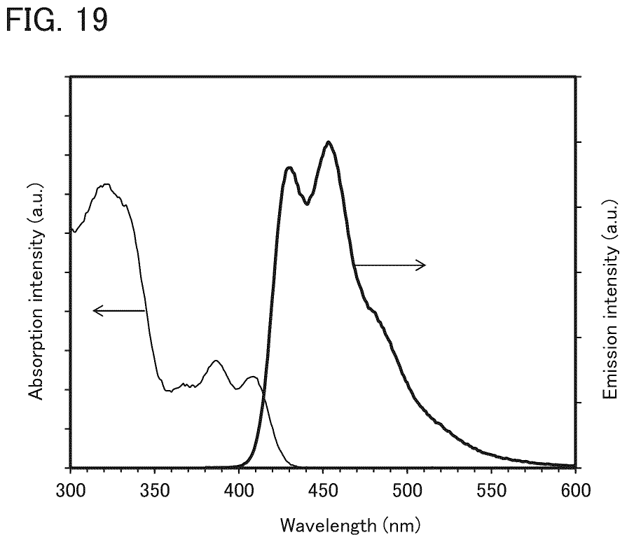

Next, FIG. 18 and FIG. 19 show the measurement results of the absorption spectrum and the emission spectrum of 2PαN-αNPhA in a toluene solution. The measurement was performed in a manner similar to that in Example 1.

According to FIG. 18, absorption peaks were observed at around 403 nm, 382 nm, 363 nm, and 316 nm, and emission wavelength peaks were observed at around 421 nm and 443 nm (excitation wavelength: 382 nm). According to the results in FIG. 19, the solid thin film of 2PαN-αNPhA exhibited absorption peaks at around 405 nm, 386 nm, 367 nm, 333 nm, and 321 nm, and emission wavelength peaks at around 430 nm and 453 nm (excitation wavelength: 370 nm).

Note that 2PαN-αNPhA was confirmed to emit blue light. The organic compound of one embodiment of the present invention, 2PαN-αNPhA, can be used as a host of an emission substance or a substance that emits fluorescence in a visible region. Furthermore, the thin film of 2PαN-αNPhA was found to have a good film quality with difficulty in aggregation and little change in shape even under the air.

Next, the HOMO level and the LUMO level of 2PαN-αNPhA were calculated on the basis of a cyclic voltammetry (CV) measurement. The calculation method is similar to that described in Example 1.

As a result, the HOMO level was found to be −5.86 eV in the measurement of the oxidation potential Ea [V] of 2PαN-αNPhA. In contrast, the LUMO level was found to be −2.80 eV in the measurement of the reduction potential Ec [V].

### Example 3

Described in this example is the light-emitting device 1, which uses as a host material the anthracene compound for a host material of one embodiment of the present invention described in Embodiment 1. Also shown are the comparative light-emitting device 1 and the comparative light-emitting device 2, each of which uses as a host material an organic compound having a structure similar to that of the anthracene compound for a host material of one embodiment of the present invention. Structural formulae of organic compounds used for the light-emitting device 1, the comparative light-emitting device 1, and the comparative light-emitting device 2 are shown below.

### [Chemical Formula 15]

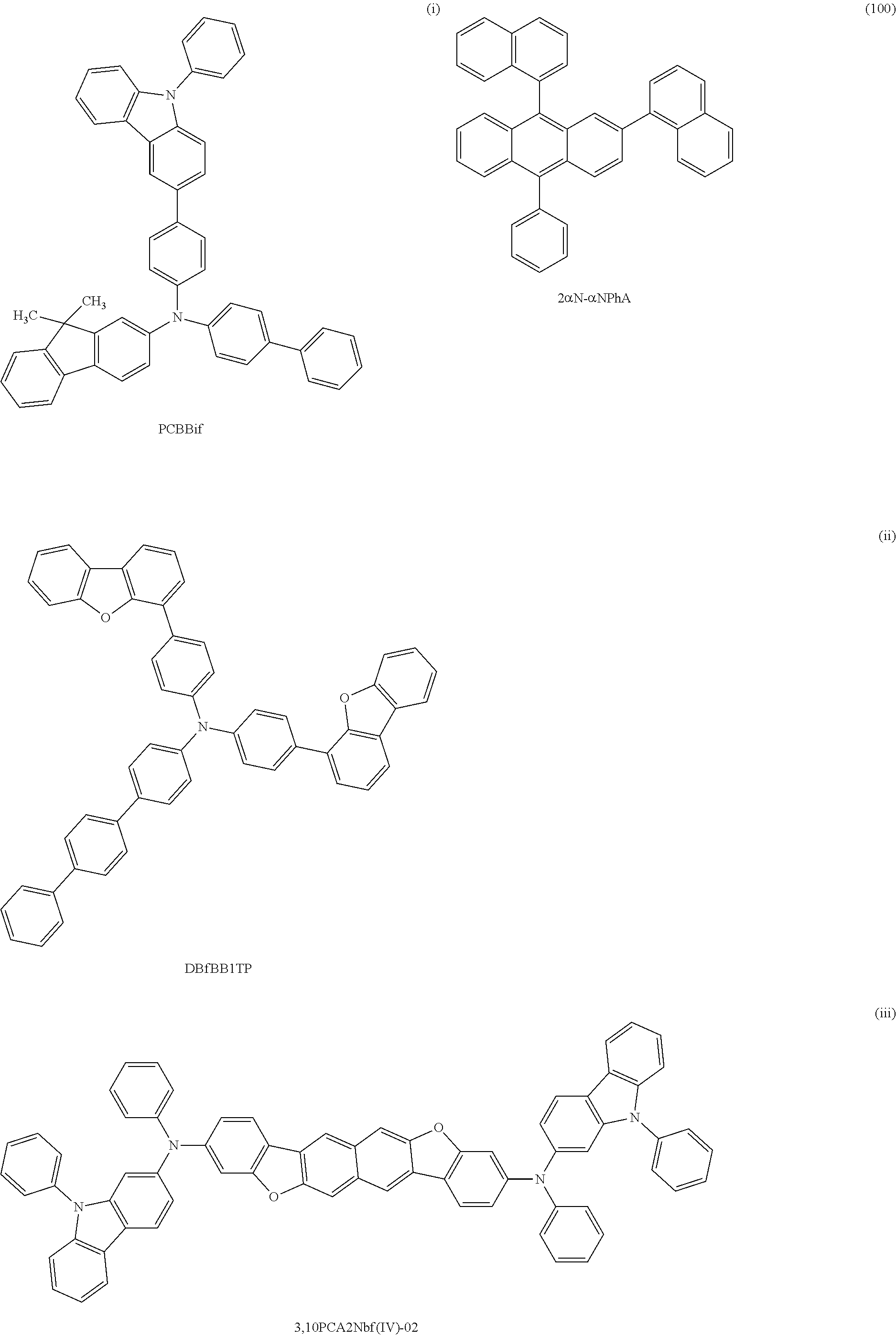

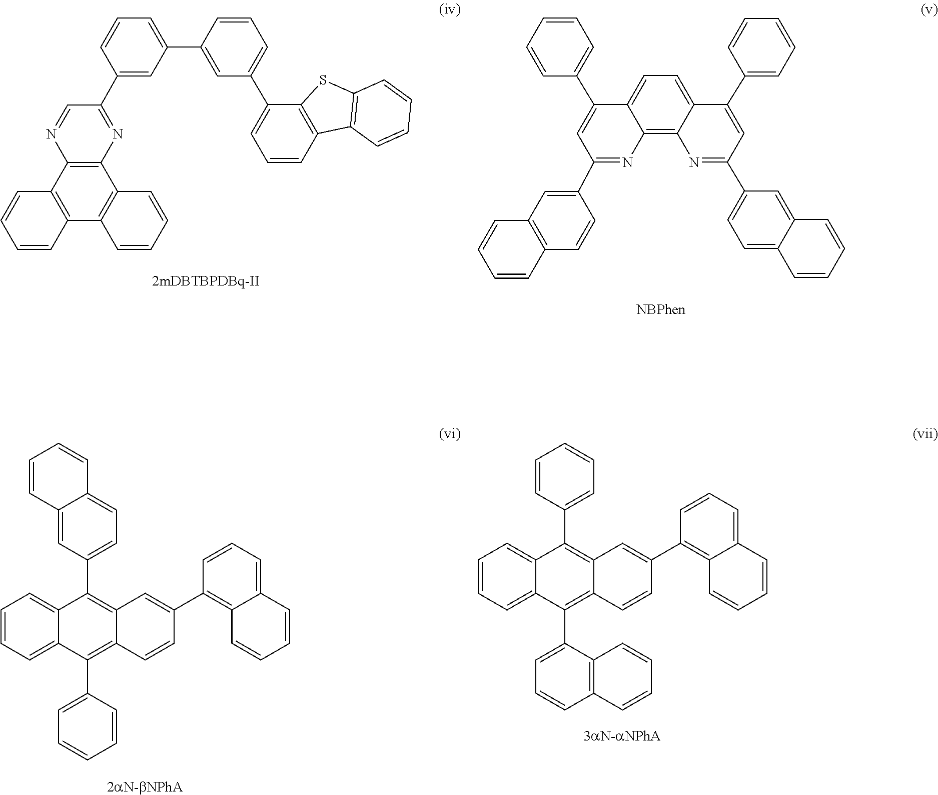

### (Fabrication Method of Light-Emitting Device 1)

First, a film of indium tin oxide containing silicon oxide (ITSO) was formed over a glass substrate by a sputtering method, so that the first electrode 101 was formed. Note that the film thickness was 70 nm and the area of the electrode was 2 mm×2 mm.

Next, in pretreatment for forming the light-emitting device over the substrate, the surface of the substrate was washed with water and baked at 200° C. for one hour, and then UV ozone treatment was performed for 370 seconds.

After that, the substrate was transferred into a vacuum evaporation apparatus in which the pressure was reduced to approximately 10-4 Pa, vacuum baking at 170° C. for 30 minutes was performed in a heating chamber in the vacuum evaporation apparatus, and then the substrate was cooled down for approximately 30 minutes.

Next, the substrate over which the first electrode 101 was formed was fixed to a substrate holder provided in the vacuum evaporation apparatus so that the surface over which the first electrode 101 was formed faced downward, and N-(1,1′-biphenyl-4-yl)-N-[4-(9-phenyl-9H-carbazol-3-yl)phenyl]-9,9-dimethyl-9H-fluoren-2-amine (abbreviation: PCBBiF) represented by Structural Formula (i) above and ALD-MP001Q (produced by Analysis Atelier Corporation, material serial No. 1S20170124) were co-evaporated over the first electrode 101 to have a weight ratio of 1:0.1 (=PCBBiF: ALD-MP001Q) to a thickness of 10 nm by an evaporation method using resistance heating, whereby the hole-injection layer 111 was formed.

Next, as the first hole-transport layer 112-1, PCBBiF was deposited by evaporation to a thickness of 20 nm over the hole-injection layer 111, and then, as the second hole-transport layer 112-2, N,N-bis[4-(dibenzofuran-4-yl)phenyl]-4-amino-p-terphenyl (abbreviation: DBfBB1TP) represented by Structural Formula (ii) above was deposited by evaporation to a thickness of 10 nm, whereby the hole-transport layer 112 was formed. Note that the second hole-transport layer 112-2 also functions as an electron-blocking layer.

Subsequently, 2,9-di(1-naphthyl)-10-phenylanthracene (abbreviation: 2αN-αNPhA) represented by Structural Formula (100) above, and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iii) above were deposited by co-evaporation to a thickness of 25 nm at a weight ratio of 1:0.015 (=2αN-αNPhA: 3,10PCA2Nbf(IV)-02), so that the light-emitting layer 113 was formed.

After that, over the light-emitting layer 113, 2-[3′-(dibenzothiophen-4-yl)biphenyl-3-yl]dibenzo[f,h]quinoxaline (abbreviation: 2mDBTBPDBq-II) represented by Structural Formula (iv) above was deposited by evaporation to a thickness of 15 nm, and then, 2,9-di(2-naphthyl)-4,7-diphenyl-1,10-phenanthroline (abbreviation: NBPhen) represented by Structural Formula (v) above was deposited by evaporation to a thickness of 10 nm, so that the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 1 of this example was fabricated.

### (Fabrication Method of Comparative Light-Emitting Device 1)

The comparative light-emitting device 1 was fabricated in a manner similar to that for the light-emitting device 1 except that 2αN-αNPhA in the light-emitting device 1 was replaced with 2-(1-naphthyl)-9-(2-naphthyl)-10-phenylanthracene (abbreviation: 2αN-QNPhA) represented by Structural Formula (vi) above.

### (Fabrication Method of Comparative Light-Emitting Device 2)

The comparative light-emitting device 2 was fabricated in a manner similar to that for the light-emitting device 1 except that 2αN-αNPhA in the light-emitting device 1 was replaced with 2,10-di(1-naphthyl)-9-phenylanthracene (abbreviation: 3αN-αNPhA) represented by Structural Formula (vii) above.

The device structures of the light-emitting device 1, the comparative light-emitting device 1, and the comparative light-emitting device 2 are listed in the following table.

TABLE 1

| | Hole- | | | Light- | | | Electron- |
|---|---|---|---|---|---|---|---|
| | injection | Hole-transport layer | | emitting | | | injection |
| | layer | 1 | 2 | layer | Electron-transport layer | | layer |
| | 10 nm | 20 nm | 10 nm | 25 nm | 15 nm | 10 nm | 1 nm |
| Light-emitting | PCBBIF: | PCBBiF | DBfBB1TP | *1 | 2mDBTBPDBq-II | NBPhen | LiF |
| device 1 | ALD- | | | | | | |
| Comparative light- | MP001Q | | | *2 | | | |
| emitting device 1 | (1:0.1) | | | | | | |
| Comparative light- | | | | *3 | | | |
| emitting device 2 | | | | | | | |

*1 2αN-αNPhA:3,10PCA2Nbf(IV)-02(1:0.015)
*2 2αN-βNPhA:3,10PCA2Nbf(IV)-02(1:0.015)
*1 3αN-αNPhA:3,10PCA2Nbf(IV)-02(1:0.015)

These light-emitting devices were subjected to sealing with a glass substrate (a sealant was applied to surround the devices, followed by UV treatment and one-hour heat treatment at 80° C. at the time of sealing) in a glove box containing a nitrogen atmosphere so that the light-emitting devices are not exposed to the air. Then, the initial characteristics and reliability of these light-emitting devices were measured. Note that the measurement was performed at room temperature.

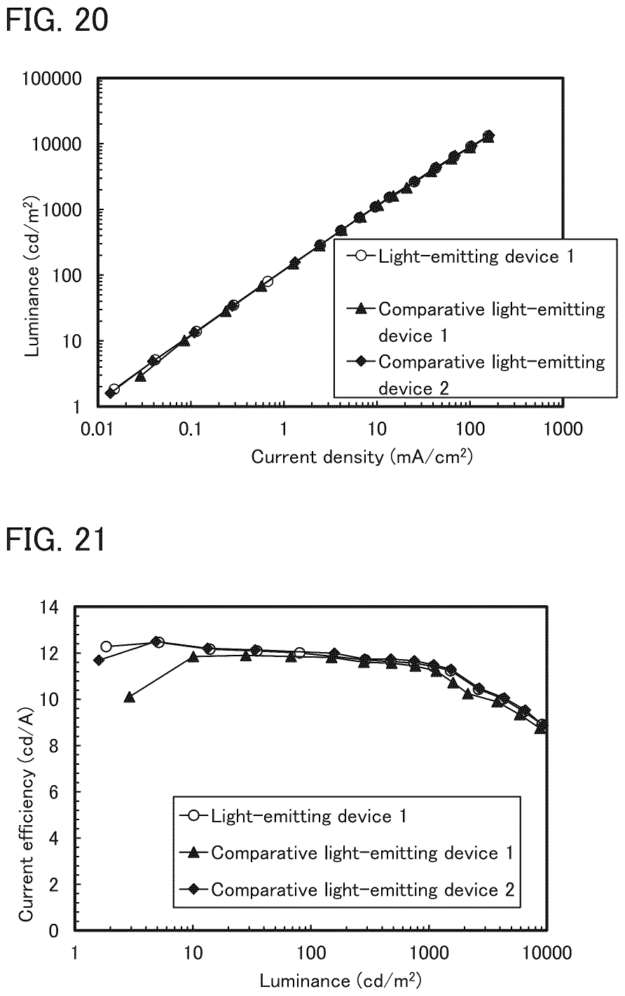

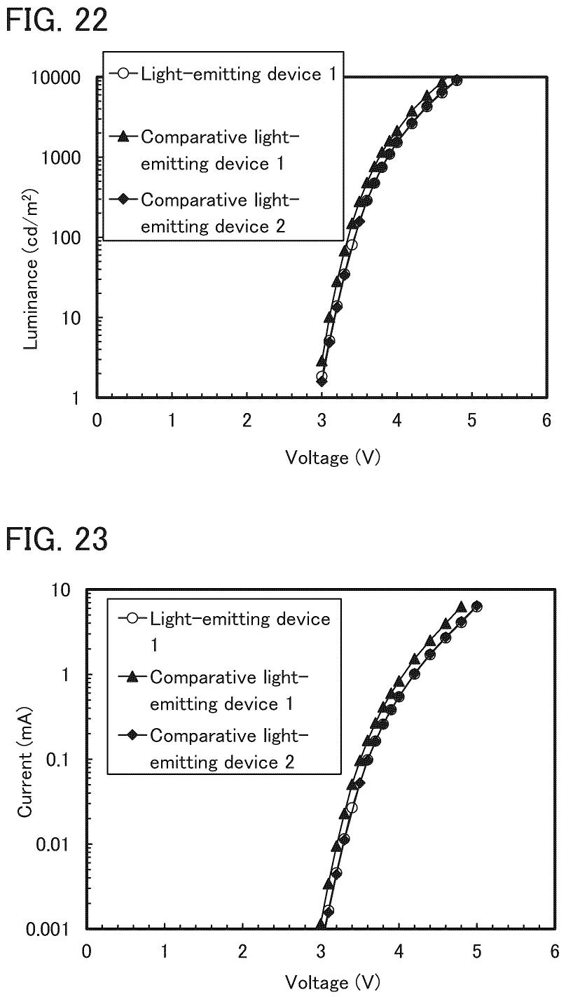

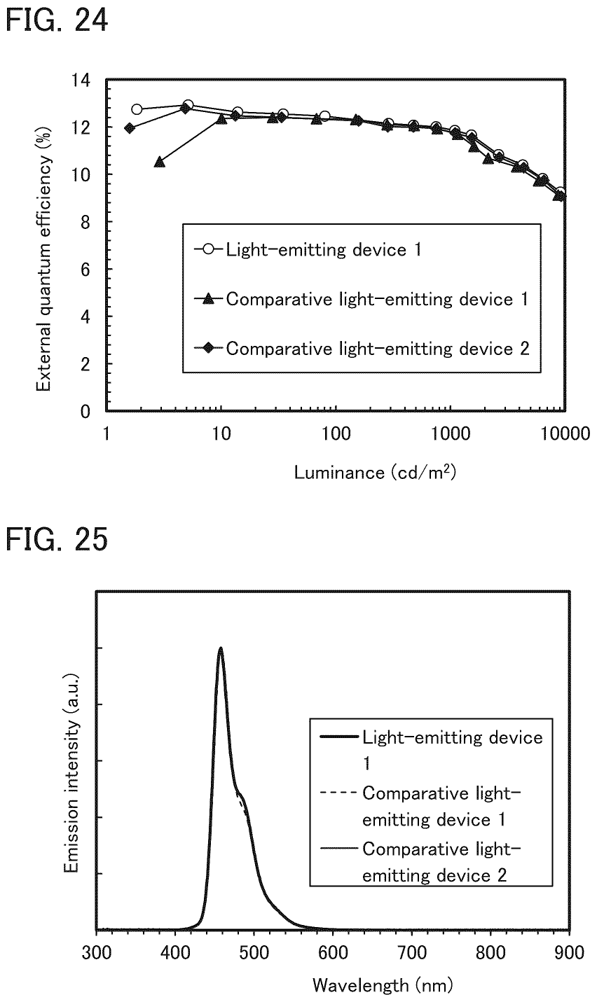

FIG. 20 shows the luminance-current density characteristics of the light-emitting device 1, the comparative light-emitting device 1, and the comparative light-emitting device 2; FIG. 21, the current efficiency-luminance characteristics; FIG. 22, the luminance-voltage characteristics; FIG. 23, the current-voltage characteristics; FIG. 24, the external quantum efficiency-luminance characteristics; and FIG. 25, the emission spectra. In addition, Table 2 shows the main characteristics of the light-emitting device 1, the comparative light-emitting device 1, and the comparative light-emitting device 2 at around 1000 cd/m².

TABLE 2

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.9 | 0.38 | 9.6 | 0.14 | 0.12 | 11.4 | 11.9 |
| device 1 | | | | | | | |
| Comparative light- | 3.8 | 0.41 | 10.3 | 0.14 | 0.12 | 11.2 | 11.7 |
| emitting device 1 | | | | | | | |
| Comparative light- | 3.9 | 0.38 | 9.6 | 0.14 | 0.12 | 11.5 | 11.7 |
| emitting device 2 | | | | | | | |

It was found from FIG. 20 to FIG. 25 and Table 2 that the light-emitting device 1, the comparative light-emitting device 1, and the comparative light-emitting device 2 are blue-light-emitting devices with favorable characteristics.

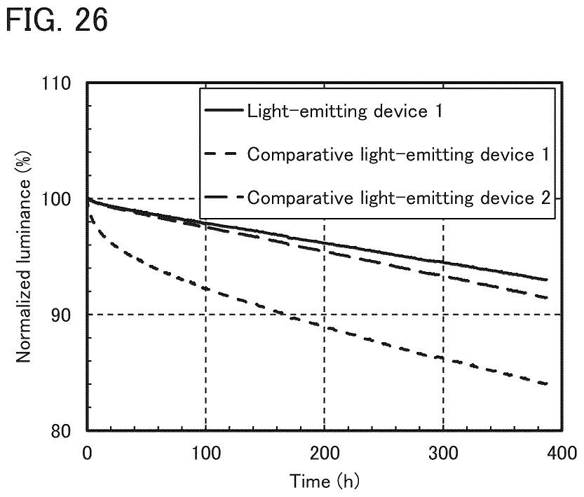

FIG. 26 is a graph showing a change in luminance over driving time at a current density of 50 mA/cm². The light-emitting device 1 of one embodiment of the present invention was found to be a light-emitting device exhibiting a longer lifetime than the comparative light-emitting device 1, which uses as the host material an anthracene compound in which a naphthyl group is bonded to the P-position, and the comparative light-emitting device 2, which uses as the host material an anthracene compound in which an a-naphthyl group is bonded to the 2-position and the 10-position of anthracene and a phenyl group is bonded to the 9-position.

### Example 4

Described in this example is the light-emitting device 2, which uses the anthracene compound for a host material of one embodiment of the present invention described in Embodiment 1. Also shown are the comparative light-emitting device 3 and the comparative light-emitting device 4, each of which uses as a host material an organic compound having a structure similar to that of the anthracene compound of one embodiment of the present invention. Structural formulae of organic compounds used for the light-emitting device 2, the comparative light-emitting device 3, and the comparative light-emitting device 4 are shown below.

### [Chemical Formula 16]

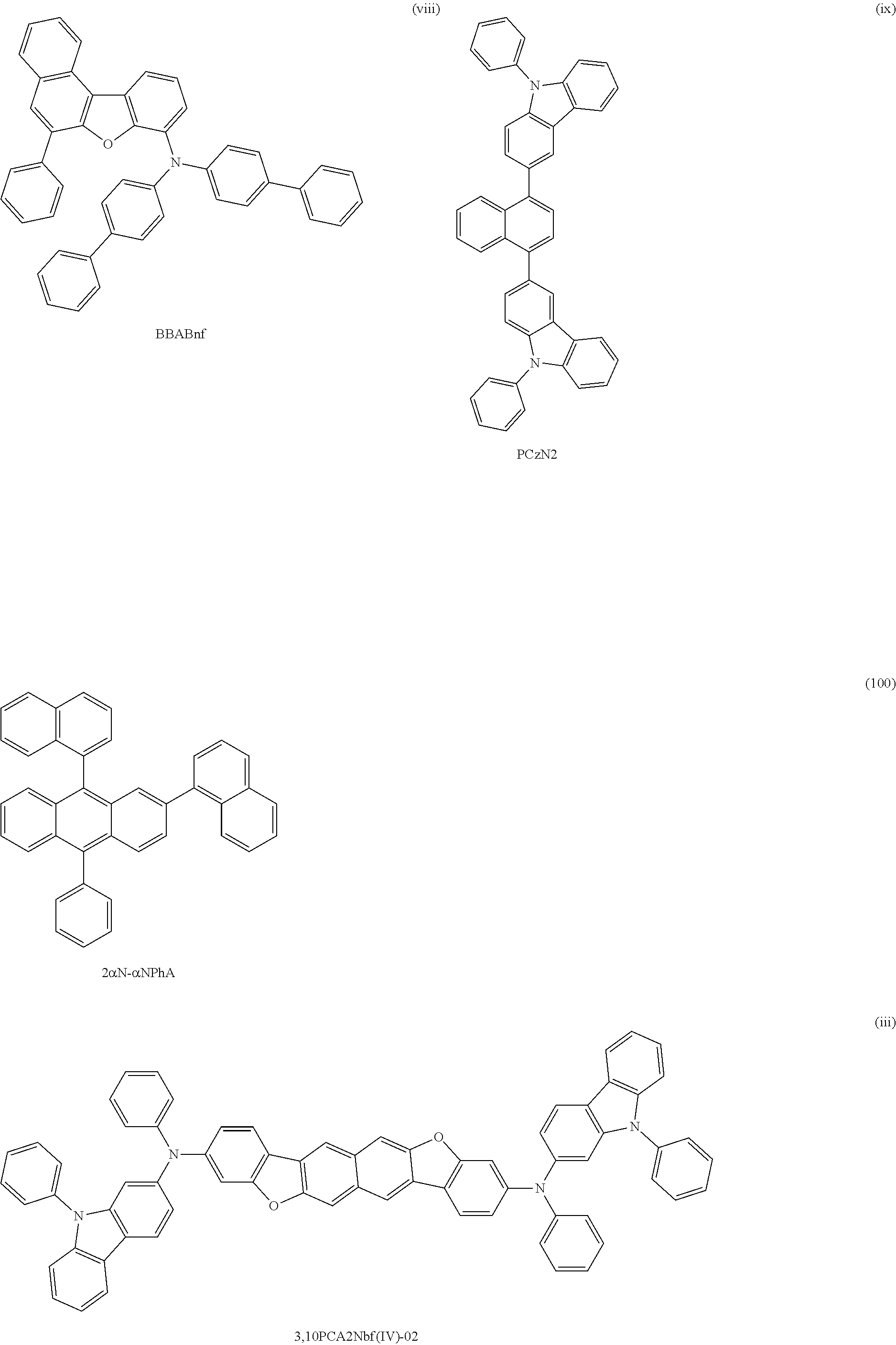

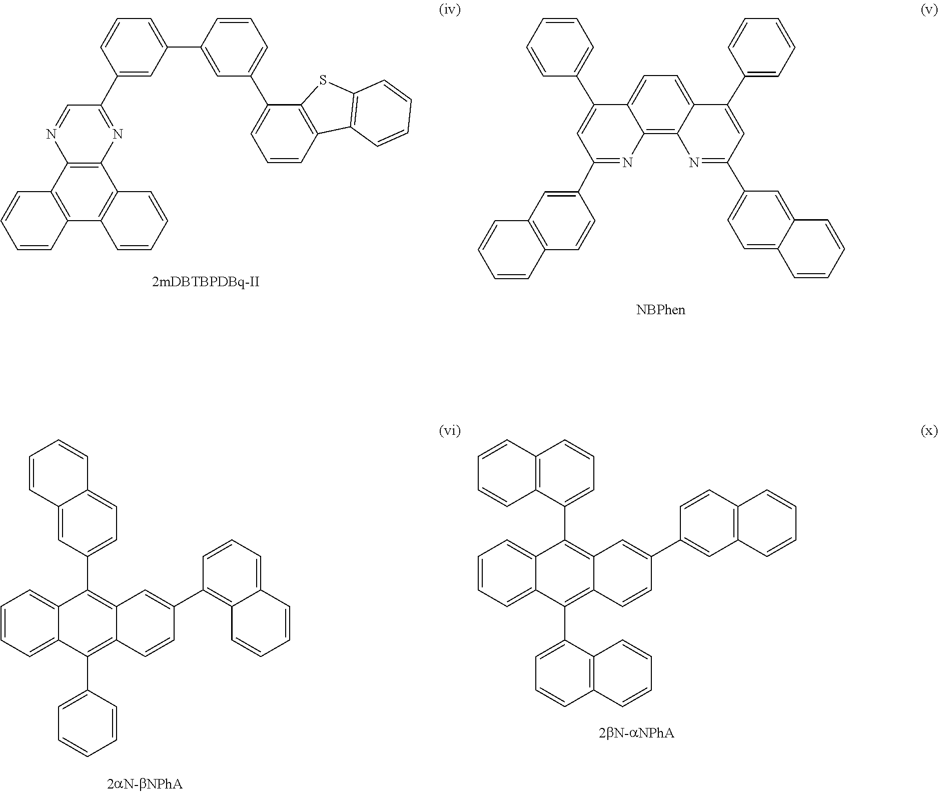

### (Fabrication Method of Light-Emitting Device 2)

First, a film of indium tin oxide containing silicon oxide (ITSO) was formed over a glass substrate by a sputtering method, so that the first electrode 101 was formed. Note that the film thickness was 70 nm and the area of the electrode was 2 mm×2 mm.

Next, in pretreatment for forming the light-emitting device over the substrate, the surface of the substrate was washed with water and baked at 200° C. for one hour, and then UV ozone treatment was performed for 370 seconds.

After that, the substrate was transferred into a vacuum evaporation apparatus in which the pressure was reduced to approximately 10⁻⁴ Pa, vacuum baking at 170° C. for 30 minutes was performed in a heating chamber in the vacuum evaporation apparatus, and then the substrate was cooled down for approximately 30 minutes.

Next, the substrate over which the first electrode 101 was formed was fixed to a substrate holder provided in the vacuum evaporation apparatus so that the surface over which the first electrode 101 was formed faced downward, and N,N-bis(4-biphenyl)-6-phenylbenzo[b]naphtho[1,2-d]furan-8-amine (abbreviation: BBABnf) represented by Structural Formula (viii) above and ALD-MP001Q (produced by Analysis Atelier Corporation, material serial No. 1S20170124) were co-evaporated over the first electrode 101 to have a weight ratio of 1:0.1 (=BBABnf: ALD-MP001Q) to a thickness of 10 nm by an evaporation method using resistance heating, whereby the hole-injection layer 111 was formed.

Next, as the first hole-transport layer 112-1, BBABnf was deposited by evaporation to a thickness of 20 nm over the hole-injection layer 111, and then, as the second hole-transport layer 112-2, 3,3′-(naphthalene-1,4-diyl)bis(9-phenyl-9H-carbazole) (abbreviation: PCzN2) represented by Structural Formula (ix) above was deposited by evaporation to a thickness of 10 nm, whereby the hole-transport layer 112 was formed. Note that the second hole-transport layer 112-2 also functions as an electron-blocking layer.

Subsequently, 2,9-di(1-naphthyl)-10-phenylanthracene (abbreviation: 2αN-αNPhA) represented by Structural Formula (100) above, and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iii) above were deposited by co-evaporation to a thickness of 25 nm at a weight ratio of 1:0.015 (=2αN-αNPhA: 3,10PCA2Nbf(IV)-02), so that the light-emitting layer 113 was formed.

After that, over the light-emitting layer 113, 2-[3′-(dibenzothiophen-4-yl)biphenyl-3-yl]dibenzo[f,h]quinoxaline (abbreviation: 2mDBTBPDBq-II) represented by Structural Formula (iv) above was deposited by evaporation to a thickness of 15 nm, and then, 2,9-di(2-naphthyl)-4,7-diphenyl-1,10-phenanthroline (abbreviation: NBPhen) represented by Structural Formula (v) above was deposited by evaporation to a thickness of 10 nm, so that the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 2 of this example was fabricated.

### (Fabrication Method of Comparative Light-Emitting Device 3)

The comparative light-emitting device 3 was fabricated in a manner similar to that for the light-emitting device 2 except that 2αN-αNPhA in the light-emitting device 2 was replaced with 2-(1-naphthyl)-9-(2-naphthyl)-10-phenylanthracene (abbreviation: 2αN-QNPhA) represented by Structural Formula (vii) above.

### (Fabrication Method of Comparative Light-Emitting Device 4)

The comparative light-emitting device 4 was fabricated in a manner similar to that for the light-emitting device 2 except that 2αN-αNPhA in the light-emitting device 2 was replaced with 9-(1-naphthyl)-2-(2-naphthyl)-10-phenylanthracene (abbreviation: 2βN-αNPhA) represented by Structural Formula (x) above.

The device structures of the light-emitting device 2, the comparative light-emitting device 3, and the comparative light-emitting device 4 are listed in the following table.

TABLE 3

| | Hole- | | | Light- | | | Electron- |
|---|---|---|---|---|---|---|---|
| | injection | Hole-transport layer | | emitting | | | injection |
| | layer | 1 | 2 | layer | Electron-transport layer | | layer |
| | 10 nm | 20 nm | 10 nm | 25 nm | 15 nm | 10 nm | 1 nm |
| Light- | BBABnf: | BBABnf | PCzN2 | *4 | 2mDBTBPDBq-II | NBPhen | LiF |
| emitting | ALD- | | | | | | |
| device 2 | MP001Q | | | | | | |
| Comparative | (1:0.1) | | | *5 | | | |
| light-emitting | | | | | | | |
| device 3 | | | | | | | |
| Comparative | | | | *6 | | | |
| light-emitting | | | | | | | |
| device 4 | | | | | | | |

*4 2αN-αNPhA:3,10PCA2Nbf(IV)-02(1:0.015)
*5 2αN-βNPhA:3,10PCA2Nbf(IV)-02(1:0.015)
*6 2βN-αNPhA:3,10PCA2Nbf(IV)-02(1:0.015)

These light-emitting devices were subjected to sealing with a glass substrate (a sealant was applied to surround the devices, followed by UV treatment and one-hour heat treatment at 80° C. at the time of sealing) in a glove box containing a nitrogen atmosphere so that the light-emitting devices are not exposed to the air. Then, the initial characteristics and reliability of these light-emitting devices were measured. Note that the measurement was performed at room temperature.

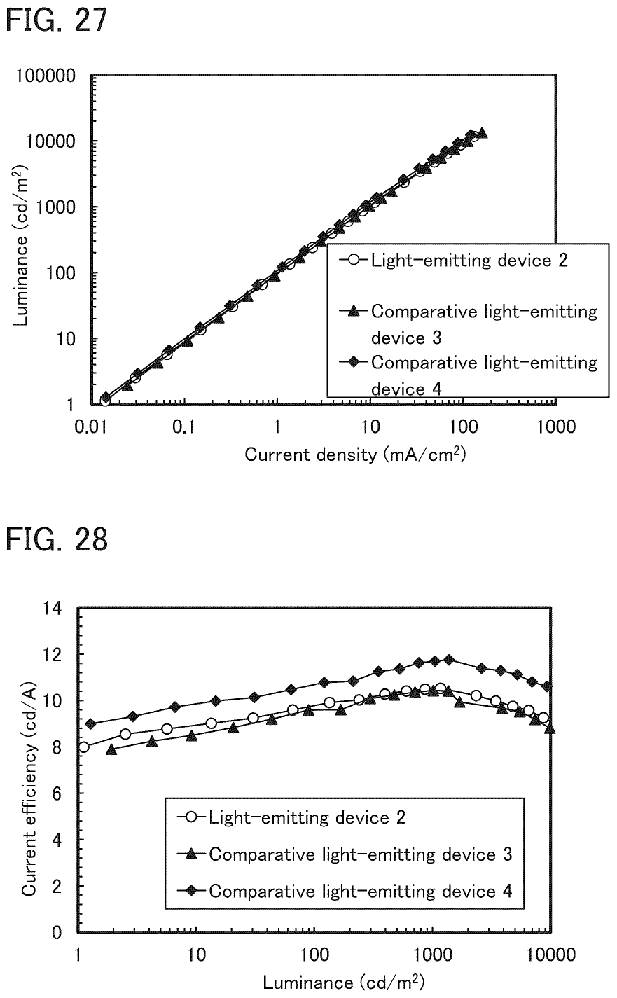

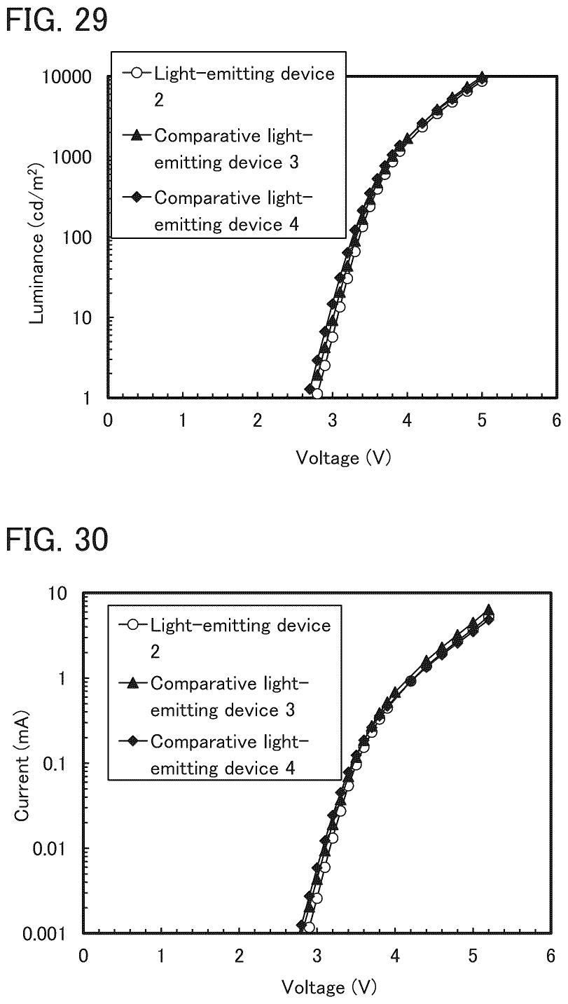

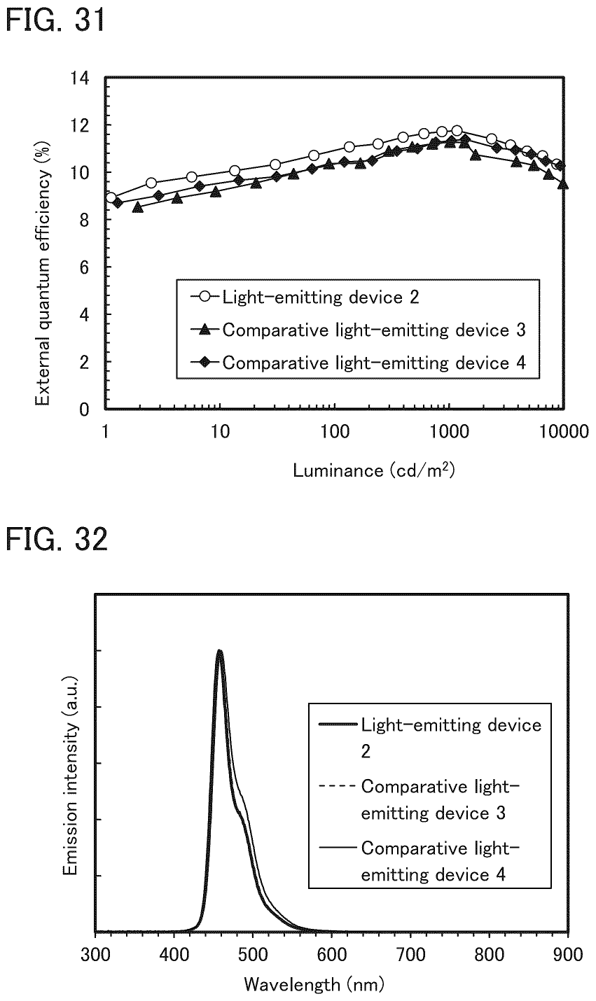

FIG. 27 shows the luminance-current density characteristics of the light-emitting device 2, the comparative light-emitting device 3, and the comparative light-emitting device 4; FIG. 28, the current efficiency-luminance characteristics; FIG. 29, the luminance-voltage characteristics; FIG. 30, the current-voltage characteristics; FIG. 31, the external quantum efficiency-luminance characteristics; and FIG. 32, the emission spectra. In addition, Table 4 shows the main characteristics of the light-emitting device 2, the comparative light-emitting device 3, and the comparative light-emitting device 4 at around 1000 cd/m².

TABLE 4

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 3.8 | 0.33 | 8.2 | 0.14 | 0.11 | 10.5 | 11.7 |
| device 2 | | | | | | | |
| Comparative light- | 3.8 | 0.39 | 9.8 | 0.14 | 0.11 | 10.4 | 11.3 |
| emitting device 3 | | | | | | | |
| Comparative light- | 3.8 | 0.36 | 9.0 | 0.14 | 0.13 | 11.7 | 11.3 |
| emitting device 4 | | | | | | | |

It was found from FIG. 27 to FIG. 32 and Table 4 that the light-emitting device 2 of one embodiment of the present invention, the comparative light-emitting device 3, and the comparative light-emitting device 4 are blue-light-emitting devices with favorable characteristics.

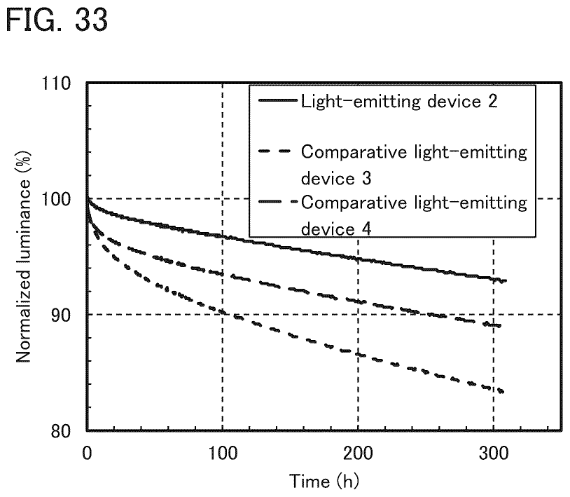

FIG. 33 is a graph showing a change in luminance over driving time at a current density of 50 mA/cm². The light-emitting device 2, which uses as the host material the anthracene compound for a host material of one embodiment of the present invention, exhibited more favorable characteristics than the comparative light-emitting device 3 and the comparative light-emitting device 4, each of which uses as the host material an anthracene compound in which a naphthyl group is bonded to the P-position.

### Example 5

Described in this example are the light-emitting device 3 and the light-emitting device 4, each of which uses the anthracene compound for a host material of one embodiment of the present invention described in Embodiment 1. Also shown are the comparative light-emitting device 5 to the comparative light-emitting device 10, each of which uses as a host material an organic compound having a structure similar to that of the anthracene compound of one embodiment of the present invention. Structural formulae of organic compounds used for the light-emitting device 3, the light-emitting device 4, and the comparative light-emitting device 5 to the comparative light-emitting device 10 are shown below.

### [Chemical Formula 17]

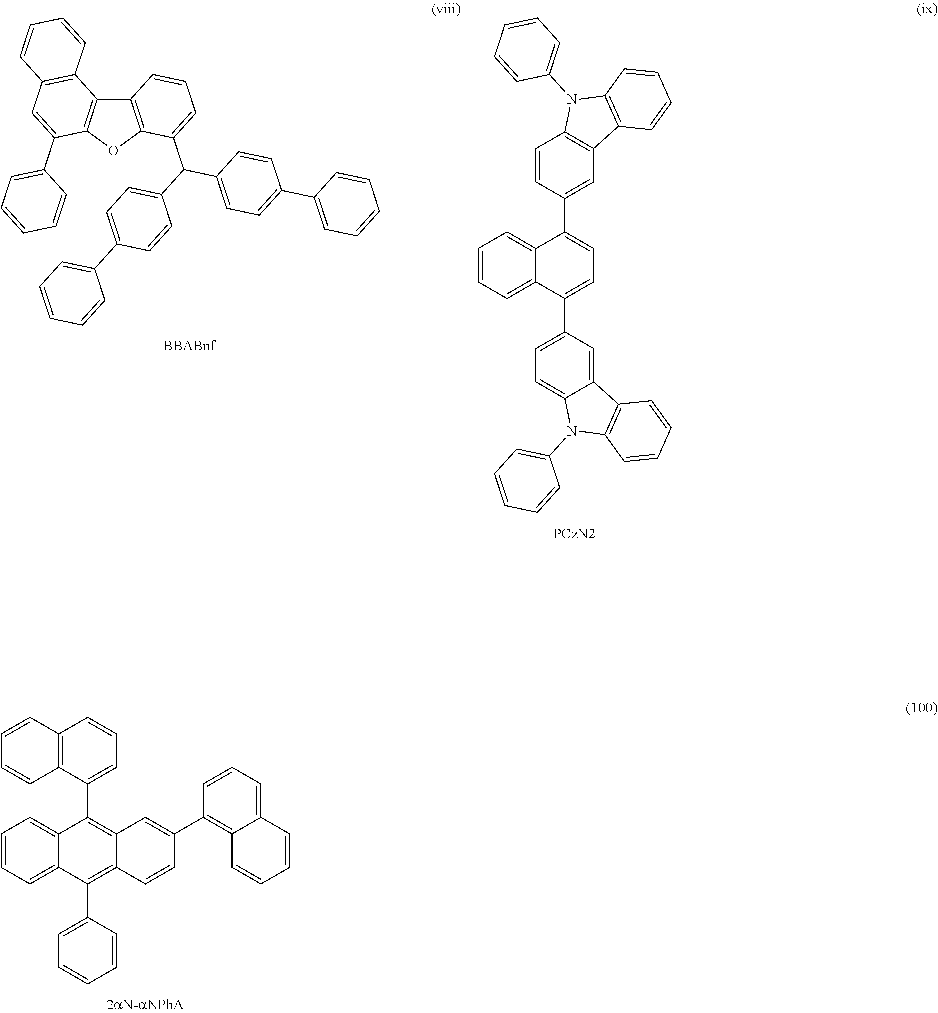

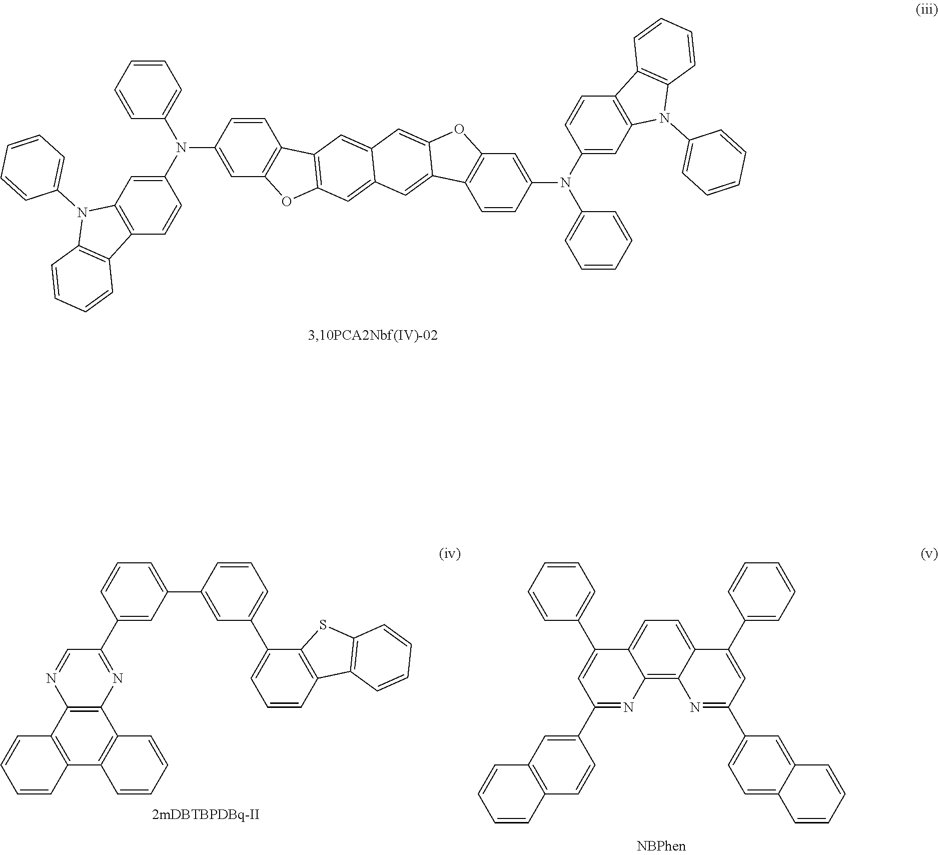 [SMILES: c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1 ; c1cc(-c2cccc(-c3cccc4c3sc3ccccc34)c2)cc(-c2cnc3c4ccccc4c4ccccc4c3n2)c1 ; c1ccc(-c2cc(-c3ccc4ccccc4c3)nc3c2ccc2c(-c4ccccc4)cc(-c4ccc5ccccc5c4)nc23)cc1]

### [Chemical Formula 18]

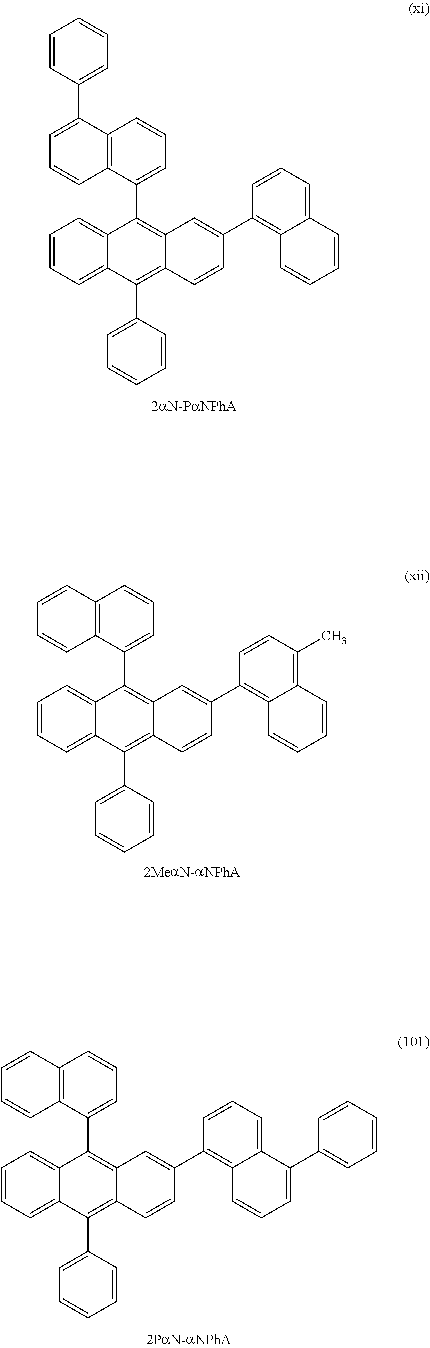

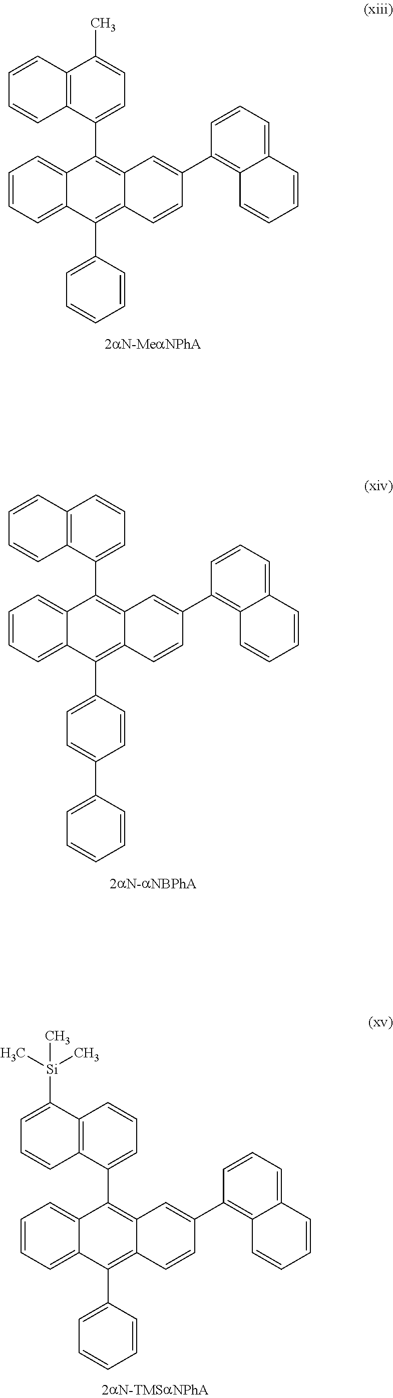

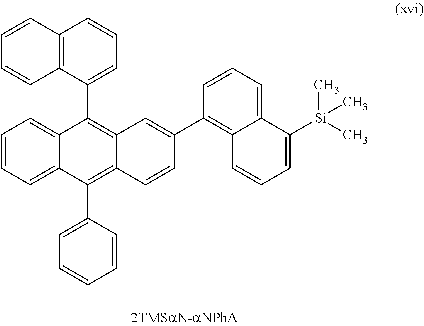 [SMILES: C[Si](C)(C)c1cccc2c(-c3ccc4c(-c5ccccc5)c5ccccc5c(-c5cccc6ccccc56)c4c3)cccc12]

### (Fabrication Method of Light-Emitting Device 3)

First, a film of indium tin oxide containing silicon oxide (ITSO) was formed over a glass substrate by a sputtering method, so that the first electrode 101 was formed. Note that the film thickness was 70 nm and the area of the electrode was 2 mm×2 mm.

Next, in pretreatment for forming the light-emitting device over the substrate, the surface of the substrate was washed with water and baked at 200° C. for one hour, and then UV ozone treatment was performed for 370 seconds.

After that, the substrate was transferred into a vacuum evaporation apparatus in which the pressure was reduced to approximately 10-4 Pa, vacuum baking at 170° C. for 30 minutes was performed in a heating chamber in the vacuum evaporation apparatus, and then the substrate was cooled down for approximately 30 minutes.

Next, the substrate over which the first electrode 101 was formed was fixed to a substrate holder provided in the vacuum evaporation apparatus so that the surface over which the first electrode 101 was formed faced downward, and N,N-bis(4-biphenyl)-6-phenylbenzo[b]naphtho[1,2-d]furan-8-amine (abbreviation: BBABnf) represented by Structural Formula (viii) above and ALD-MP001Q (produced by Analysis Atelier Corporation, material serial No. 1S20170124) were co-evaporated over the first electrode 101 to have a weight ratio of 1:0.1 (=BBABnf: ALD-MP001Q) to a thickness of 10 nm by an evaporation method using resistance heating, whereby the hole-injection layer 111 was formed.

Next, as the first hole-transport layer 112-1, BBABnf was deposited by evaporation to a thickness of 20 nm over the hole-injection layer 111, and then, as the second hole-transport layer 112-2, 3,3′-(naphthalene-1,4-diyl)bis(9-phenyl-9H-carbazole) (abbreviation: PCzN2) represented by Structural Formula (ix) above was deposited by evaporation to a thickness of 10 nm, whereby the hole-transport layer 112 was formed. Note that the second hole-transport layer 112-2 also functions as an electron-blocking layer.

Subsequently, 2,9-di(1-naphthyl)-10-phenylanthracene (abbreviation: 2αN-αNPhA) represented by Structural Formula (100) above, and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iii) above were deposited by co-evaporation to a thickness of 25 nm at a weight ratio of 1:0.015 (=2αN-αNPhA: 3,10PCA2Nbf(IV)-02), so that the light-emitting layer 113 was formed.

After that, over the light-emitting layer 113, 2-[3′-(dibenzothiophen-4-yl)biphenyl-3-yl]dibenzo[f,h]quinoxaline (abbreviation: 2mDBTBPDBq-II) represented by Structural Formula (iv) above was deposited by evaporation to a thickness of 15 nm, and then, 2,9-di(2-naphthyl)-4,7-diphenyl-1,10-phenanthroline (abbreviation: NBPhen) represented by Structural Formula (v) above was deposited by evaporation to a thickness of 10 nm, so that the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 3 of this example was fabricated.

### (Fabrication Method of Light-Emitting Device 4)

The light-emitting device 4 was fabricated in a manner similar to that for the light-emitting device 3 except that 2αN-αNPhA in the light-emitting device 3 was replaced with 9-(1-naphthyl)-10-phenyl-2-(5-phenyl-1-naphthyl)anthracene (abbreviation: 2PαN-αNPhA) represented by Structural Formula (101) above.

### (Fabrication Method of Comparative Light-Emitting Device 5)

The comparative light-emitting device 5 was fabricated in a manner similar to that for the light-emitting device 3 except that 2αN-αNPhA in the light-emitting device 3 was replaced with 2-(1-naphthyl)-10-phenyl-9-(5-phenyl-1-naphthyl)anthracene (abbreviation: 2αN-PαNPhA) represented by Structural Formula (xi) above.

### (Fabrication Method of Comparative Light-Emitting Device 6)

The comparative light-emitting device 6 was fabricated in a manner similar to that for the light-emitting device 3 except that 2αN-αNPhA in the light-emitting device 3 was replaced with 2-(4-methyl-1-naphthyl)-9-(1-naphthyl)-10-phenylanthracene (abbreviation: 2MeαN-αNPhA) represented by Structural Formula (xii) above.

### (Fabrication Method of Comparative Light-Emitting Device 7)

The comparative light-emitting device 7 was fabricated in a manner similar to that for the light-emitting device 3 except that 2αN-αNPhA in the light-emitting device 3 was replaced with 9-(4-methyl-1-naphthyl)-2-(1-naphthyl)-10-phenylanthracene (abbreviation: 2αN-MeαNPhA) represented by Structural Formula (xiii) above.

### (Fabrication Method of Comparative Light-Emitting Device 8)

The comparative light-emitting device 8 was fabricated in a manner similar to that for the light-emitting device 3 except that 2αN-αNPhA in the light-emitting device 3 was replaced with 10-(4-biphenyl)-2,9-di(1-naphthyl)anthracene (abbreviation: 2αN-αNBPhA) represented by Structural Formula (xiv) above.

### (Fabrication Method of Comparative Light-Emitting Device 9)

The comparative light-emitting device 9 was fabricated in a manner similar to that for the light-emitting device 3 except that 2αN-αNPhA in the light-emitting device 3 was replaced with 2-(1-naphthyl)-10-phenyl-9-(5-trimethylsilyl-1-naphthyl)anthracene (abbreviation: 2αN-TMSαNPhA) represented by Structural Formula (xv) above.

### (Fabrication Method of Comparative Light-Emitting Device 10)

The comparative light-emitting device 10 was fabricated in a manner similar to that for the light-emitting device 3 except that 2αN-αNPhA in the light-emitting device 3 was replaced with 9-(1-naphthyl)-10-phenyl-2-(5-trimethylsilyl-1-naphthyl)anthracene (abbreviation: 2TMSαN-αNPhA) represented by Structural Formula (xvi) above.

The device structures of the light-emitting device 3, the light-emitting device 4, and the comparative light-emitting device 5 to the comparative light-emitting device 10 are listed in the following table.

TABLE 5

| | Hole- | | | Light- | | | Electron- |
|---|---|---|---|---|---|---|---|
| | injection | Hole-transport layer | | emitting | | | injection |
| | layer | 1 | 2 | layer | Electron-transport layer | | layer |
| | 10 nm | 20 nm | 10 nm | 25 nm | 15 nm | 10 nm | 1 nm |
| Light-emitting | BBABnf: | BBABnf | PCzN2 | *7 | 2mDBTBPDBq-II | NBPhen | LiF |
| device 3 | ALD- | | | | | | |
| Light-emitting | MP001Q | | | *8 | | | |
| device 4 | (1:0.1) | | | | | | |
| Comparative | | | | *9 | | | |
| light-emitting | | | | | | | |
| device 5 | | | | | | | |
| Comparative | | | | *10 | | | |
| light-emitting | | | | | | | |
| device 6 | | | | | | | |
| Comparative | | | | *11 | | | |
| light-emitting | | | | | | | |
| device 7 | | | | | | | |
| Comparative | | | | *12 | | | |
| light-emitting | | | | | | | |
| device 8 | | | | | | | |
| Comparative | | | | *13 | | | |
| light-emitting | | | | | | | |
| device 9 | | | | | | | |
| Comparative | | | | *14 | | | |
| light-emitting | | | | | | | |
| device 10 | | | | | | | |

*7 2αN-αNPhA:3,10PCA2Nbf(IV)-02(1:0.015)
*8 2PαN-αNPhA:3,10PCA2Nbf(IV)-02(1:0.015)
*9 2αN-PαNPhA:3,10PCA2Nbf(IV)-02(1:0.015)
*10 2MeαN-αNPhA:3,10PCA2Nbf(IV)-02(1:0.015)
*11 2αN-MeaNPhA:3,10PCA2Nbf(IV)-02(1:0.015)
*12 2αN-αNBPhA:3,10PCA2Nbf(IV)-02(1:0.015)
*13 2αN-TMSαNPhA:3,10PCA2Nbf(IV)-02(1:0.015)
*14 2TMSαN-αNPhA:3,10PCA2Nbf(IV)-02(1:0.015)

These light-emitting devices were subjected to sealing with a glass substrate (a sealant was applied to surround the devices, followed by UV treatment and one-hour heat treatment at 80° C. at the time of sealing) in a glove box containing a nitrogen atmosphere so that the light-emitting devices are not exposed to the air. Then, the initial characteristics and reliability of these light-emitting devices were measured. Note that the measurement was performed at room temperature.

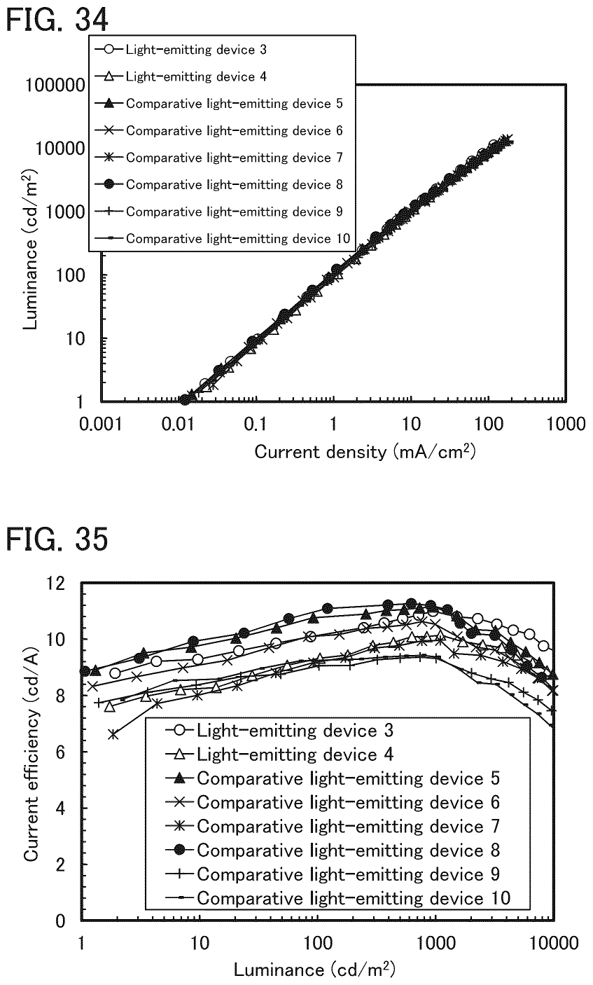

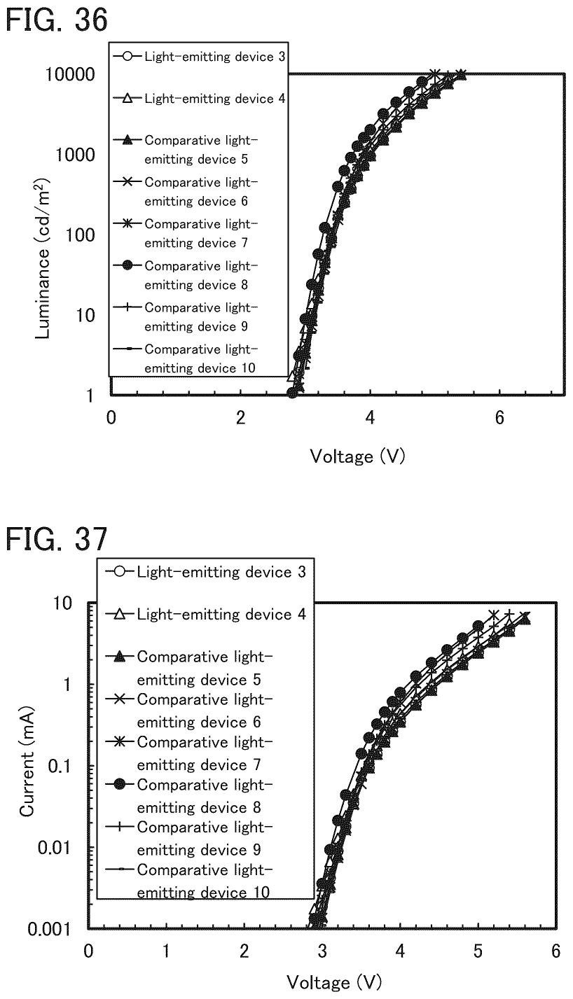

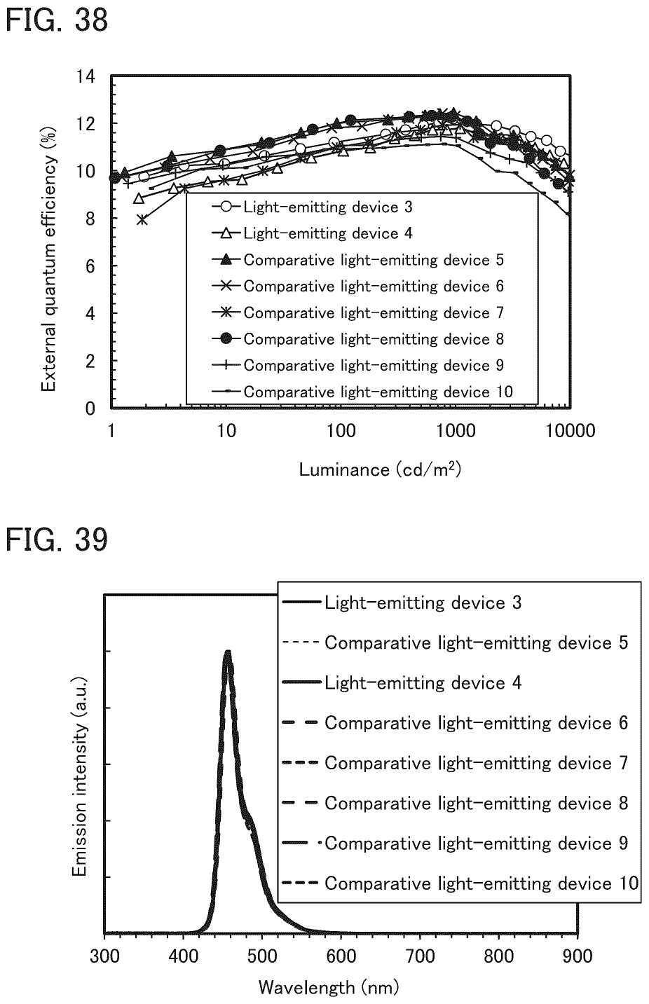

FIG. 34 shows the luminance-current density characteristics of the light-emitting device 3, the light-emitting device 4, and the comparative light-emitting device 5 to the comparative light-emitting device 10; FIG. 35, the current efficiency-luminance characteristics; FIG. 36, the luminance-voltage characteristics; FIG. 37, the current-voltage characteristics; FIG. 38, the external quantum efficiency-luminance characteristics; and FIG. 39, the emission spectra. In addition, Table 6 shows the main characteristics of the light-emitting device 3, the light-emitting device 4, and the comparative light-emitting device 5 to the comparative light-emitting device 10 at around 1000 cd/m².

TABLE 6

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting | 4.0 | 0.35 | 8.7 | 0.14 | 0.11 | 11.0 | 12.2 |
| device 3 | | | | | | | |
| Light-emitting | 4.0 | 0.38 | 9.4 | 0.14 | 0.10 | 10.5 | 12.3 |
| device 4 | | | | | | | |
| Comparative light- | 4.0 | 0.35 | 8.6 | 0.14 | 0.11 | 11.1 | 12.4 |
| emitting device 5 | | | | | | | |
| Comparative light- | 4.0 | 0.43 | 10.8 | 0.14 | 0.10 | 10.1 | 11.8 |
| emitting device 6 | | | | | | | |
| Comparative light- | 3.9 | 0.44 | 11.0 | 0.14 | 0.09 | 10.0 | 11.9 |
| emitting device 7 | | | | | | | |
| Comparative light- | 3.7 | 0.32 | 8.1 | 0.14 | 0.11 | 11.2 | 12.2 |
| emitting device 8 | | | | | | | |
| Comparative light- | 3.9 | 0.43 | 10.8 | 0.14 | 0.09 | 9.3 | 11.4 |
| emitting device 9 | | | | | | | |
| Comparative light- | 4.0 | 0.44 | 10.9 | 0.14 | 0.10 | 9.4 | 11.1 |
| emitting device 10 | | | | | | | |

It was found from FIG. 34 to FIG. 39 and Table 6 that the light-emitting device 3, the light-emitting device 4, and the comparative light-emitting device 5 to the comparative light-emitting device 10 are blue-light-emitting devices with favorable characteristics.

The LT97 (the time until when the luminance decreases to 97% of the initial luminance) and the LT95 (the time until when the luminance decreases to 95% of the initial luminance) of each light-emitting device at a current density of 50 mA/cm² are listed in the following table.

TABLE 7

| | LT97 | LT95 |
|---|---|---|
| | (Time) | (Time) |
| Light-emitting device 3 | 98 | 230 |
| Light-emitting device 4 | 46 | 99 |
| Comparative light-emitting device 5 | 34 | 97 |
| Comparative light-emitting device 6 | 13 | 39 |
| Comparative light-emitting device 7 | 38 | 92 |
| Comparative light-emitting device 8 | 35 | 103 |
| Comparative light-emitting device 9 | 10 | 20 |
| Comparative light-emitting device 10 | 7 | 15 |

The table indicates that the light-emitting devices using as the host material the anthracene compound for a host material of one embodiment of the present invention exhibited favorable characteristics.

It was found from the light-emitting device 3, the comparative light-emitting device 6, the comparative light-emitting device 7, the comparative light-emitting device 9, and the comparative light-emitting device 10 that the alkyl group and the alkylsilyl group bonded to the anthracene compound for a host material of one embodiment of the present invention affected the reliability. In particular, the effect of the alkylsilyl group is not negligible; it is found that the methyl group also has a relatively large effect regardless of its small size.

### Example 6

Described in this example is the light-emitting device 5, which uses the anthracene compound for a host material of one embodiment of the present invention described in Embodiment 1. Also shown are the comparative light-emitting device 11 and the comparative light-emitting device 12, each of which uses as a host material an organic compound having a structure similar to that of the anthracene compound of one embodiment of the present invention. Structural formulae of organic compounds used for the light-emitting device 5, the comparative light-emitting device 11, and the comparative light-emitting device 12 are shown below.

### [Chemical Formula 19]

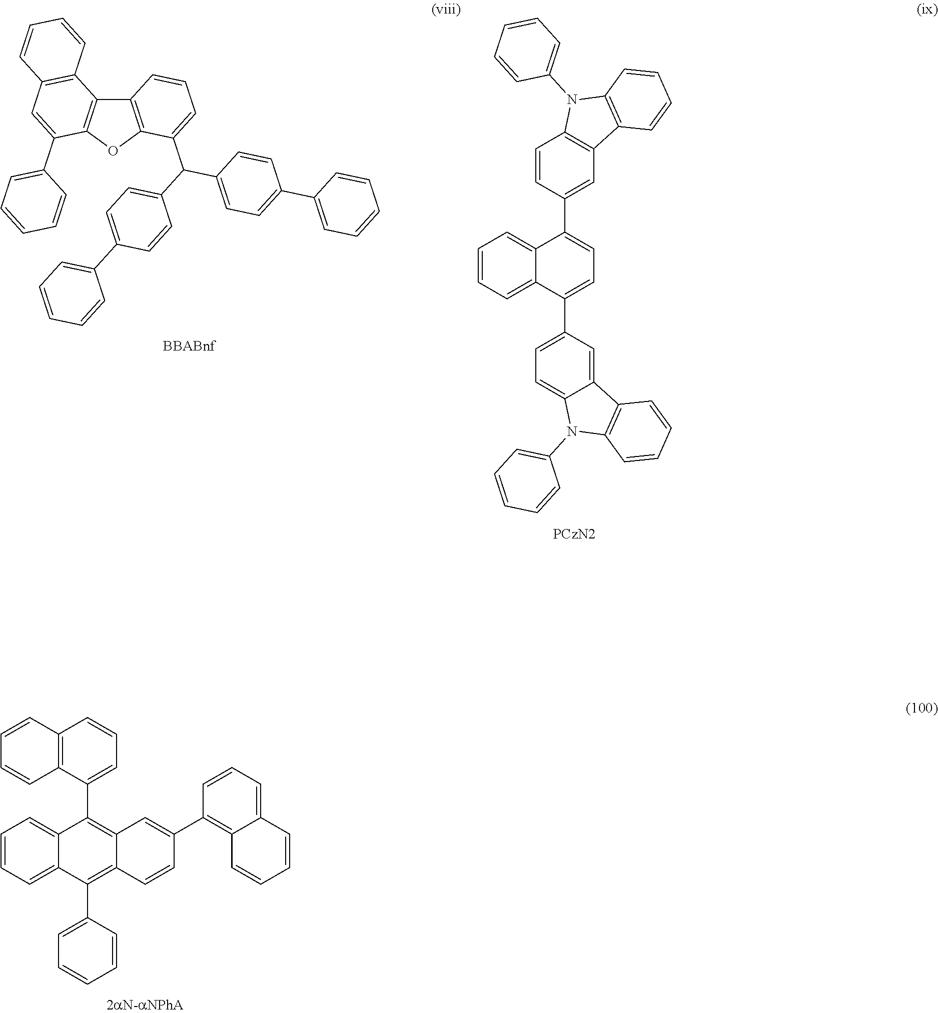

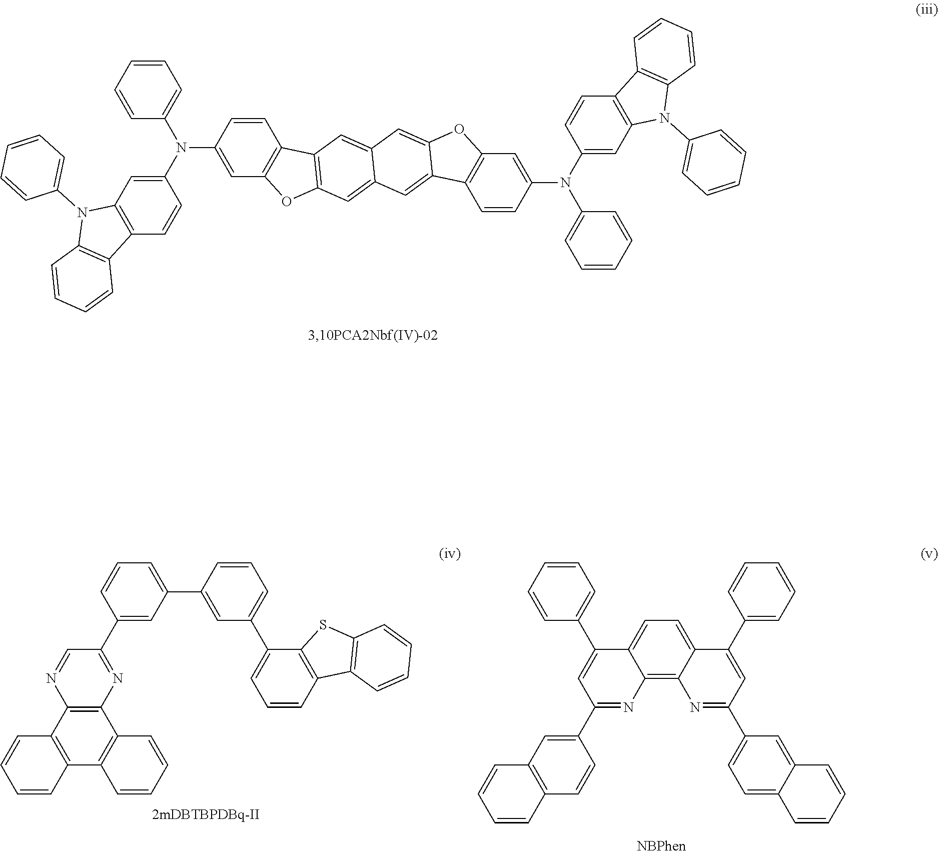 [SMILES: c1ccc(N(c2ccc3c(c2)oc2cc4cc5c(cc4cc23)oc2cc(N(c3ccccc3)c3ccc4c6ccccc6n(-c6ccccc6)c4c3)ccc25)c2ccc3c4ccccc4n(-c4ccccc4)c3c2)cc1 ; c1cc(-c2cccc(-c3cccc4c3sc3ccccc34)c2)cc(-c2cnc3c4ccccc4c4ccccc4c3n2)c1 ; c1ccc(-c2cc(-c3ccc4ccccc4c3)nc3c2ccc2c(-c4ccccc4)cc(-c4ccc5ccccc5c4)nc23)cc1]

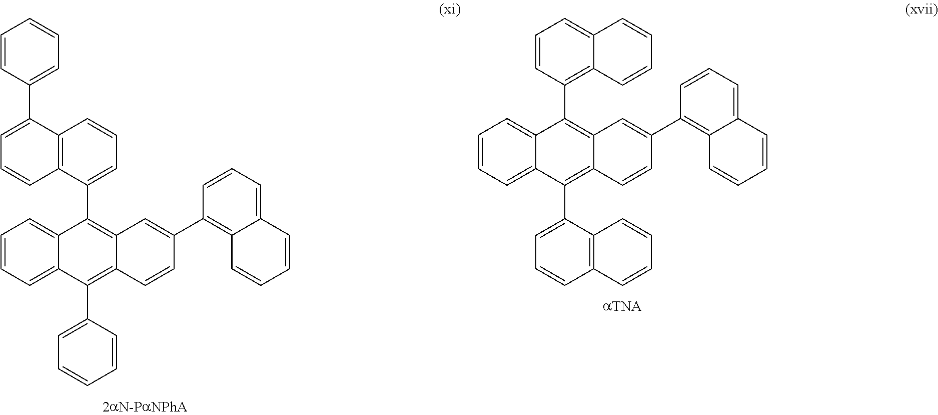

### (Fabrication Method of Light-Emitting Device 5)

First, a film of indium tin oxide containing silicon oxide (ITSO) was formed over a glass substrate by a sputtering method, so that the first electrode 101 was formed. Note that the film thickness was 70 nm and the area of the electrode was 2 mm×2 mm.

Next, in pretreatment for forming the light-emitting device over the substrate, the surface of the substrate was washed with water and baked at 200° C. for one hour, and then UV ozone treatment was performed for 370 seconds.

After that, the substrate was transferred into a vacuum evaporation apparatus in which the pressure was reduced to approximately 10⁴ Pa, vacuum baking at 170° C. for 30 minutes was performed in a heating chamber in the vacuum evaporation apparatus, and then the substrate was cooled down for approximately 30 minutes.

Next, the substrate over which the first electrode 101 was formed was fixed to a substrate holder provided in the vacuum evaporation apparatus so that the surface over which the first electrode 101 was formed faced downward, and N,N-bis(4-biphenyl)-6-phenylbenzo[b]naphtho[1,2-d]furan-8-amine (abbreviation: BBABnf) represented by Structural Formula (viii) above and ALD-MP001Q (produced by Analysis Atelier Corporation, material serial No. 1S20170124) were co-evaporated over the first electrode 101 to have a weight ratio of 1:0.1 (=BBABnf: ALD-MP001Q) to a thickness of 10 nm by an evaporation method using resistance heating, whereby the hole-injection layer 111 was formed.

Next, as the first hole-transport layer 112-1, BBABnf was deposited by evaporation to a thickness of 20 nm over the hole-injection layer 111, and then, as the second hole-transport layer 112-2, 3,3′-(naphthalene-1,4-diyl)bis(9-phenyl-9H-carbazole) (abbreviation: PCzN2) represented by Structural Formula (ix) above was deposited by evaporation to a thickness of 10 nm, whereby the hole-transport layer 112 was formed. Note that the second hole-transport layer 112-2 also functions as an electron-blocking layer.

Subsequently, 2,9-di(1-naphthyl)-10-phenylanthracene (abbreviation: 2αN-αNPhA) represented by Structural Formula (100) above, and 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02) represented by Structural Formula (iii) above were deposited by co-evaporation to a thickness of 25 nm at a weight ratio of 1:0.015 (=2αN-αNPhA: 3,10PCA2Nbf(IV)-02), so that the light-emitting layer 113 was formed.

After that, over the light-emitting layer 113, 2-[3′-(dibenzothiophen-4-yl)biphenyl-3-yl]dibenzo[f,h]quinoxaline (abbreviation: 2mDBTBPDBq-II) represented by Structural Formula (iv) above was deposited by evaporation to a thickness of 15 nm, and then, 2,9-di(2-naphthyl)-4,7-diphenyl-1,10-phenanthroline (abbreviation: NBPhen) represented by Structural Formula (v) above was deposited by evaporation to a thickness of 10 nm, so that the electron-transport layer 114 was formed.

After the formation of the electron-transport layer 114, lithium fluoride (LiF) was deposited by evaporation to a thickness of 1 nm to form the electron-injection layer 115, and then aluminum was deposited by evaporation to a thickness of 200 nm to form the second electrode 102, whereby the light-emitting device 5 of this example was fabricated.

### (Fabrication Method of Comparative Light-Emitting Device 11)

The comparative light-emitting device 11 was fabricated in a manner similar to that for the light-emitting device 5 except that 2αN-αNPhA in the light-emitting device 5 was replaced with 2-(1-naphthyl)-10-phenyl-9-(5-phenyl-1-naphthyl)anthracene (abbreviation: 2αN-PαNPhA) represented by Structural Formula (xi) above.

### (Fabrication Method of Comparative Light-Emitting Device 12)

The comparative light-emitting device 12 was fabricated in a manner similar to that for the light-emitting device 5 except that 2αN-αNPhA in the light-emitting device 5 was replaced with 2,9,10-tri(1-naphthyl)anthracene (abbreviation: αTNA) represented by Structural Formula (xvii) above.

The device structures of the light-emitting device 5, the comparative light-emitting device 11, and the comparative light-emitting device 12 are listed in the following table.

TABLE 8

| | Hole- | | | Light- | | | Electron- |
|---|---|---|---|---|---|---|---|
| | injection | Hole-transport layer | | emitting | | | injection |
| | layer | 1 | 2 | layer | Electron-transport layer | | layer |
| | 10 nm | 20 nm | 10 nm | 25 nm | 15 nm | 10 nm | 1 nm |
| Light-emitting | BBABnf: | BBABnf | PCZN2 | *15 | 2mDBTBPDBq-II | NBPhen | LiF |
| device 5 | ALD- | | | | | | |
| Comparative | MP001Q | | | *16 | | | |
| light-emitting | (1:0.1) | | | | | | |
| device 11 | | | | | | | |
| Comparative | | | | *17 | | | |
| light-emitting | | | | | | | |
| device 12 | | | | | | | |

*15 2αN-αNPhA:3,10PCA2Nbf(IV)-02(1:0.015)
*16 2αN-PαNPhA:3,10PCA2Nbf(IV)-02(1:0.015)
*17 αTNA:3,10PCA2Nbf(IV)-02(1:0.015)

These light-emitting devices were subjected to sealing with a glass substrate (a sealant was applied to surround the devices, followed by UV treatment and one-hour heat treatment at 80° C. at the time of sealing) in a glove box containing a nitrogen atmosphere so that the light-emitting devices are not exposed to the air. Then, the initial characteristics and reliability of these light-emitting devices were measured. Note that the measurement was performed at room temperature.

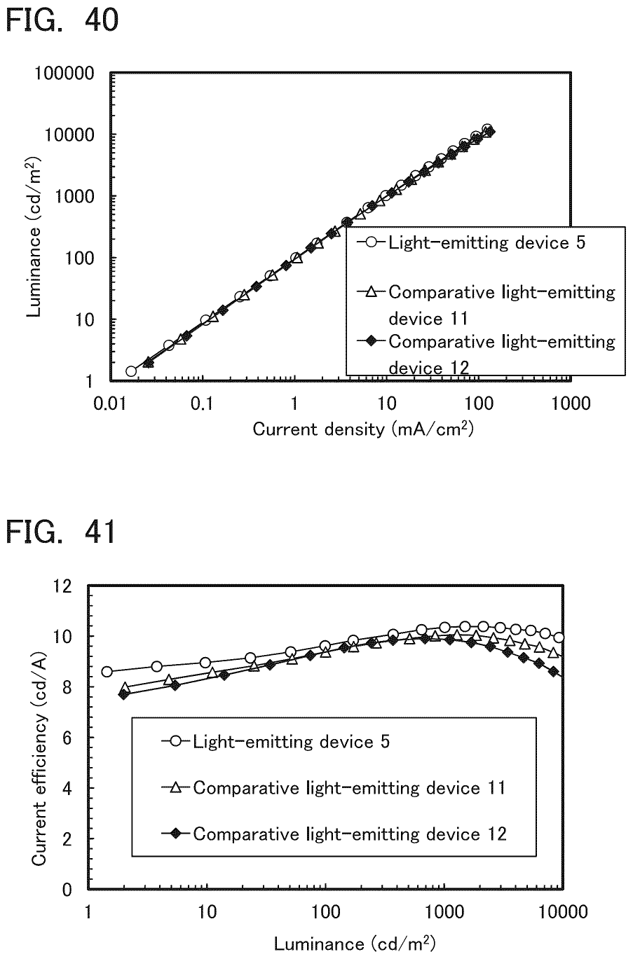

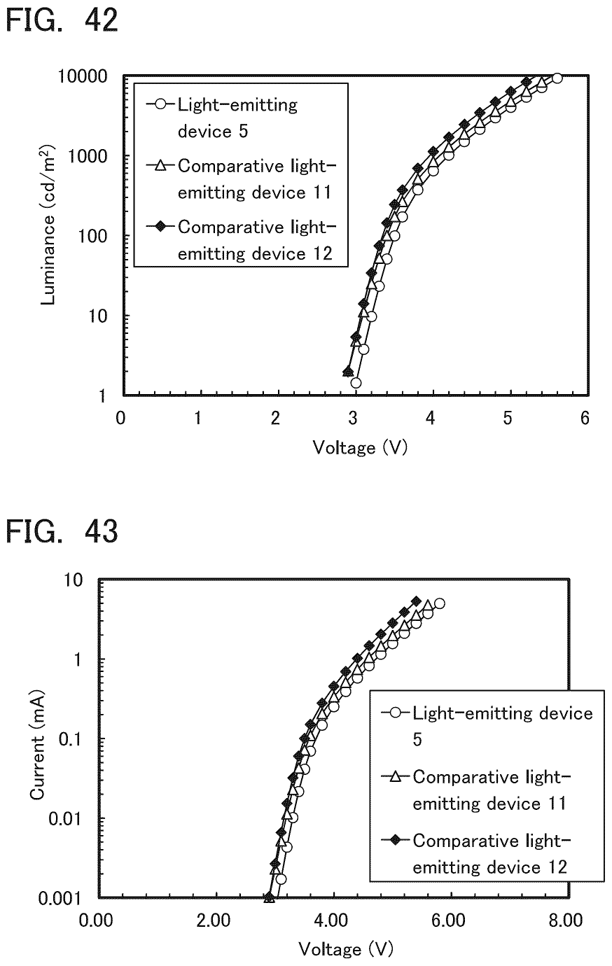

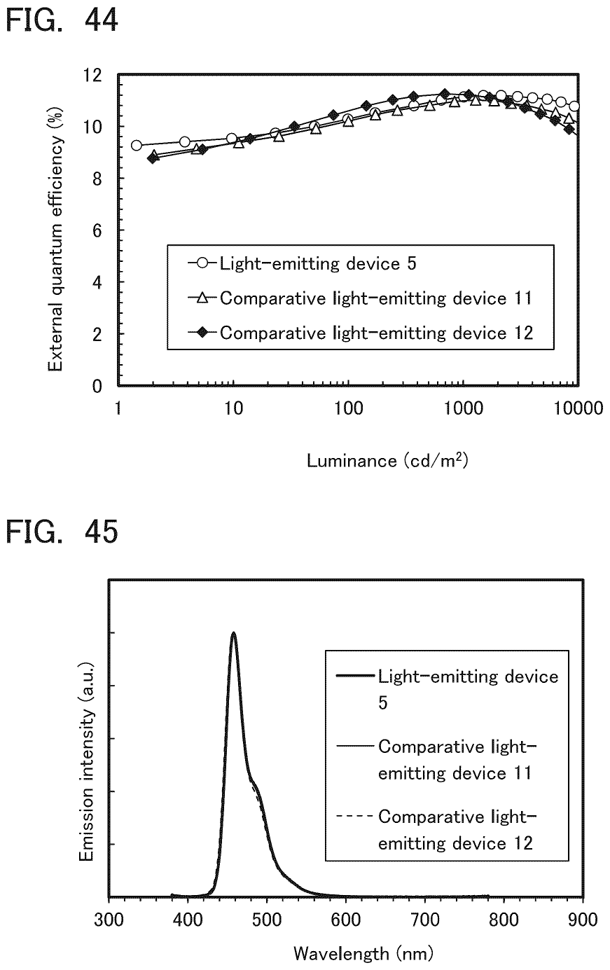

FIG. 40 shows the luminance-current density characteristics of the light-emitting device 5, the comparative light-emitting device 11, and the comparative light-emitting device 12; FIG. 41, the current efficiency-luminance characteristics; FIG. 42, the luminance-voltage characteristics; FIG. 43, the current-voltage characteristics; FIG. 44, the external quantum efficiency-luminance characteristics; and FIG. 45, the emission spectra. In addition, Table 9 shows the main characteristics of the light-emitting device 5, the comparative light-emitting device 11, and the comparative light-emitting device 12 at around 1000 cd/in.

TABLE 9

| | | | | | | | External |
|---|---|---|---|---|---|---|---|
| | | | Current | | | Current | quantum |
| | Voltage | Current | density | Chromaticity | Chromaticity | efficiency | efficiency |
| | (V) | (mA) | (mA/cm²) | x | y | (cd/A) | (%) |
| Light-emitting device 5 | 4.0 | 0.34 | 8.4 | 0.14 | 0.10 | 10.0 | 11.0 |
| Comparative light- | 4.0 | 0.35 | 8.8 | 0.14 | 0.10 | 10.1 | 11.5 |
| emitting device 11 | | | | | | | |
| Comparative light- | 4.0 | 0.31 | 7.7 | 0.14 | 0.11 | 10.7 | 11.3 |
| emitting device 12 | | | | | | | |

It was found from FIG. 40 to FIG. 45 and Table 9 that the light-emitting device 5 of one embodiment of the present invention, the comparative light-emitting device 11, and the comparative light-emitting device 12 are blue-light-emitting devices with favorable characteristics.

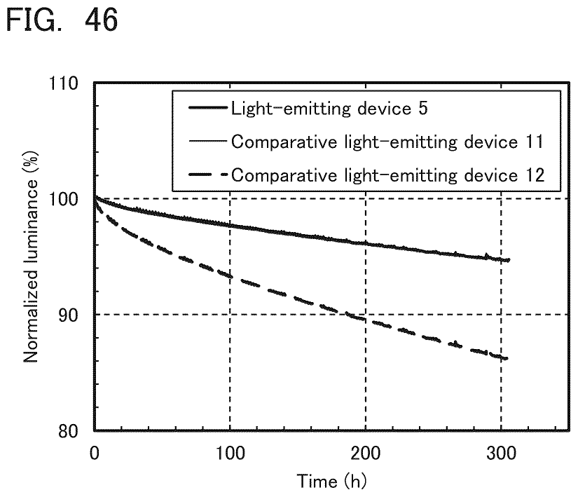

FIG. 46 is a graph showing a change in luminance over driving time at a current density of 50 mA/cm². The light-emitting device 5, which uses as the host material the anthracene compound for a host material of one embodiment of the present invention, exhibited more favorable characteristics than the comparative light-emitting device 11, which uses as the host material an anthracene compound in which a naphthyl group bonded to a phenyl group is bonded to the 9-position, and the comparative light-emitting device 12, which uses as the host material an anthracene compound to which three naphthyl groups are bonded.

### Reference Example 1

In this reference example, a synthesis method of 2-(4-methyl-1-naphthyl)-9-(1-naphthyl)-10-phenylanthracene (abbreviation: 2MeαN-αNPhA), which is the organic compound used as a comparative example in Example, will be described in detail. The structural formula of 2MeαN-αNPhA is shown below.

### [Chemical Formula 20]

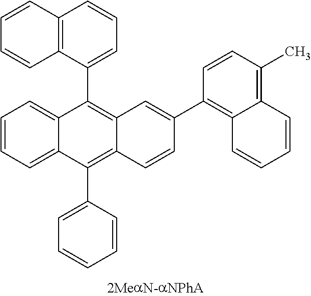 [SMILES: Cc1ccc(-c2ccc3c(-c4ccccc4)c4ccccc4c(-c4cccc5ccccc45)c3c2)c2ccccc12]

Into a 200 mL three-necked flask were put 1.4 g (3.4 mmol) of 2-chloro-9-(1-naphthyl)-10-phenylanthracene, 0.77 g (4.1 mmol) of 4-methyl-1-naphthylboronic acid, 0.13 g (0.36 mmol) of di(1-adamanthyl)-n-butylphosphine, 2.2 g (10 mmol) of tripotassium phosphate, 0.79 g (11 mmol) of tert-butyl alcohol, and 17 mL of diethylene glycol dimethyl ether, and the mixture was degassed by being stirred under reduced pressure. To the mixture was added 41 mg (0.18 mmol) of palladium(II) acetate, and the mixture was stirred at 130° C. under a nitrogen stream for 6 hours. After the stirring, water was added to the obtained mixture, and an aqueous layer was subjected to extraction with toluene. The obtained organic layer was washed with saturated saline, and then the organic layer was dried with magnesium sulfate. The mixture was filtered and the filtrate was concentrated. The obtained solution was purified by silica gel column chromatography (toluene:hexane=1:9) and then recrystallized with ethyl acetate to give 1.1 g of a target white powder at a yield of 64%. The synthesis scheme of this reference example is shown below.

### [Chemical Formula 21]

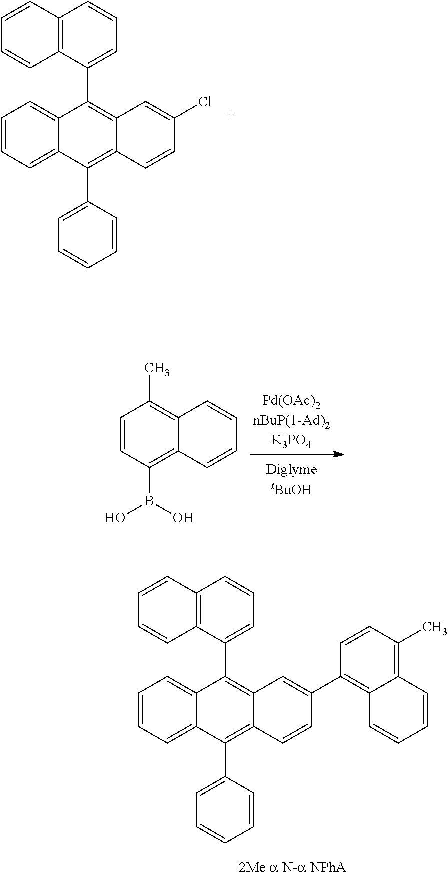

By the train sublimation method, 1.1 g of the obtained white powder was sublimated and purified. The sublimation purification was performed for 16 hours at a pressure of 3.4 Pa, an argon flow rate of 5.0 mL/min, and a heating temperature of at 240° C. After the sublimation purification, 1.0 g of a yellow powder was obtained at a collection rate of 88%.

Analysis results of the obtained yellow powder by nuclear magnetic resonance spectroscopy (¹H-NMR) are shown below. These results revealed that 2MeαN-αNPhA was obtained.

¹H NMR (CD₂Cl₂,300 MHz): δ=2.64 (s, 3H), 7.17-7.52 (m, 12H), 7.57-7.70 (m, 7H), 8.85 (d, J=8.7 Hz, 2H), 7.84 (dd, J=7.8 Hz, 1.5 Hz, 1H), 7.96-8.01 (m, 3H).

### Reference Example 2

In this reference example, a synthesis method of 9-(4-methyl-1-naphthyl)-2-(1-naphthyl)-10-phenylanthracene (abbreviation: 2αN-MeαNPhA), which is the organic compound used as a comparative example in Example, will be described in detail. The structural formula of 2αN-MeαNPhA is shown below.

### [Chemical Formula 22]

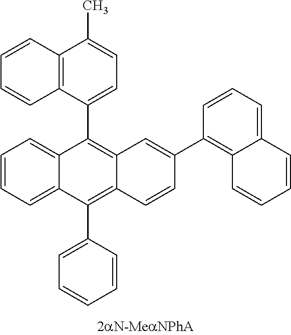 [SMILES: Cc1ccc(-c2c3ccccc3c(-c3ccccc3)c3ccc(-c4cccc5ccccc45)cc23)c2ccccc12]

Into a 200 mL three-necked flask were put 2.4 g (5.6 mmol) of 2-chloro-9-(4-methyl-1-naphthyl)-10-phenylanthracene, 1.7 g (10 mmol) of 1-naphthalene boronic acid, 0.20 g (0.56 mmol) of di(1-adamanthyl)-n-butylphosphine, 3.6 g (17 mmol) of tripotassium phosphate, and 1.2 g (17 mmol) of tert-butyl alcohol, and the air in the flask was replaced with nitrogen. To the mixture was added 28 mL of diethylene glycol dimethyl ether, and the mixture was degassed by being stirred under reduced pressure. To this mixture was added 63 mg (0.28 mmol) of palladium(II) acetate, and the mixture was stirred at 130° C. under a nitrogen stream for 3 hours.

After the stirring, water was added to this mixture, the solid obtained by performing suction filtration was dissolved in toluene, and the solution was subjected to suction filtration through Celite, alumina, and Florisil. The solid obtained by concentrating the obtained filtrate was purified by high-performance liquid chromatography (HPLC) and then recrystallized with toluene to give 2.2 g of a target pale yellow solid at a yield of 74%. The synthesis scheme of this reference example is shown below.

### [Chemical Formula 23]

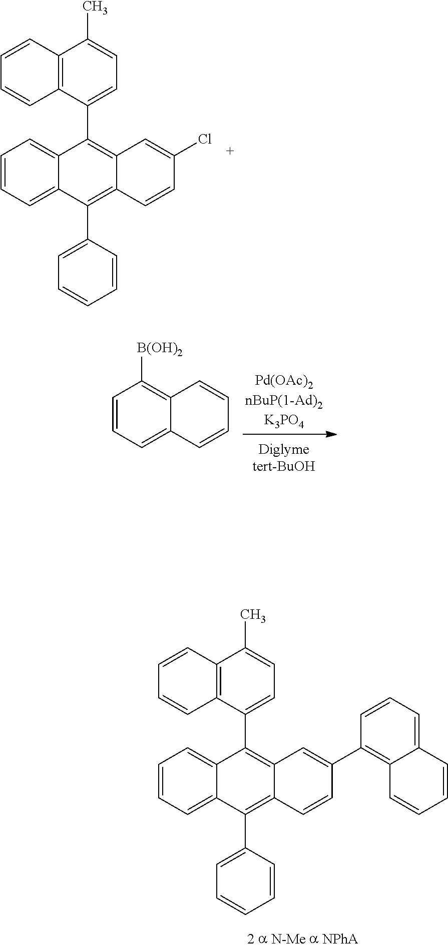

By the train sublimation method, 0.95 g of the obtained pale yellow solid was sublimated and purified. The sublimation purification was performed at a pressure of 3.6 Pa, an argon flow rate of 5.0 mL/min, and a heating temperature of 230° C. After the sublimation purification, 0.85 g of a white powder was obtained at a collection rate of 89%.

Analysis results of the obtained yellow powder by nuclear magnetic resonance spectroscopy (¹H-NMR) are shown below. These results revealed that 2αN-MeαNPhA was obtained.

¹H NMR (DMSO-d₆,300 MHz): δ=2.76 (s, 3H), 7.12 (d, J=7.5 Hz, 1H), 7.23 (t, J=6.9 Hz, 1H), 7.29-7.76 (m, 19H), 7.80 (d, J=8.7 Hz, 1H), 7.86 (d, J=8.1 Hz, 1H), 7.91 (d, J=8.1 Hz, 1H), 8.15 (d, J=8.1 Hz, 1H).

### Reference Example 3

In this reference example, a synthesis method of 2-(1-naphthyl)-10-phenyl-9-(5-phenyl-1-naphthyl)anthracene (abbreviation: 2αN-PαNPhA), which is the organic compound used as a comparative example in Example, will be described in detail. The structural formula of 2αN-PαNPhA is shown below.

### [Chemical Formula 24]

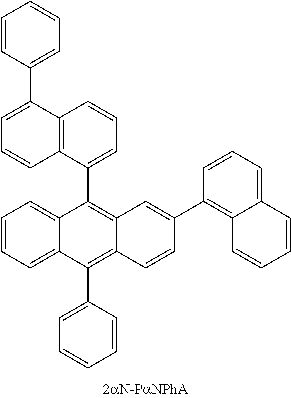 [SMILES: c1ccc(-c2cccc3c(-c4c5ccccc5c(-c5ccccc5)c5ccc(-c6cccc7ccccc67)cc45)cccc23)cc1]

Into a 50 mL three-necked flask were put 0.69 g (1.4 mmol) of 2-chloro-10-phenyl-9-(5-phenyl-1-naphthyl)anthracene, 0.48 g (2.8 mmol) of 1-naphthaleneboronic acid, 50 mg (0.14 mmol) of di(1-adamanthyl)-n-butylphosphine, 0.89 g (4.2 mmol) of tripotassium phosphate, and 0.31 g (4.2 mmol) of tert-butyl alcohol, and the air in the flask was replaced with nitrogen. To the mixture was added 7.0 mL of diethylene glycol dimethyl ether, and the mixture was degassed by being stirred under reduced pressure. To this mixture was added 16 mg (0.070 mmol) of palladium(II) acetate, and the mixture was stirred at 130° C. under a nitrogen stream for 4 hours.

After the stirring, water was added to this mixture, the solid obtained by performing suction filtration was dissolved in toluene, and the solution was subjected to suction filtration through Celite, alumina, and Florisil. The solid obtained by concentrating the obtained filtrate was purified by high-performance liquid chromatography (HPLC) and then recrystallized with toluene to give 0.65 g of a target pale yellow solid at a yield of 79%. The synthesis scheme of this reference example is shown below.

### [Chemical Formula 25]

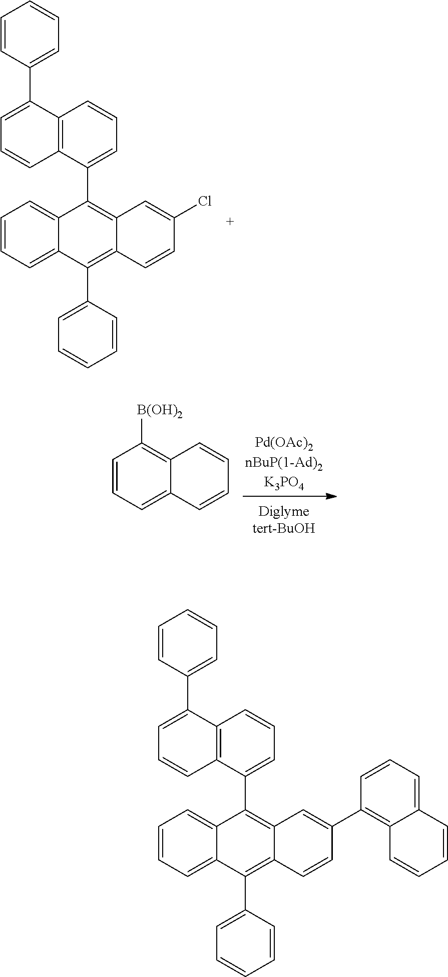

By the train sublimation method, 0.65 g of the obtained pale yellow solid was sublimated and purified. The sublimation purification was performed at a pressure of 3.6 Pa, an argon flow rate of 5.0 mL/min, and a heating temperature of 250° C. After the sublimation purification, 0.56 g of a pale yellow solid was obtained at a collection rate of 86%.

Analysis results of the obtained pale yellow solid by nuclear magnetic resonance spectroscopy (¹H-NMR) are shown below. These results revealed that 2αN-PαNPhA was obtained.

¹H NMR (DMSO-d₆, 300 MHz): δ=7.10 (d, J=7.5 Hz, 1H), 7.25 (t, J=7.5 Hz, 1H), 7.34-7.97 (m, 28H).

### Reference Example 4

In this reference example, a synthesis method of 9-(1-naphthyl)-10-phenyl-2-(5-trimethylsilyl-1-naphthyl)anthracene (abbreviation: 2TMSαN-αNPhA), which is the organic compound used as a comparative example in Example, will be described in detail. The structural formula of 2TMSαN-αNPhA is shown below.

### [Chemical Formula 26]

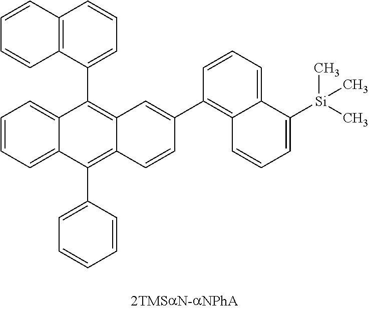 [SMILES: C[Si](C)(C)c1cccc2c(-c3ccc4c(-c5ccccc5)c5ccccc5c(-c5cccc6ccccc56)c4c3)cccc12]

Into a 200 mL three-necked flask were put 1.2 g (3.0 mmol) of 2-chloro-9-(1-naphthyl)-10-phenylanthracene, 1.2 g (3.6 mmol) of 2-(5-trimethylsilyl-1-naphthyl)-4,4,5,5-tetramethyl-1,3,2-dioxaborolan, 0.11 g (0.30 mmol) of di(1-adamanthyl)-n-butylphosphine, 1.9 g (9.1 mmol) of tripotassium phosphate, 0.71 g (9.5 mmol) of tert-butyl alcohol, and 15 mL of diethylene glycol dimethyl ether, and the mixture was degassed by being stirred under reduced pressure. To this mixture was added 38 mg (0.18 mmol) of palladium(II) acetate, and the mixture was stirred at 130° C. under a nitrogen stream for 4 hours. After the stirring, water was added to the obtained mixture, and an aqueous layer was subjected to extraction with toluene. The obtained organic layer was washed with saturated saline, and then the organic layer was dried with magnesium sulfate. The mixture was filtered and the filtrate was concentrated. The obtained solution was purified by silica gel column chromatography (toluene:hexane=1:4) to give an oily substance. The obtained oily substance was purified by high performance liquid chromatography (IPLC) to give an oily substance. Methanol was added to the obtained oily substance and the precipitated solid was collected to give 1.1 g of a target white powder at a yield of 62%. The synthesis scheme of this reference example is shown below.

### [Chemical Formula 27]

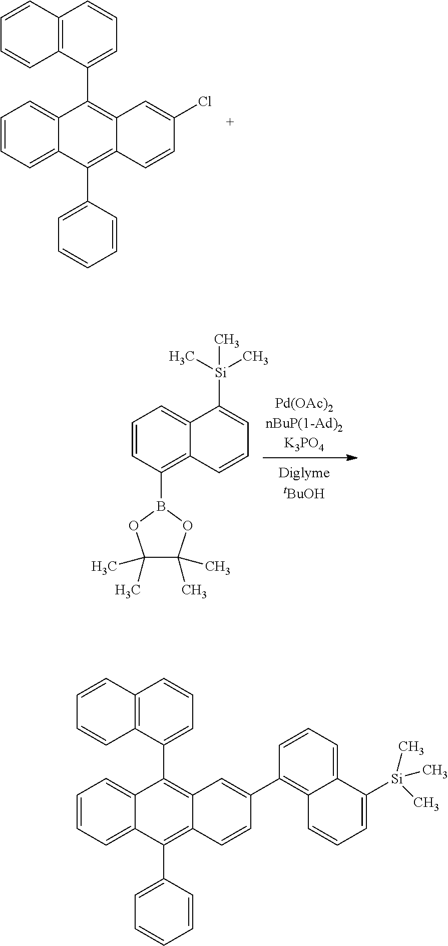 [SMILES: Clc1ccc2c(-c3ccccc3)c3ccccc3c(-c3cccc4ccccc34)c2c1 ; CC1(C)OB(c2cccc3c([Si](C)(C)C)cccc23)OC1(C)C ; C[Si](C)(C)c1cccc2c(-c3ccc4c(-c5ccccc5)c5ccccc5c(-c5cccc6ccccc56)c4c3)cccc12]

By the train sublimation method, 0.73 g of the obtained white powder was sublimated and purified. The sublimation purification was performed for 18 hours at a pressure of 3.5 Pa, an argon flow rate of 5.0 mL/min, and a heating temperature of 230° C. After the sublimation purification, 0.60 g of a pale yellow powder was obtained at a collection rate of 82%.

Analysis results of the obtained yellow powder by nuclear magnetic resonance spectroscopy (¹H-NMR) are shown below. These results revealed that 2TMSαN-αNPhA was obtained.

¹H NMR (CD₂Cl₂, 300 MHz): δ=0.42 (s, 9H), 7.14-7.52 (m, 11H), 7.57-7.72 (m, 8H), 7.78 (d, J=8.7 Hz, 2H), 7.85 (dd, J=8.7 Hz, 1.2 Hz, 1H), 7.95-8.03 (m, 3H).

### Reference Example 5

In this reference example, a synthesis method of 2-(1-naphthyl)-10-phenyl-9-(5-trimethylsilyl-1-naphthyl)anthracene (abbreviation: 2αN-TMSαNPhA), which is the organic compound used as a comparative example in Example, will be described in detail. The structural formula of 2αN-TMSαNPhA is shown below.

### [Chemical Formula 28]

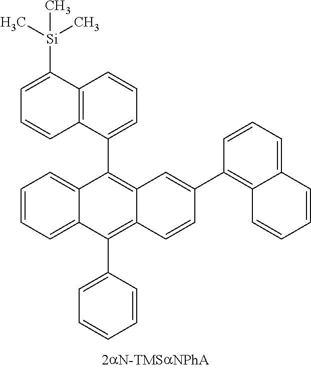 [SMILES: C[Si](C)(C)c1cccc2c(-c3c4ccccc4c(-c4ccccc4)c4ccc(-c5cccc6ccccc56)cc34)cccc12]

Into a 300 mL recovery flask were put 1.2 g (2.5 mmol) of 2-chloro-9-(1-naphthyl)-10-phenylanthracene, 0.86 g (5.0 mmol) of naphthalene-1-boronic acid, 90 mg (0.25 mmol) of di(1-adamanthyl)-n-butylphosphine, 1.6 g (7.5 mmol) of tripotassium phosphate, and 0.56 g (7.5 mmol) of tert-butyl alcohol, and the air in the flask was replaced with nitrogen. To the mixture was added 12 mL of diethylene glycol dimethyl ether, and the mixture was degassed by being stirred under reduced pressure. To this mixture was added 28 mg (0.13 mmol) of palladium(II) acetate, and the mixture was stirred at 130° C. under a nitrogen stream for 6 hours.

After the stirring, water was added to the mixture, an aqueous layer of the mixture was subjected to extraction with toluene, and the solution of the extract and the organic layer were washed with saturated saline. The organic layer was dried with magnesium sulfate, and the mixture was subjected to gravity filtration. The solid obtained by concentration of the resulting filtrate was purified by silica gel column chromatography (developing solvent, hexane:toluene=5:1) to give a solid. The obtained solid was purified by high-performance liquid chromatography (HPLC) and then recrystallized with hexane/toluene to give 1.2 g of a target pale yellow solid at a yield of 81%. The synthesis scheme of this reference example is shown below.

### [Chemical Formula 29]

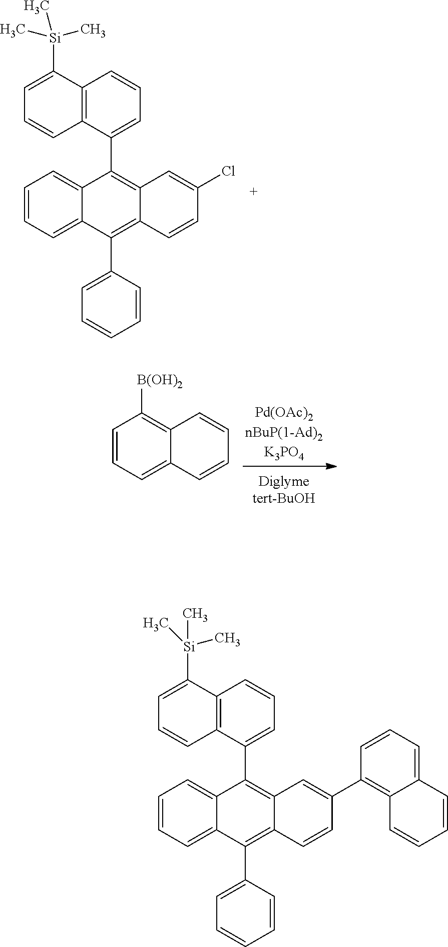 [SMILES: C[Si](C)(C)c1cccc2c(-c3c4ccccc4c(-c4ccccc4)c4ccc(Cl)cc34)cccc12 ; OB(O)c1cccc2ccccc12 ; C[Si](C)(C)c1cccc2c(-c3c4ccccc4c(-c4ccccc4)c4ccc(-c5cccc6ccccc56)cc34)cccc12]

By the train sublimation method, 1.2 g of the obtained pale yellow solid was sublimated and purified. The sublimation purification was performed at a pressure of 3.6 Pa, an argon flow rate of 5.0 mL/min, and a heating temperature of 240° C. After the sublimation purification, 1.1 g of a white solid was obtained at a collection rate of 93%.

Analysis results of the obtained pale yellow solid by nuclear magnetic resonance spectroscopy (¹H-NMR) are shown below. These results revealed that 2αN-TMSαNPhA was obtained.

¹H NMR (DMSO-d₆,300 MHz): δ=0.48 (s, 9H), 7.12-7.19 (m, 2H), 7.26-7.46 (m, 8H), 7.53-7.87 (m, 14H), 8.23 (d, J=8.1 Hz, 1H).

### Reference Example 6

In this reference example, a synthesis method of 2-(1-naphthyl)-9-(2-naphthyl)-10-phenylanthracene (abbreviation: 2αN-QNPhA), which is the organic compound used as a comparative example in Example, will be described in detail. The structural formula of 2αN-βNPhA is shown below.

### [Chemical Formula 30]

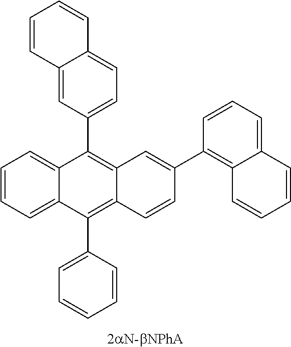 [SMILES: c1ccc(-c2c3ccccc3c(-c3ccc4ccccc4c3)c3cc(-c4cccc5ccccc45)ccc23)cc1]

Into a 200 mL recovery flask were put 2.1 g (5.0 mmol) of 2-chloro-9-(2-naphthyl)-10-phenylanthracene, 1.3 g (7.3 mmol) of 1-naphthylboronic acid, 0.36 g (1.0 mmol) of di(1-adamanthyl)-n-butylphosphine, 3.2 g (15 mmol) of tripotassium phosphate, and 1.1 g (15 mmol) of tert-butyl alcohol, and the air in the flask was replaced with nitrogen. To the mixture was added 25 mL of diethylene glycol dimethyl ether, and the mixture was degassed by being stirred under reduced pressure. To this mixture was added 0.11 g (0.50 mmol) of palladium(II) acetate, and the mixture was stirred at 130° C. under a nitrogen stream for 10 hours.

After the stirring, toluene was added to the mixture, the mixture was subjected to suction filtration, and the resulting filtrate was concentrated. The obtained solution was purified by silica gel column chromatography (developing solvent, hexane:toluene=4:1) to give an oily substance. The obtained oily substance was purified by high-performance liquid chromatography (HPLC) and then recrystallized with a mixed solvent of ethyl acetate and hexane to give 1.0 g of a target pale yellow solid at a yield of 40%.

### [Chemical Formula 31]

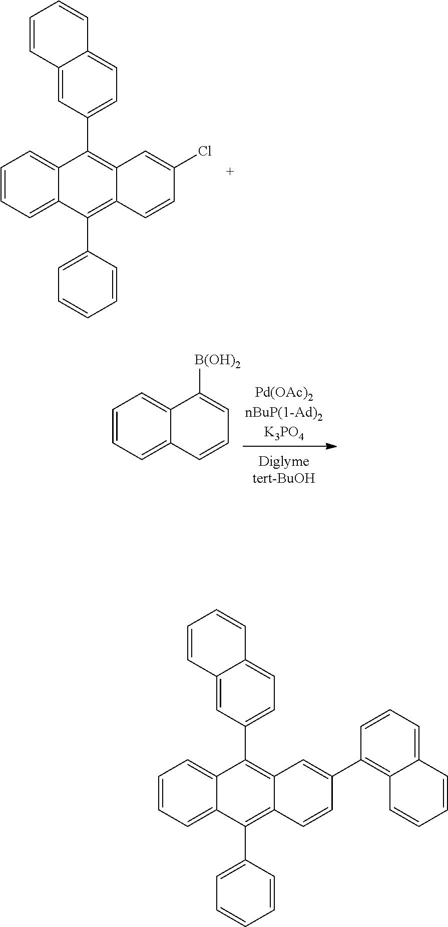

By the train sublimation method, 1.0 g of the obtained pale yellow solid was sublimated and purified. The sublimation purification was performed by heating the pale yellow solid at 230° C. under the conditions where the pressure was 3.6 Pa and the argon flow rate was 5.0 mL/min. After the sublimation purification, 0.84 g of a white solid was obtained at a collection rate of 84 5%.

Analysis results of the obtained white solid by nuclear magnetic resonance spectroscopy (¹H-NMR) are shown below. These results revealed that 2αN-βNPhA was obtained.

¹H NMR (DMSO-d₆,300 MHz): δ=7.39-7.78 (m, 19H), 7.86-7.96 (m, 3H), 8.00-8.05 (m, 2H), 8.12-8.15 (m, 2H).

### Reference Example 7

In this reference example, a synthesis method of 9-(1-naphthyl)-2-(2-naphthyl)-10-phenylanthracene (abbreviation: 2βN-αNPhA), which is the organic compound used as a comparative example in Example, will be described in detail. The structural formula of 2βN-αNPhA is shown below.

### [Chemical Formula 32]

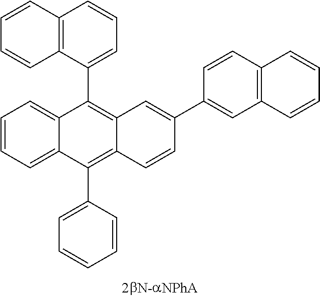 [SMILES: c1ccc(-c2c3ccccc3c(-c3cccc4ccccc34)c3cc(-c4ccc5ccccc5c4)ccc23)cc1]

Into a 200 mL recovery flask were put 1.4 g (3.3 mmol) of 2-chloro-9-(1-naphthyl)-10-phenylanthracene, 1.1 g (6.6 mmol) of 2-naphthylboronic acid, 0.12 g (0.34 mmol) of di(1-adamanthyl)-n-butylphosphine, 2.1 g (10 mmol) of tripotassium phosphate, and 0.74 g (10 mmol) of tert-butyl alcohol, and the air in the flask was replaced with nitrogen. To the mixture was added 17 mL of diethylene glycol dimethyl ether, and the mixture was degassed by being stirred under reduced pressure. To this mixture was added 37 mg (0.17 mmol) of palladium(II) acetate, and the mixture was stirred at 130° C. under a nitrogen stream for 5 hours.

After the stirring, toluene was added to the mixture, the mixture was subjected to suction filtration, and the resulting filtrate was concentrated. The obtained solution was purified by silica gel column chromatography (developing solvent, hexane:toluene=2:1) and then recrystallized with ethyl acetate/hexane to give 1.3 g of a target pale yellow solid at a yield of 77%.

### [Chemical Formula 33]

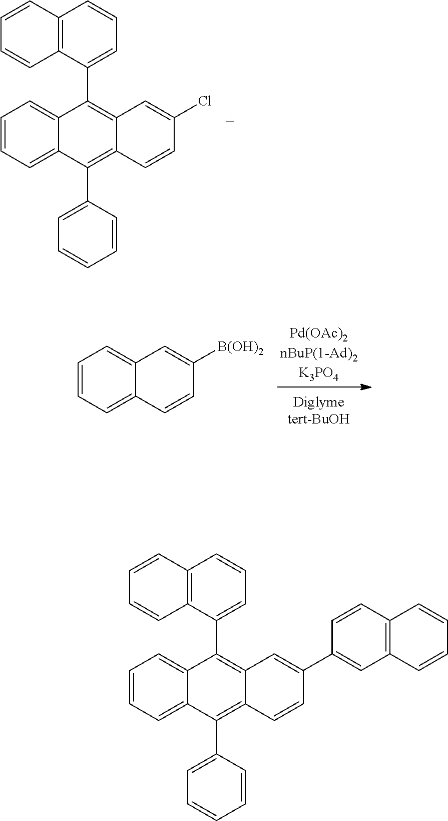

By the train sublimation method, 1.3 g of the obtained pale yellow solid was sublimated and purified. The sublimation purification was performed by heating the pale yellow solid at 210° C. under the conditions where the pressure was 3.6 Pa and the argon flow rate was 5.0 mL/min. After the sublimation purification, 1.2 g of a pale yellow solid was obtained at a collection rate of 93%.

Analysis results of the obtained pale yellow solid by nuclear magnetic resonance spectroscopy (¹H-NMR) are shown below. These results revealed that 2ρ3N-αNPhA was obtained.

¹H NMR (DMSO-d₆, 300 MHz): δ 7.04 (d, J=8.4 Hz, 1H), 7.29-7.94 (m, 22H), 7.97 (s, 1H), 8.15 (d, J=8.1 Hz, 1H), 8.23 (d, J=8.1 Hz, 1H).

### REFERENCE NUMERALS

101: first electrode, 102: second electrode, 103: EL layer, 111: hole-injection layer, 112: hole-transport layer, 112-1: first hole-transport layer, 112-2: second hole-transport layer, 113: light-emitting layer, 114: electron-transport layer, 115: electron-injection layer, 116: charge-generation layer, 117: p-type layer, 118: electron-relay layer, 119: electron-injection buffer layer, 400: substrate, 401: first electrode, 403: EL layer, 404: second electrode, 405: sealant, 406: sealant, 407: sealing substrate, 412: pad, 420: IC chip, 501: first electrode, 502: second electrode, 511: first light-emitting unit, 512: second light-emitting unit, 513: charge-generation layer, 601: driver circuit portion (source line driver circuit), 602: pixel portion, 603: driver circuit portion (gate line driver circuit), 604: sealing substrate, 605: sealant, 607: space, 608: wiring, 609: FPC (flexible printed circuit), 610: element substrate, 611: switching FET, 612: current control FET, 613: first electrode, 614: insulator, 616: EL layer, 617: second electrode, 618: light-emitting device, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: first interlayer insulating film, 1021: second interlayer insulating film, 1022: electrode, 1024W: anode, 1024R: anode, 1024G: anode, 1024B: anode, 1025: partition, 1028: EL layer, 1029: second electrode, 1031: sealing substrate, 1032: sealant, 1033: transparent base material, 1034R: red coloring layer, 1034G: green coloring layer, 1034B: blue coloring layer, 1035: black matrix, 1037: third interlayer insulating film, 1040: pixel portion, 1041: driver circuit portion, 1042: peripheral portion, 2001: housing, 2002: light source, 2100: robot, 2110: arithmetic device, 2101: illuminance sensor, 2102: microphone, 2103: upper camera, 2104: speaker, 2105: display, 2106: lower camera, 2107: obstacle sensor, 2108: moving mechanism, 3001: lighting apparatus, 5000: housing, 5001: display portion, 5002: display portion, 5003: speaker, 5004: LED lamp, 5006: connection terminal, 5007: sensor, 5008: microphone, 5012: support, 5013: earphone, 5100: cleaning robot, 5101: display, 5102: camera, 5103: brush, 5104: operation button, 5150: portable information terminal, 5151: housing, 5152: display region, 5153: bend portion, 5120: dust, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: second display portion, 7401: housing, 7402: display portion, 7403: operation button, 7404: external connection port, 7405: speaker, 7406: microphone, 9310: portable information terminal, 9311: display panel, 9313: hinge, 9315: housing

This application is based on Japanese Patent Application Serial No. 2019-024416 filed with Japan Patent Office on Feb. 14, 2019, the entire contents of which are hereby incorporated herein by reference.

1. (canceled)

2. A light-emitting device comprising:
a first electrode;
a second electrode; and
a light-emitting layer between the first electrode and the second electrode,
wherein the light-emitting layer comprises a light-emitting material and a first material represented by General Formula (G1), 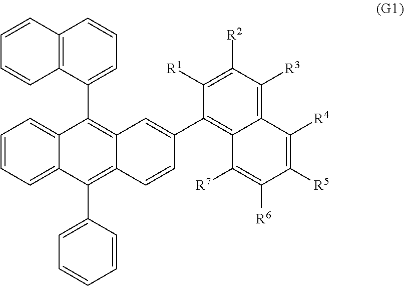
wherein R¹ to R⁷ each independently represent any one of hydrogen, a phenyl group, a naphthyl group, a fluorenyl group, a 9,9′-spirobifluorenyl group, a 9,9-diphenylfluorenyl group, an anthryl group, a phenanthryl group, a pyrenyl group, a triphenylenyl group, a fluoranthenyl group, a biphenyl group, a terphenyl group, and a quaterphenyl group.

3. A light-emitting device comprising:
a first electrode;
a second electrode; and
a light-emitting layer between the first electrode and the second electrode,
wherein the light-emitting layer comprises a light-emitting material and a first material represented by General Formula (G2), 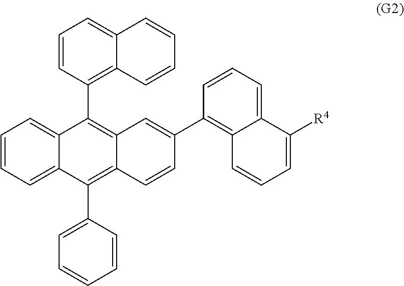
wherein R⁴ represents any one of hydrogen, a phenyl group, a naphthyl group, a fluorenyl group, a 9,9′-spirobifluorenyl group, a 9,9-diphenylfluorenyl group, an anthryl group, a phenanthryl group, a pyrenyl group, a triphenylenyl group, a fluoranthenyl group, a biphenyl group, a terphenyl group, and a quaterphenyl group.

4. The light-emitting device according to claim 2, wherein R¹ to R⁷ each represent hydrogen.

5. The light-emitting device according to claim 3, wherein R⁴ represents the phenyl group.

6. The light-emitting device according to claim 2, wherein the first material is represented by Structural Formula (100)

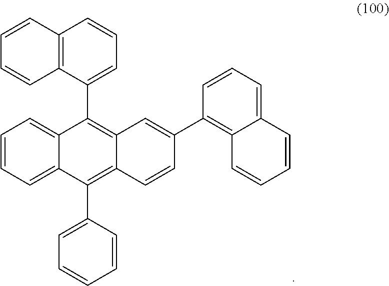 [SMILES: c1ccc(-c2c3ccccc3c(-c3cccc4ccccc34)c3cc(-c4cccc5ccccc45)ccc23)cc1]

7. The light-emitting device according to claim 3, wherein the first material is represented by Structural Formula (101)

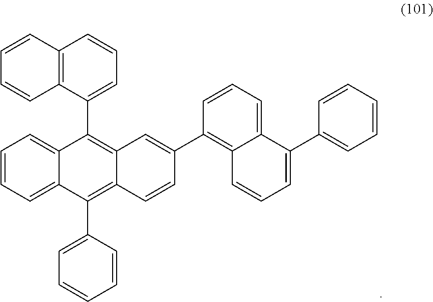 [SMILES: c1ccc(-c2cccc3c(-c4ccc5c(-c6ccccc6)c6ccccc6c(-c6cccc7ccccc67)c5c4)cccc23)cc1]

8. The light-emitting device according to claim 2, wherein the light-emitting material emits blue fluorescence.

9. The light-emitting device according to claim 3, wherein the light-emitting material emits blue fluorescence.

10. The light-emitting device according to claim 2, wherein the light-emitting material is a fluorescent substance.

11. The light-emitting device according to claim 3, wherein the light-emitting material is a fluorescent substance.

12. The light-emitting device according to claim 2, wherein the light-emitting material is a substance exhibiting thermally activated delayed fluorescence.

13. The light-emitting device according to claim 3, wherein the light-emitting material is a substance exhibiting thermally activated delayed fluorescence.

14. The light-emitting device according to claim 2,
wherein the light-emitting layer further comprises a second material, and
wherein the first material and the second material form an exciplex.

15. The light-emitting device according to claim 3,
wherein the light-emitting layer further comprises a second material, and
wherein the first material and the second material form an exciplex.

16. A light-emitting device comprising:
a first electrode;
a first light-emitting unit;
a charge-generation layer;
a second light-emitting unit; and
a second electrode,
wherein the first light-emitting unit is between the first electrode and the charge-generation layer,
wherein the charge-generation layer is between the first light-emitting unit and the second light-emitting unit,
wherein the second light-emitting unit is between the charge-generation layer and the second electrode,
wherein the first light-emitting unit comprises a first light-emitting layer,
wherein the second light-emitting unit comprises a second light-emitting layer,
wherein at least one of the first light-emitting layer and the second light-emitting layer comprises a first material represented by General Formula (G1), and 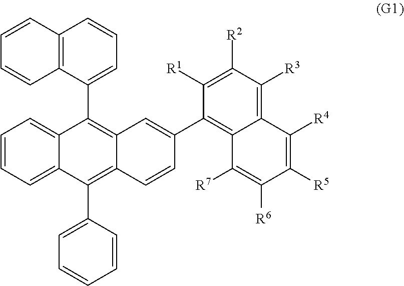
wherein R¹ to R⁷ each independently represent any one of hydrogen, a phenyl group, a naphthyl group, a fluorenyl group, a 9,9′-spirobifluorenyl group, a 9,9-diphenylfluorenyl group, an anthryl group, a phenanthryl group, a pyrenyl group, a triphenylenyl group, a fluoranthenyl group, a biphenyl group, a terphenyl group, and a quaterphenyl group.

17. The light-emitting device according to claim 16,
wherein the first material is included in the first light-emitting layer or the second light-emitting layer with a light-emitting material, and
wherein the light-emitting material emits blue fluorescence.

18. The light-emitting device according to claim 16,
wherein the first material is included in the first light-emitting layer or the second light-emitting layer with a light-emitting material, and
wherein the light-emitting material is a fluorescent substance.

19. The light-emitting device according to claim 16, wherein R¹ to R⁷ each represent hydrogen.

20. The light-emitting device according to claim 16, wherein R⁴ represents the phenyl group and R¹ to R³ and R⁵ to R⁷ each represent hydrogen.
